(19) United States
(12) Patent Application Publication
(10) Pub. No.: US 2021/0087238 A1
(43) Pub. Date: Mar. 25, 2021

(54) CELL PENETRATING PEPTIDES AND RELATED COMPOSITIONS AND METHODS

(71) Applicant: Phylogica Limited, Subiaco (AU)

(72) Inventors: Katrin Hoffmann, Subiaco (AU); Paula Cunningham, Subiaco (AU); Shane Stone, Subiaco (AU); Suzy Juraja, Subiaco (AU); Tatjana Heinrich, Subiaco (AU); Nadia Milech, Subiaco (AU); Mark Anastasas, Subiaco (AU); Maria Kerfoot, Subiaco (AU); Paul Watt, Subiaco (AU)

(21) Appl. No.: 16/634,052

(22) PCT Filed: Jul. 27, 2018
(86) PCT No.: PCT/AU2018/050781

§ 371 Date: Jan. 24, 2020

(30) Foreign Application Priority Data
Jul. 28, 2017 (AU) 2017902976

(51) Int. Cl. C07K 14/47

(52) U.S. Cl. CPC C07K 14/47; C07K 2319/01

(57) ABSTRACT

Described herein are improved cell penetrating peptides and related compositions and methods for their enhanced functional delivery.

### TECHNICAL FIELD

The present disclosure generally is directed to cell penetrating peptides (CPPs) and related compositions and methods.

### BACKGROUND

Peptides are attractive diagnostic and therapeutic agents due to their high potency and target specificity. In particular, peptides are very promising as inhibitors of intracellular protein-protein interactions (e.g., p53 interactions), which have typically been quite difficult to target using small molecule therapeutics. However, one of the challenges to more widespread adoption of peptides as therapeutics is the inability of most peptides to access intracellular targets, as the cell membrane generally acts as a barrier to intracellular entry of peptides. Further, existing cell penetrating peptides (CPPs) and any associated cargo typically become entrapped in the endosomal and lysosomal compartment. Thus, in order to fully exploit the advantages of peptide therapeutics there is an ongoing need to develop compositions and methods for intracellular/cytosolic delivery of peptides and associated payloads.

### SUMMARY

The present disclosure provides cell penetrating peptides (CPPs) and related compositions. Such compositions are particularly useful for enhancing cytosolic delivery of a linked cargo, e.g, a heterologous peptide, a heterologous, protein, or a small molecule therapeutic agent linked to a CPP. Such reagents and methods can provide for additional stability of a peptide.

Accordingly, the present disclosure provides a non-naturally occurring cell-penetrating peptide (CPP) comprising an amino acid sequence corresponding to the following structure:

X¹-X²-X³-X⁴-X⁵ (Formula I), wherein:

X¹ is an optional amino acid sequence selected from the group consisting of: QE; KTQE (SEQ ID NO:1); and RTQE (SEQ ID NO:2);

X² is any combination of 3 to 8 lysine and/or arginine residues;

X³ is an amino acid sequence selected from the group consisting of: QPAKPRPKTQE (SEQ ID NO:3), QPPKPKKPKTQE (SEQ ID NO:4), QPPRPRRPRTQE (SEQ ID NO:5), QTTKTKKTKTQE (SEQ ID NO:6), QPAKKKPKTQE (SEQ ID NO:7), and QAPKQPPKPKKPKTQE (SEQ ID NO:8)

X⁴ is any combination of 3 to 8 arginine and/or lysine residues; and

X⁵ is an amino acid sequence selected from the group consisting of QPPKPKR (SEQ ID NO:9); QTTKTKR (SEQ ID NO:10); QPPKPK (SEQ ID NO:11); and QPPRPRR (SEQ ID NO:12), wherein the amino acid sequence of the non-naturally occurring CPP does not consist of the amino acid corresponding to:

(SEQ ID NO: 88)
PLKPKKPKTQEKKKKQPPKPKKPKTQEKKKKQPPKPKR.

The present disclosure also provides a non-naturally occurring cell-penetrating peptide (CPP) comprising an amino acid sequence corresponding to the following structure:

X¹-X²-X³-X⁴-X⁵ (Formula II), wherein:

X¹ is an optional amino acid sequence selected from the group consisting of: P; QE; KTQE (SEQ ID NO:1); RTQE (SEQ ID NO:2); QPPKPKR (SEQ ID NO:223); and RKPKPPQ (SEQ ID NO:224);

X² is any combination of 3 to 8 lysine and/or arginine residues;

X³ is an amino acid sequence selected from the group consisting of SEQ ID NOs:3-8 and 225-248;

X⁴ is any combination of 3 to 8 arginine and/or lysine residues; and

X⁵ is an optional amino acid sequence selected from the group consisting of SEQ ID NOS:9-12, 249-260, and PKR, wherein the amino acid sequence of the non-naturally occurring CPP does not consist of the amino acid corresponding to:

(SEQ ID NO: 88)
PLKPKKPKTQEKKKKQPPKPKKPKTQEKKKKQPPKPKR.

In some examples X² or X⁴ consists of only arginine residues. In some examples X² consists of only arginine residues. In other examples X⁴ consists of only arginine residues. In some examples X² and X⁴ consist of only arginine residues.

In some examples X² or X⁴ consists of only lysine residues. In some embodiments X² consists of only lysine residues. In some examples X² consists of only arginine residues. In other examples X⁴ consists of only lysine residues. In some examples X² and X⁴ consist of only lysine residues.

In some examples X² or X⁴ consists of both arginine and lysine residues. In some examples X² and X⁴ consist of both arginine and lysine residues.

In some examples the length of the amino acid sequence of the CPP consists of 25 to 100 residues. In other examples the length of the amino acid sequence of the CPP consists of 30 to 70 residues. In other examples the length of the amino acid sequence of the CPP consists of 40 to 60 residues. In other examples the length of the amino acid sequence of the CPP consists of 25 to 50 residues.

In some examples the amino acid sequence of the CPP consists of Formula I or Formula II.

In some examples the amino acid sequence of the CPP comprises an amino acid sequence selected from the group consisting of SEQ ID NOs: 39, 64, 67, 69, 73-81, and SEQ ID NOs:113-167. In some examples the amino acid sequence of the CPP comprises an amino acid sequence selected from the group consisting of SEQ ID NOs: 64, 74-81, and 113-167.

In some examples any of the above-mentioned CPPs comprises multiple copies of an amino acid sequence corresponding to Formula I or Formula II. In one example, the CPP comprising multiple copies of Formula I or Formula II, comprises at least two copies of the amino acid sequence corresponding to SEQ ID NO:88. In other examples the amino acid sequence of a CPP consists of Formula I or Formula II.

In some examples a CPP is a modified CPP comprising a moiety other than a canonical amino acid. In some examples, where the CPP is a modified CPP, the moiety is selected from the group consisting of a detectable label, a non-canonical amino acid, a reactive group, a fatty acid, cholesterol, a lipid, a bioactive carbohydrate, a nanoparticle, a small molecule drug, and a polynucleotide. In some examples the moiety in a modified CPP is a D-amino acid.

In some examples the moiety is a detectable label. In some examples the detectable label is selected from the group consisting of a fluorophore, a fluorogenic substrate, a luminogenic substrate, and a biotin.

In some examples, the detectable label is a fluorophore. In some examples the fluorophore is a pH-sensitive fluorescent probe. In some examples the pH-sensitive fluorescent probe is naphthofluorescein. In other examples the moiety is a fluorogenic substrate.

In some examples, where the CPP is a modified CPP, a moiety is non-covalently linked to the CPP. In other examples the moiety is covalently linked to the CPP. In some examples the moiety is covalently linked at the N-terminal of the CPP amino acid sequence. In other examples the moiety is covalently linked at the C-terminal of the CPP amino acid sequence. In other examples the moiety is covalently linked to a sidechain of the CPP amino acid sequence. In some examples, the amino acid sequence of a CPP is the retro-inverso sequence of the amino acid sequence of the amino acid sequence of any of the foregoing CPPs.

The present disclosure also provides a CPP fusion protein comprising the amino acid sequence of any of the CPPs disclosed herein and a heterologous amino acid sequence. In some examples the heterologous amino acid sequence in the CPP fusion protein comprises an amino acid sequence selected from the group consisting of a SpyTag peptide (SEQ ID NO:84), a Phylomer™ as defined herein, a reporter protein, a pro-apoptotic peptide, a targeting protein, a bioactive peptide, a dominant negative peptide, a cytotoxic protein, an enzyme, an antibody, and a SpyC peptide (SEQ ID NO:83). In some examples the heterologous amino acid sequence comprises the amino acid sequence of a dominant negative peptide. In some examples the dominant negative peptide comprises the amino acid sequence of Omomyc (SEQ ID NO:99). In some examples the heterologous amino acid sequence comprises the amino acid sequence of β-lactamase (SEQ ID NO:112). In some examples the heterologous amino acid sequence comprises the amino acid sequence of a dominant negative peptide. In some examples the dominant negative peptide peptide comprises the amino acid sequence of Omomyc (SEQ ID NO:99). In other examples the heterologous amino acid sequence comprises the amino acid sequence of a proapoptotic peptide. In some examples the amino acid sequence of the proapoptotic peptide comprises the amino acid sequence corresponding to SEQ ID NO:61 or SEQ ID NO:63. In other examples the heterologous amino acid sequence comprises the amino acid sequence of an enzyme. In some examples the enzyme is a therapeutic enzyme. In other examples the heterologous amino acid sequence comprises the amino acid sequence of a SpyTag peptide (SEQ ID NO:84).

In some examples a CPP fusion protein comprises a flexible linker linking the CPP and the heterologous amino acid sequence.

The present disclosure further provides a CPP conjugate comprising a CPP fusion protein covalently linked to a SpyCatcher fusion protein comprising the amino acid sequence of SEQ ID NO:83 and a heterologous amino acid sequence, wherein the SpyCatcher fusion protein is covalently linked to the CPP fusion protein by an isopeptide bond to the SpyTag peptide. In some examples the SpyCatcher fusion protein in the CPP conjugate comprises an amino acid sequence selected from the group consisting of a Phylomer™ as defined herein, a reporter protein, a pro-apoptotic peptide, a targeting protein, a cytotoxic protein, an enzyme, a dominant negative peptide, and an antibody. In some examples the heterologous amino acid sequence in the SpyCatcher fusion protein comprises the amino acid sequence of a pro-apoptotic peptide. In some example the amino acid sequence of the pro-apoptotic peptide comprises the amino acid sequence of any one of SEQ ID NOs:61 and 63.

In other examples the SpyCatcher fusion protein comprises the amino acid sequence of a reporter protein in the form of an enzyme. In some examples the reporter protein comprises the amino acid sequence of a β-lactamase.

The present disclosure also provides a modified cell comprising a CPP, a CPP fusion protein, or a CPP conjugate.

The present disclosure also provides any of the above-mentioned CPPs, CPP fusion proteins, CPP conjugates, and modified cells for use as a medicament or diagnostic agent.

Also provided by the present disclosure is a method for delivering a CPP, a CPP fusion protein, or a CPP conjugate to a cell by contacting the cell with any of the CPPs, CPP fusion proteins, or CPP conjugate provided herein. In some examples the contacting is performed ex vivo. In other examples the contacting is performed in vivo.

### Key to Sequence Listing

SEQ ID NO: 1 CPP partial amino acid sequence of Formula I (X1-A)
SEQ ID NO: 2 CPP partial amino acid sequence of Formula I (X1-B)
SEQ ID NO: 3 CPP partial amino acid sequence of Formula I (X3-A)
SEQ ID NO: 4 CPP partial amino acid sequence of Formula I (X3-B)
SEQ ID NO: 5 CPP partial amino acid sequence of Formula I (X3-C)
SEQ ID NO: 6 CPP partial amino acid sequence of Formula I (X3-D)
SEQ ID NO: 7 CPP partial amino acid sequence of Formula I (X3-E)
SEQ ID NO: 8 CPP partial amino acid sequence of Formula I (X3-F)
SEQ ID NO: 9 CPP partial amino acid sequence of Formula I (X5-A)
SEQ ID NO: 10 CPP partial amino acid sequence of Formula I (X4-B)
SEQ ID NO: 11 CPP partial amino acid sequence of Formula I (X5-C)
SEQ ID NO: 12 CPP partial amino acid sequence of Formula I (X5-D)
SEQ ID NO: 13 Amino acid sequence of a Sindbis virus capsid-derived CPP (SVC1)
SEQ ID NO: 14 Amino acid sequence of a Sindbis virus capsid-derived CPP (SVC2)
SEQ ID NO: 15 Amino acid sequence of a Sindbis virus capsid-derived CPP (SVC3)
SEQ ID NO: 16 Amino acid sequence of a Sindbis virus capsid-derived CPP (SVC4)
SEQ ID NO: 17 Amino acid sequence of a Sindbis virus capsid-derived CPP (SVC5)
SEQ ID NO: 18 Amino acid sequence of a Sindbis virus capsid-derived CPP (SVC6)
SEQ ID NO: 19 Amino acid sequence of a Sindbis virus capsid-derived CPP (SVC7)
SEQ ID NO: 20 Amino acid sequence of a Sindbis virus capsid-derived CPP (SVC8)
SEQ ID NO: 21 Amino acid sequence of a Sindbis virus capsid-derived CPP (SVC9)
SEQ ID NO: 22 Amino acid sequence of a Sindbis virus capsid-derived CPP (SVC10)
SEQ ID NO: 23 Amino acid sequence of a Sindbis virus capsid-derived CPP (SVC11)
SEQ ID NO: 24 Amino acid sequence of a Sindbis virus capsid-derived CPP (SVC12)
SEQ ID NO: 25 Amino acid sequence of a Sindbis virus capsid-derived CPP (SVC13)
SEQ ID NO: 26 Amino acid sequence of a Sindbis virus capsid-derived CPP (SVC14)
SEQ ID NO: 27 Amino acid sequence of a Sindbis virus capsid-derived CPP (SVC15)
SEQ ID NO: 28 Amino acid sequence of a Sindbis virus capsid-derived CPP (SVC16)
SEQ ID NO: 29 Amino acid sequence of a Sindbis virus capsid-derived CPP (SVC17)
SEQ ID NO: 30 Amino acid sequence of a Sindbis virus capsid-derived CPP (SVC18)
SEQ ID NO: 31 Amino acid sequence of a Sindbis virus capsid-derived CPP (SVC19)
SEQ ID NO: 32 Amino acid sequence of a Sindbis virus capsid-derived CPP (SVC20)
SEQ ID NO: 33 Amino acid sequence of a Sindbis virus capsid-derived CPP (SVC21)
SEQ ID NO: 34 Amino acid sequence of a Sindbis virus capsid-derived CPP (SVC22)
SEQ ID NO: 35 Amino acid sequence of a Sindbis virus capsid-derived CPP (SVC23)
SEQ ID NO: 36 Amino acid sequence of a Sindbis virus capsid-derived CPP (SVC24)
SEQ ID NO: 37 Amino acid sequence of a Sindbis virus capsid-derived CPP (SVC25)
SEQ ID NO: 38 Amino acid sequence of a Sindbis virus capsid-derived CPP (SVC26)
SEQ ID NO: 39 Amino acid sequence of a Sindbis virus capsid-derived CPP (FPP1)
SEQ ID NO: 40 Amino acid sequence of a Sindbis virus capsid-derived CPP (SVC28)
SEQ ID NO: 41 Amino acid sequence of a Sindbis virus capsid-derived CPP (SVC29)
SEQ ID NO: 42 Amino acid sequence of a CPP-Spy Tag fusion protein (CST1)
SEQ ID NO: 43 Amino acid sequence of a CPP-Spy Tag fusion protein (CST2)
SEQ ID NO: 44 Amino acid sequence of a CPP-Spy Tag fusion protein (CST3)
SEQ ID NO: 45 Amino acid sequence of a CPP-Spy Tag fusion protein (CST4)
SEQ ID NO: 46 Amino acid sequence of a CPP-Spy Tag fusion protein (CST5)
SEQ ID NO: 47 Amino acid sequence of a CPP-Spy Tag fusion protein (CST6)
SEQ ID NO: 48 Amino acid sequence of a CPP-Spy Tag fusion protein (CST7)
SEQ ID NO: 49 Amino acid sequence of a CPP-Spy Tag fusion protein (CST8)
SEQ ID NO: 50 Amino acid sequence of a CPP-Spy Tag fusion protein (CST9)
SEQ ID NO: 51 Amino acid sequence of a CPP-Spy Tag fusion protein (CST10)
SEQ ID NO: 52 Amino acid sequence of a CPP-Spy Tag fusion protein (CST11)
SEQ ID NO: 53 Amino acid sequence of a CPP-Spy Tag fusion protein (CST12)
SEQ ID NO: 54 Amino acid sequence of a CPP-Spy Tag fusion protein (CST13)
SEQ ID NO: 55 Amino acid sequence of a CPP-Spy Tag fusion protein (CST14)
SEQ ID NO: 56 Amino acid sequence of a CPP-Spy Tag fusion protein (CST15)
SEQ ID NO: 57 Amino acid sequence of a CPP-Spy Tag fusion protein (CST16)
SEQ ID NO: 58 Amino acid sequence of a CPP-Spy Tag fusion protein (CST17)
SEQ ID NO: 59 Amino acid sequence of a CPP-Spy Tag fusion protein (CST18)
SEQ ID NO: 60 Amino acid sequence of a CPP-Spy Tag fusion protein (CST19-FPP1-SAR19-SpyT)
SEQ ID NO: 61 Amino acid sequence of a proapoptotic peptide (PAP1)
SEQ ID NO: 62 Amino acid sequence of a SpyCatcher-proapoptotic peptide fusion protein (SC-PAP)
SEQ ID NO: 63 Amino acid sequence of a proapoptotic peptide (PAP2)
SEQ ID NO: 64 Amino acid sequence of a Sindbis virus capsid-derived CPP (del-FPP1)
SEQ ID NO: 65 Amino acid sequence of a Sindbis virus capsid-derived CPP (SAR1)
SEQ ID NO: 66 Amino acid sequence of a Sindbis virus capsid-derived CPP (SAR2)
SEQ ID NO: 67 Amino acid sequence of a Sindbis virus capsid-derived CPP (SAR3)
SEQ ID NO: 68 Amino acid sequence of a Sindbis virus capsid-derived CPP (SAR4)
SEQ ID NO: 69 Amino acid sequence of a Sindbis virus capsid-derived CPP (SAR5)
SEQ ID NO: 70 Amino acid sequence of a Sindbis virus capsid-derived CPP (SAR6)
SEQ ID NO: 71 Amino acid sequence of a Sindbis virus capsid-derived CPP (SAR7)
SEQ ID NO: 72 Amino acid sequence of a Sindbis virus capsid-derived CPP (SAR9)
SEQ ID NO: 73 Amino acid sequence of a Sindbis virus capsid-derived CPP (SAR12)
SEQ ID NO: 74 Amino acid sequence of a Sindbis virus capsid-derived CPP (SAR13)
SEQ ID NO: 75 Amino acid sequence of a Sindbis virus capsid-derived CPP (SAR14)
SEQ ID NO: 76 Amino acid sequence of a Sindbis virus capsid-derived CPP (SAR15)
SEQ ID NO: 77 Amino acid sequence of a Sindbis virus capsid-derived CPP (SAR16 P_T)
SEQ ID NO: 78 Amino acid sequence of a Sindbis virus capsid-derived CPP (SAR 17)
SEQ ID NO: 79 Amino acid sequence of a Sindbis virus capsid-derived CPP (FPP1-P_T)
SEQ ID NO: 80 Amino acid sequence of a Sindbis virus capsid-derived CPP (FPP1-KT)
SEQ ID NO: 81 Amino acid sequence of a Sindbis virus capsid-derived CPP (FPP1.1)
SEQ ID NO: 82 Amino acid sequence of a Trimer of Sindbis virus capsid-derived CPP (SVC30-FPP1-3X)
SEQ ID NO:83 Amino acid sequence of SpyCatcher peptide (SCP)
SEQ ID NO:84 Amino acid sequence of SpyTag peptide (STP)
SEQ ID NO:85 Amino acid sequence of a dominant-negative ATF5 peptide (DNATF5)
SEQ ID NO:86 Amino acid sequence of a dominant negative ras-p21 peptide (DNras1)
SEQ ID NO:87 Amino acid sequence of a dominant negative ras-p21 peptide (DNras2)
SEQ ID NO:88 Amino acid sequence representing CPP according to Formula I
SEQ ID NO:89 Amino acid sequence of a Sindbis virus capsid-derived CPP (FPP1-SAR19)
SEQ ID NO:90 Amino acid sequence of a SpyCatcher-β-lactamase fusion protein. (SpyC-BLA)
SEQ ID NO:91 Amino acid sequence of a SpyCatcher-β-lactamase-CPP fusion protein (SpyC-BLA-FPP1.1).
SEQ ID NO:92 Amino acid sequence of Penetratin CPP (PenCPP)
SEQ ID NO:93 Amino acid sequence of TAT CPP (TATCPP)
SEQ ID NO:94 Amino acid sequence of LL1-SpyTag fusion protein (LL1-ST)
SEQ ID NO:95 Amino acid sequence of TAT-SpyTag fusion protein (TAT-ST)
SEQ ID NO:96 Amino acid sequence of Penetratin-SpyTag fusion protein (Pen-ST)
SEQ ID NO:97 Amino acid sequence of FPP1.1-SpyTag fusion protein (FPP1.1-ST)
SEQ ID NO:98 Amino acid sequence of FPP1.1-Omomyc fusion protein (FPP1.1-Omomyc)
SEQ ID NO:99 Amino acid sequence of Omomyc protein (OmoM)
SEQ ID NO:100 Amino acid sequence of FPP1.1_SpyC fusion protein (FPP1.1-SC)
SEQ ID NO:101 Amino acid sequence of dimer FPP1.1-SpyTag fusion protein (FPP1.1x2-ST)
SEQ ID NO:102 Amino acid sequence of FPP1.1-SpyTag fusion protein (FPP1.1-ST)
SEQ ID NO:103 Amino acid sequence of FPP2-SpyTag fusion protein (FPP2-ST)
SEQ ID NO:104 Amino acid sequence of FPP3-SpyTag fusion protein (FPP3-ST)
SEQ ID NO:105 Amino acid sequence of ^{D}PMII peptide (DPMI-I)
SEQ ID NO:106 Amino acid sequence of FPP1_^{D}PMII peptide (FPP1_DPMI-I)
SEQ ID NO:107 Amino acid sequence of TAT_^{D}PMII peptide (DPMI-I)
SEQ ID NO:108 Amino acid sequence of FPP3_^{D}PMII peptide (FPP3_DPMI-I)
SEQ ID NO:109 Amino acid sequence of EGFR Affibody-Bouganin-SpyC fusion protein (EGFRAffBd-Boug_SpyC)
SEQ ID NO:110 Amino acid sequence of SpyC_Omomyc fusion protein (SpyC_Omomyc)
SEQ ID NO:111 Amino acid sequence of FPP1.1_Avitag_SpyTag fusion protein (FPP1.1_Avi_SpyT)
SEQ ID NO:112 Amino acid sequence of β-lactamase (BLA)
SEQ ID NO:113 Amino acid sequence of SAR20 (SAR20)
SEQ ID NO:114 Amino acid sequence of SAR21 (SAR21)
SEQ ID NO:115 Amino acid sequence of SAR22 (SAR22)
SEQ ID NO:116 Amino acid sequence of SAR23 (SAR23)
SEQ ID NO:117 Amino acid sequence of SAR24 (SAR24)
SEQ ID NO:118 Amino acid sequence of SAR25 (SAR25)
SEQ ID NO:119 Amino acid sequence of SAR26 (SAR26)
SEQ ID NO:120 Amino acid sequence of SAR27 (SAR27)
SEQ ID NO:121 Amino acid sequence of SAR28 (SAR28)
SEQ ID NO:122 Amino acid sequence of SAR29 (SAR29)
SEQ ID NO:123 Amino acid sequence of SAR30 (SAR30)
SEQ ID NO:124 Amino acid sequence of SAR31 (SAR31)
SEQ ID NO:125 Amino acid sequence of SAR32 (SAR32)
SEQ ID NO:126 Amino acid sequence of SAR33 (SAR33)
SEQ ID NO:127 Amino acid sequence of SAR34 (SAR34)
SEQ ID NO:128 Amino acid sequence of SAR35 (SAR35)
SEQ ID NO:129 Amino acid sequence of SAR36 (SAR36)
SEQ ID NO:130 Amino acid sequence of SAR37 (SAR37)
SEQ ID NO:131 Amino acid sequence of SAR38 (SAR38)
SEQ ID NO:132 Amino acid sequence of SAR39 (SAR39)
SEQ ID NO:133 Amino acid sequence of SAR40 (SAR40)
SEQ ID NO:134 Amino acid sequence of SAR41 (SAR41)
SEQ ID NO:135 Amino acid sequence of SAR42 (SAR42)
SEQ ID NO:136 Amino acid sequence of SAR43 (SAR43)
SEQ ID NO:137 Amino acid sequence of SAR44 (SAR44)
SEQ ID NO:138 Amino acid sequence of SAR45 (SAR45)
SEQ ID NO:139 Amino acid sequence of SAR46 (SAR46)
SEQ ID NO:140 Amino acid sequence of SAR47 (SAR47)
SEQ ID NO:141 Amino acid sequence of SAR48 (SAR48)
SEQ ID NO:142 Amino acid sequence of SAR49 (SAR49)
SEQ ID NO:143 Amino acid sequence of SAR50 (SAR50)
SEQ ID NO:144 Amino acid sequence of SAR51 (SAR51)
SEQ ID NO:145 Amino acid sequence of SAR52 (SAR52)
SEQ ID NO:146 Amino acid sequence of SAR53 (SAR53)
SEQ ID NO:147 Amino acid sequence of SAR54 (SAR54)
SEQ ID NO:148 Amino acid sequence of SAR55 (SAR55)
SEQ ID NO:149 Amino acid sequence of SAR56 (SAR56)
SEQ ID NO:150 Amino acid sequence of SAR57 (SAR57)
SEQ ID NO:151 Amino acid sequence of SAR58 (SAR58)
SEQ ID NO:152 Amino acid sequence of SAR59 (SAR59)
SEQ ID NO:153 Amino acid sequence of SAR60 (SAR60)
SEQ ID NO:154 Amino acid sequence of SAR61 (SAR61)
SEQ ID NO:155 Amino acid sequence of SAR62 (SAR62)
SEQ ID NO:156 Amino acid sequence of SAR63 (SAR63)
SEQ ID NO:157 Amino acid sequence of SAR64 (SAR64)
SEQ ID NO:158 Amino acid sequence of SAR65 (SAR65)
SEQ ID NO:159 Amino acid sequence of SAR66 (SAR66)
SEQ ID NO:160 Amino acid sequence of SAR67 (SAR67)
SEQ ID NO:161 Amino acid sequence of SAR68 (SAR68)
SEQ ID NO:162 Amino acid sequence of SAR69 (SAR69)
SEQ ID NO:163 Amino acid sequence of SAR70 (SAR70)
SEQ ID NO:164 Amino acid sequence of SAR71 (SAR71)
SEQ ID NO:165 Amino acid sequence of SAR72 (SAR72)
SEQ ID NO:166 Amino acid sequence of SAR73 (SAR73)
SEQ ID NO:167 Amino acid sequence of SAR74 (SAR74)
SEQ ID NO:168 Amino acid sequence of SAR20-SpyTag fusion protein (SAR20-ST)
SEQ ID NO:169 Amino acid sequence of SAR21-SpyTag fusion protein (SAR21-ST)
SEQ ID NO:170 Amino acid sequence of SAR22-SpyTag fusion protein (SAR22-ST)
SEQ ID NO:171 Amino acid sequence of SAR23-SpyTag fusion protein (SAR23-ST)
SEQ ID NO:172 Amino acid sequence of SAR24-SpyTag fusion protein (SAR24-ST)
SEQ ID NO:173 Amino acid sequence of SAR25-SpyTag fusion protein (SAR25-ST)
SEQ ID NO:174 Amino acid sequence of SAR26-SpyTag fusion protein (SAR26-ST)
SEQ ID NO:175 Amino acid sequence of SAR27-SpyTag fusion protein (SAR27-ST)
SEQ ID NO:176 Amino acid sequence of SAR28-SpyTag fusion protein (SAR28-ST)
SEQ ID NO:177 Amino acid sequence of SAR29-SpyTag fusion protein (SAR29-ST)
SEQ ID NO:178 Amino acid sequence of SAR30-SpyTag fusion protein (SAR30-ST)
SEQ ID NO:179 Amino acid sequence of SAR31-Spy Tag fusion protein (SAR31-ST)
SEQ ID NO:180 Amino acid sequence of SAR32-SpyTag fusion protein (SAR32-ST)
SEQ ID NO:181 Amino acid sequence of SAR33-SpyTag fusion protein (SAR33-ST)
SEQ ID NO:182 Amino acid sequence of SAR34-SpyTag fusion protein (SAR34-ST)
SEQ ID NO:183 Amino acid sequence of SAR35-SpyTag fusion protein (SAR35-ST)
SEQ ID NO:184 Amino acid sequence of SAR36-SpyTag fusion protein (SAR36-ST)
SEQ ID NO:185 Amino acid sequence of SAR37-SpyTag fusion protein (SAR37-ST)
SEQ ID NO:186 Amino acid sequence of SAR38-SpyTag fusion protein (SAR38-ST)
SEQ ID NO:187 Amino acid sequence of SAR39-SpyTag fusion protein (SAR39-ST)
SEQ ID NO:188 Amino acid sequence of SAR40-SpyTag fusion protein (SAR40-ST)
SEQ ID NO:189 Amino acid sequence of SAR41-SpyTag fusion protein (SAR41-ST)
SEQ ID NO:190 Amino acid sequence of SAR42-SpyTag fusion protein (SAR42-ST)
SEQ ID NO:191 Amino acid sequence of SAR43-SpyTag fusion protein (SAR43-ST)
SEQ ID NO:192 Amino acid sequence of SAR44-SpyTag fusion protein (SAR44-ST)
SEQ ID NO:193 Amino acid sequence of SAR45-SpyTag fusion protein (SAR45-ST)
SEQ ID NO:194 Amino acid sequence of SAR46-SpyTag fusion protein (SAR46-ST)
SEQ ID NO:195 Amino acid sequence of SAR47-SpyTag fusion protein (SAR47-ST)
SEQ ID NO:196 Amino acid sequence of SAR48-SpyTag fusion protein (SAR48-ST)
SEQ ID NO:197 Amino acid sequence of SAR49-SpyTag fusion protein (SAR49-ST)
SEQ ID NO:198 Amino acid sequence of SAR50-SpyTag fusion protein (SAR50-ST)
SEQ ID NO:199 Amino acid sequence of SAR51-Spy Tag fusion protein (SAR51-ST)
SEQ ID NO:200 Amino acid sequence of SAR52-SpyTag fusion protein (SAR52-ST)
SEQ ID NO:201 Amino acid sequence of SAR53-SpyTag fusion protein (SAR53-ST)
SEQ ID NO:202 Amino acid sequence of SAR54-SpyTag fusion protein (SAR54-ST)
SEQ ID NO:203 Amino acid sequence of SAR55-SpyTag fusion protein (SAR55-ST)
SEQ ID NO:204 Amino acid sequence of SAR56-SpyTag fusion protein (SAR56-ST)
SEQ ID NO:205 Amino acid sequence of SAR57-SpyTag fusion protein (SAR57-ST)
SEQ ID NO:206 Amino acid sequence of SAR58-SpyTag fusion protein (SAR58-ST)
SEQ ID NO:207 Amino acid sequence of SAR59-SpyTag fusion protein (SAR59-ST)
SEQ ID NO:208 Amino acid sequence of SAR60-SpyTag fusion protein (SAR60-ST)
SEQ ID NO:209 Amino acid sequence of SAR61-SpyTag fusion protein (SAR61-ST)
SEQ ID NO:210 Amino acid sequence of SAR62-SpyTag fusion protein (SAR62-ST)
SEQ ID NO:211 Amino acid sequence of SAR63-SpyTag fusion protein (SAR63-ST)
SEQ ID NO:212 Amino acid sequence of SAR64-SpyTag fusion protein (SAR64-ST)
SEQ ID NO:213 Amino acid sequence of SAR65-SpyTag fusion protein (SAR65-ST)
SEQ ID NO:214 Amino acid sequence of SAR66-SpyTag fusion protein (SAR66-ST)
SEQ ID NO:215 Amino acid sequence of SAR67-SpyTag fusion protein (SAR67-ST)
SEQ ID NO:216 Amino acid sequence of SAR68-SpyTag fusion protein (SAR68-ST)
SEQ ID NO:217 Amino acid sequence of SAR69-SpyTag fusion protein (SAR69-ST)
SEQ ID NO:218 Amino acid sequence of SAR70-SpyTag fusion protein (SAR70-ST)
SEQ ID NO:219 Amino acid sequence of SAR71-SpyTag fusion protein (SAR71-ST)
SEQ ID NO:220 Amino acid sequence of SAR72-SpyTag fusion protein (SAR72-ST)
SEQ ID NO:221 Amino acid sequence of SAR73-SpyTag fusion protein (SAR73-ST)
SEQ ID NO:222 Amino acid sequence of SAR74-SpyTag fusion protein (SAR74-ST)
SEQ ID NO:223 CPP partial amino acid sequence of Formula I (X1-C)
SEQ ID NO:224 CPP partial amino acid sequence of Formula I (X1-D)
SEQ ID NO:225 CPP partial amino acid sequence of Formula I (X3-G)
SEQ ID NO:226 CPP partial amino acid sequence of Formula I (X3-H)
SEQ ID NO:227 CPP partial amino acid sequence of Formula I (X3-I)
SEQ ID NO:228 CPP partial amino acid sequence of Formula I (X3-J)
SEQ ID NO:229 CPP partial amino acid sequence of Formula I (X3-K)
SEQ ID NO:230 CPP partial amino acid sequence of Formula I (X3-L)
SEQ ID NO:231 CPP partial amino acid sequence of Formula I (X3-M)
SEQ ID NO:232 CPP partial amino acid sequence of Formula I (X3-N)
SEQ ID NO:233 CPP partial amino acid sequence of Formula I (X3-O)
SEQ ID NO:234 CPP partial amino acid sequence of Formula I (X3-P)
SEQ ID NO:235 CPP partial amino acid sequence of Formula I (X3-Q)
SEQ ID NO:236 CPP partial amino acid sequence of Formula I (X3-R)
SEQ ID NO:237 CPP partial amino acid sequence of Formula I (X3-S)
SEQ ID NO:238 CPP partial amino acid sequence of Formula I (X3-T)
SEQ ID NO:239 CPP partial amino acid sequence of Formula I (X3-U)
SEQ ID NO:240 CPP partial amino acid sequence of Formula I (X3-V)
SEQ ID NO:241 CPP partial amino acid sequence of Formula I (X3-W)
SEQ ID NO:242 CPP partial amino acid sequence of Formula I (X3-X)
SEQ ID NO:243 CPP partial amino acid sequence of Formula I (X3-Y)
SEQ ID NO:244 CPP partial amino acid sequence of Formula I (X3-Z)
SEQ ID NO:245 CPP partial amino acid sequence of Formula I (X3-AA)
SEQ ID NO:246 CPP partial amino acid sequence of Formula I (X3-AB)
SEQ ID NO:247 CPP partial amino acid sequence of Formula I (X3-AC)
SEQ ID NO:248 CPP partial amino acid sequence of Formula I (X3-AD)
SEQ ID NO:249 CPP partial amino acid sequence of Formula I (X5-E)
SEQ ID NO:250 CPP partial amino acid sequence of Formula I (X5-F)
SEQ ID NO:251 CPP partial amino acid sequence of Formula I (X5-G)
SEQ ID NO:252 CPP partial amino acid sequence of Formula I (X5-H)
SEQ ID NO:253 CPP partial amino acid sequence of Formula I (X5-I)
SEQ ID NO:254 CPP partial amino acid sequence of Formula I (X5-J)
SEQ ID NO:255 CPP partial amino acid sequence of Formula I (X5-K)
SEQ ID NO:256 CPP partial amino acid sequence of Formula I (X5-L)
SEQ ID NO:257 CPP partial amino acid sequence of Formula I (X5-M)
SEQ ID NO:258 CPP partial amino acid sequence of Formula I (X5-N)
SEQ ID NO:259 CPP partial amino acid sequence of Formula I (X5-P)
SEQ ID NO:260 CPP partial amino acid sequence of Formula I (X5-Q)
SEQ ID NO:261 Amino acid sequence of a flexible linker (LKR1)
SEQ ID NO:262 Amino acid sequence of a flexible linker (LKR2)
SEQ ID NO:263 Amino acid sequence of a flexible linker (LKR3)

### BRIEF DESCRIPTION OF DRAWINGS

FIG. 1 is a list of candidate Phylomer™ CPP amino acid sequences derived from Sindbis Virus capsid protein.

FIG. 2 is a schematic illustration of the SpyCatcher/CPP_SpyTag conjugates described in the Examples. The SpyCatcher/SpyTag protein ligation technology is based on the spontaneous formation of an isopeptide bond SpyTag (SpyT) and the SpyCatcher (SpyC) partner protein in an irreversible peptide-protein coupling. This technology was used to couple various CPPs (labelled as “FPP” in the figure) with different cargoes expressed as recombinant proteins. Conjugates containing the following cargoes were used: (A) Proapoptotic Peptide (PAP); (B) β-lactamase (BLA); (C); EGFR Affibody (EFGFAffiBd); (D) EGFR Affibody conjugated to Bouganin, a cytotoxic ribosomal inactivating protein (EGFRAffiBd_Boug); (E) Naphthofluorescein (pH-sensitive fluorophore moiety) (NF); (F) PASylation protein (PAS); and (G) Omomyc, dominant negative Myc peptide (Omomyc).

FIG. 3 is a table providing a summary of CPP Phylomer™ FPP1 variant testing and selection. Parameters include IC50 in viability assays where FPP1_SpyT variants deliver conjugated SpyC_proapoptotic peptide (PAP) into CHO-K1 cells, median fluorescence for flow cytometry assays measuring β-lactamase internalisation at 4 μM for FPP1_SpyT variants conjugated to SpyC_BLA protein in CHO-K1 cells, FPP peptide length, and sequence charge. FPP derivatives are aligned to the FPP1 parental sequence and the variant and/or mutation is explained (variant type). N is the number of independent PAP assays in which each FPP variant was assessed. PAP Rank refers to the relative potency of the CPPs in a cell viability assay as described in Example 1. BLA Rank refers to the relative potency of the CPPs in the fluorescent enzymatic assay described in Example 2.

FIG. 4 shows curve plots depicting percentage viability of CHO-K1 as a function of concentration of various CPP-PAP conjugates (IC50 curves). (A) Uptake of SpyC_PAP conjugated to FPP_SpyT has a dose dependent decrease in CHO-K1 cell viability for Phylomer and Penetratin-delivered protein, assessed by resazurin reduction potential. FPP1-delivered PAP conjugate showed greatest potency, followed by FPP2, Penetratin, and finally FPP3; TAT_SpyT/SpyC_PAP and SpyC_PAP treatment had no effect at all concentrations tested up to 30 μM. Calculated IC50 for each conjugate is shown below the graph. Data shown are from N independent experiments. Error bars represent standard deviation from the mean. (B) Uptake of SpyC_PAP conjugated to Phylomer FPP1_SpyT and variants shows a dose-dependent decrease in CHO-K1 cell viability, assessed by resazurin reduction potential. Delivery using FPP1-del and FPP1.1 variants showed greater potency and lower IC50 compared to the FPP1 parental sequence, while FPP1-SAR3-mediated delivery was less potent. Calculated IC50 for each conjugate is shown below the graph. Data shown are from N independent experiments. Error bars represent standard deviation from the mean. Sequences for FPP1, FPP1.1, FPP1-del, FPP1-SAR3 are shown in FIG. 3.

FIG. 5 shows plots of cell viability testing of PAP-conjugated and unconjugated CPPs: Retro-Inverso (RI) and Dimerised FPP1.1 are potent when conjugated to PAP, but are otherwise not toxic. Cell viability assay on T47D cells of FPP1.1_SpyT peptide compared to retro-inverso (RI) and dimerised variants conjugated to the SpyCatcher_PAP (SpyC_PAP) mitochondriotoxic cargo. T47D cells were seeded at 6×10³ cells per well and treated the following day with FPP-Toxic cargo conjugates. Following 48 h incubation cell viability was assessed by resazurin reduction potential. Results show that cargo delivered via FPP is toxic and that unconjugated components remain non-toxic to cells. Highest toxicity was achieved via dimerised FPP1.1 (IC50 6.3 μM) followed by the unmodified FPP1.1 and retro-inverso FPP1.1 respectively (IC50 8.7, 11.7 μM).

FIG. 6 FPPs are non-toxic to CHO-K1 cells even at high concentrations. Cell viability effect on CHO-K1 cells of Phylomer™ CPPs and variants compared to the canonical CPPs TAT and Penetratin following 24 h (A) and 48 h (B) incubation with peptides. Cell viability was assessed by resazurin reduction potential. Membrane integrity of CHO-K1 cells were assessed by LDH release following 2 h (C) and 24 h (D) incubation with peptides. In both assays Penetratin (Pen) showed intermittent, minor cytotoxicity at the highest concentrations whereas all Phylomers and the TAT peptide remained non-toxic to cells. Results are representative of 3 independent experiments. Error bars represent standard deviation from the mean of triplicate samples.

FIG. 7 shows a series of bar graphs illustrating the percentage of β-lactamase positive cells as a function of concentration of various BLA-SpyCatcher-FPP1 or FPP1.1 conjugates versus BLA-SpyCatcher fusion protein alone or a conjugate of BLA-SpyCatcher with TAT-SpyTag. The figure shows that several BLA-SpyCatcher-CPP variants exhibit intracellular delivery of BLA. Several of the FPP1 variants (e.g, FPP1.1) exhibit a higher level of BLA delivery than the canonical CPP, TAT.

FIG. 8 shows examples of FPP-mediated intracellular delivery of a range of cargoes into cells. (A) Uptake of DPMIα peptide into cells shows a dose dependent decrease in T47D cell viability when DPMIα was delivered by Phylomers FPP1 and FPP2, and to a lesser extent, by canonical FPP TAT. Penetratin-mediated delivery shows no effect (Pen). Cell viability was assessed by resazurin reduction potential after 48 h incubation with peptides. (B) Comparison of 10 μM peptide treatments shows Phylomer™ FPP-mediated and TAT delivery of DPMIα is significantly improved compared to DPMIα peptide alone. FPP1 also shows significantly improved delivery compared to TAT. Results are representative of 3 independent experiments. Error bars represent standard deviation from the mean of duplicate samples. *P<0.05, ***P<0.001, ****P<0.0001 by one-way ANOVA with Dunnett's multiple comparison test. (C) Intracellular delivery of a modified CPP comprising a phosphorodiamidate Morpholino oligomer (PMO), FPP1.1_M23D(+7-18) induces dose-dependent skipping of exon 23 of the dystrophin gene in murine H-2Kb-tsA58 myoblasts. Exon skipping can be detected by RT-PCR from doses of 50 nM FPP1.1_M23D(+7-18), but is not detected at any dose of M23D(+7-18) PMO alone or in the untreated cells (UT). (D) Tissue staining for dystrophin expression shows in vivo treatment of C57BL/10ScSnmdx mice (5 treatments over 2 weeks, at 4 nmoles per dose) of FPP1.1_M23D(+7-18) causes improved dystrophin protein levels and muscle architecture in the diaphragm, and to a lesser extent the tibialis anterior, compared to untreated C57BL/10ScSnmdx mice (Mdx untreated control) or those treated with the M23D(+7-18) morpholino oligonucleotide alone (M23D(+7-18)-PMO). Tissue staining for dystrophin expression in C57BL/10ScSn mice (C57 untreated control) shows normal muscle architecture for comparison.

FIG. 9 FPP compatibility with receptor binding delivery (RBD)-mediated delivery and half-life extension by PASylation. (A) a plot showing percent viability of CHO-K1 cells stably-expressing EGFR receptor or (B) CHO-K1 cells were treated with FPP_SpyT conjugated to EGFRAffybody_Bouganin SpyC (EGRFAffbd_Boug_SpyC) toxin. After 48 h incubation cell viability was assessed by resazurin reduction potential. Comparison of 100 nM toxin treatment in CHO-K1 EGFR (A, right) vs CHO-K1 (B, right) cells shows that conjugation to FPP1-del significantly improved delivery compared to RBD alone but retains RBD encoded specificity. Results are representative of 3 independent experiments. Error bars represent standard deviation from the mean of duplicate samples. **P<0.01, ***P<0.001 by one-way ANOVA with Dunnett's multiple comparison test. (C) T47D cells were treated with FPP_PAP_linker_SpyT with and without conjugation to PAS_SpyC recombinant protein. FPP-dependent PAP-induced cytotoxicity was still detected for all PAS conjugates compared to the buffer control (Tris), with the Furin-cleavable conjugate showing the greatest potency. Linkers are Cathepsin B FKFL cleavage motif (BF), Cathepsin B Valine-Citrulline cleavage motif (Ba) and Furin RKKR cleavage motif (Fur). Results are representative of 2 independent experiments. Error bars represent standard deviation from the mean of duplicate samples.

FIG. 10 shows a series of plots characterizing the delivery and mechanism uptake of the CPP FPP1.1. (A) Intracellular delivery of FPP1.1 is temperature dependent, as shown by comparison of FPP-driven uptake over 150 minutes at 4° C. and 37° C. in HEK-293_BirA cells treated with 5 μM FPP1.1_SpyC_V5 conjugated to Naphthofluorescein (NF)-LL1-SpyTag. The percentage of NF-positive live single cells are plotted as a function of time. The results of two independent experiments are presented, showing mean signal after subtraction of background fluorescence, with error bars representing standard deviation from the mean. (B) Pre-treatment of HEK-293_BirA cells with endocytotic inhibitors (100 μM DMA or 20 μM Dyngo4a) reduces the efficiency of FPP-driven intracellular uptake when cells are subsequently treated with 5 μM FPP1.1_SpyC protein conjugated to NF-LL1-SpyT. Data shown are from 3 (DMA, DMSO (vehicle control) or 2 (Dyngo4a) independent experiments, pooling data from separate experiments. Error bars represent the standard deviation from the mean. (C) Pre-treatment of HEK-293_BirA cells with heparinase III (3 mIU) reduces FPP-driven intracellular delivery of recombinant FPP1.1_SpyC conjugated to NF-LL1-SpyT. Intracellular delivery of SpyC/NF-LL1-SpyT conjugate (assay negative control) is not detected regardless. Data shown are from two independent experiments, pooling data from separate experiments. Error bars represent the standard deviation from the mean. (D) Pre-incubation of FPP1.1_SpyC/NF-LL1-SpyT conjugates with varying amounts of HSPG protein reduces FPP-driven intracellular uptake of the conjugate into HEK-293_BirA cells compared to peptide pre-treated with PBS alone. Data shown are from 2 independent experiments, pooling data from separate experiments. Error bars represent the standard deviation from the mean.

FIG. 11 shows an immunoblot demonstrating that Phylomer™ FPP1.1-delivered Omomyc (a Myc dominant negative peptide) can be captured and detected in the cytoplasm of cells. HEK-293_BirA cells were treated with 15 μM of FPP1.1_Avi_SpyTag/SpyC_Omomyc conjugate or pre-biotinylated FPP1.1_Avi_SpyTag/SpyC_Omomyc conjugate for 30 mins and 60 mins. Biotinylated protein was captured on Streptavidin magnetic beads, denatured, separated on a 12% Bis-Tris gel by SDS-PAGE, before immunoblotting and imaging to detect V5-tagged proteins and peptides. Quantitation by densitometry (ChemiDoc Gel Imaging System) shows 26% (30 mins) and 46% (60 mins) intracellular uptake of the FPP1.1_Avi_SpyTag/SpyC_Omomyc conjugate compared to the partially biotinylated control.

FIG. 12 shows a series of graphs indicating cell viability and demonstrating FPP1.1-mediated functional delivery of the MYC dominant negative peptide, Omomyc. Treatment of (A) AMO-1 (plasmacytoma), (B) HL-60 (promyelocytic leukemia), and (C) T47D (breast cancer) cell lines with FPP-1.1_Omomyc protein. After 48 h incubation, cell viability was assessed by measuring ATP activity. Results show strong, similar efficacy of FPP1.1_Omomyc across all three cell lines. Peptide FPP1.1 alone shows no significant cytotoxicity. Control protein Omomyc exhibits a minor effect on cell viability only at the highest concentrations (mid to high micromolar potencies). Results are representative of two independent experiments. Error bars represent standard deviation from the mean of duplicate samples.

FIG. 13 shows a series of graphs of cell viability demonstrating that FPP1.1-delivered Omomyc is more potent than known small molecule inhibitors MYC. Treatment of AMO-1 (plasmacytoma), HL-60 (promyelocytic leukemia), and T47D (breast cancer) cell lines with FPP1.1_Omomyc or small molecule inhibitors. After a 48 h incubation, the cell viability was assessed by measuring ATP activity. Results show strong, similar efficacy of FPP1.1_Omomyc across all three cell lines that is greater than the potency of MYC small molecule inhibitors 10058-F4 (Huang et al 2006, Exp Hematol, 34, 1480-1489) and KJ-Pyr9. (Hart et al 2014, Proc Natl Acad Sci USA, 111, 12556-12561.) Phylomer™ FPP1.1 alone and DMSO alone (vehicle control for small molecule inhibitors) show no significant cytotoxicity. Control protein Omomyc (alone) exhibits a minor effect on cell viability only at the highest concentrations (mid to high micromolar potencies). Results are from one independent experiment. Error bars represent standard deviation from the mean of duplicate samples.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Each example of the present disclosure described herein is to be applied mutatis mutandis to each and every other example unless specifically stated otherwise.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure, as described herein.

The present disclosure is performed without undue experimentation using, unless otherwise indicated, conventional techniques of molecular biology, microbiology, virology, recombinant DNA technology, peptide synthesis in solution, solid phase peptide synthesis, and immunology. Such techniques are described and explained throughout the literature in sources such as Perbal 1984, Sambrook et al., 2001, Brown (editor) 1991, Glover and Hames (editors) 1995 and 1996, Ausubel et al. including all updates until present, Coligan et al. (editors) (including all updates until present), Maniatis et al. 1982, Gait (editor) 1984, Hames and Higgins (editors) 1984, Freshney (editor) 1986.

The term “and/or”, e.g, “X and/or Y” shall be understood to mean either “X and Y” or “X or Y” and shall be taken to provide explicit support for both meanings or for either meaning.

The term “about”, unless stated to the contrary, refers to +/−20%, more preferably +/−10%, of the designated value. For the avoidance of doubt, the term “about” followed by a designated value is to be interpreted as also encompassing the exact designated value itself (for example, “about 10” also encompasses 10 exactly).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term antibody” as used herein includes polyclonal antibodies, monoclonal antibodies, bispecific antibodies, fusion diabodies, triabodies, heteroconjugate antibodies, and chimeric antibodies. Also contemplated are antibody fragments that retain at least substantial (about 10%) antigen binding relative to the corresponding full length antibody. Antibodies include modifications in a variety of forms including, for example, but not limited to, domain antibodies including either the VH or VL domain, a dimer of the heavy chain variable region (VHH, as described for a camelid), a dimer of the light chain variable region (VLL), Fv fragments containing only the light (VL) and heavy chain (VH) variable regions which may be joined directly or through a linker, or Fd fragments containing the heavy chain variable region and the CH1 domain.

A scFv consisting of the variable regions of the heavy and light chains linked together to form a single-chain antibody and oligomers of scFvs such as diabodies and triabodies are also encompassed by the term “antibody”. Also encompassed are fragments of antibodies such as Fab, (Fab′)2 and FabFc2 fragments which contain the variable regions and parts of the constant regions. Complementarity determining region (CDR)-grafted antibody fragments and oligomers of antibody fragments are also encompassed. The heavy and light chain components of an Fv may be derived from the same antibody or different antibodies thereby producing a chimeric Fv region. The antibody may be of animal (for example mouse, rabbit or rat) or human origin or may be chimeric or humanize.

The antibodies may be Fv regions comprising a variable light (VL) and a variable heavy (VH) chain in which the light and heavy chains may be joined directly or through a linker. As used herein a linker refers to a molecule that is covalently linked to the light and heavy chain and provides enough spacing and flexibility between the two chains such that they are able to achieve a conformation in which they are capable of specifically binding the epitope to which they are directed. Protein linkers are particularly preferred as they may be expressed as an intrinsic component of the Ig portion of the fusion polypeptide.

In some examples, an antibody is a recombinantly produced single chain scFv antibody, preferably a humanized scFv. Methods for generating antibody fusion proteins are known in the art as exemplified in, e.g., U.S. Pat. No. 8,142,781.

The term “canonical amino acid” refers to an amino acid encoded directly by the codons of the universal genetic code. The canonical amino acids are: Alanine, Arginine, Asparagine, Aspartic acid, Cysteine, Glutamic acid, Glutamine, Glycine, Histidine, Isoleucine, Leucine, Lysine, Methionine, Phenylalanine, Proline, Serine, Threonine, Tryptophan, Tyrosine, and Valine.

The term “conjugate,” as used herein, refers to two or more peptides or proteins that are covalently linked by a means other than an amide bond between the C-terminus of one protein and the N-terminus of the other. Typically, the covalent bond is by means of an isopeptide bond formed between a sidechain carboxylic acid of one protein or peptide to be conjugated.

The term “endogenous” or “endogenously encoded” in reference to a nucleotide or amino acid sequence indicates that sequence in question is native to a virus, cell, or organism that has not been experimentally modified to encode or express the amino acid sequence in question.

A “heterologous amino acid sequence” refers to an amino acid sequence that does not naturally occur as a sequence that is contiguous with the amino acid sequence of a reference sequence. For example, green fluorescent protein is a heterologous amino acid sequence with respect to a cell penetrating peptide (CPP) derived from a Sindbis viral coat.

A “nanoparticle” refers to a microscopic particle with at least one dimension less than 100 nm. Examples of nanoparticles include, but are not limited to, derivatized gold nanoparticles, quantum dots, and polymeric nanoparticles.

The term “non-naturally occurring” in reference to a peptide will be understood to indicate that: (i) there is no endogenous gene or open reading frame that encodes an amino acid sequence consisting of the amino acid sequence of the peptide in question; and (ii) there is no endogenous protein fragment the amino acid sequence of which consists of the peptide in question. For example, a peptide consisting of the amino acid sequence of a fragment of an endogenously expressed protein is considered a non-naturally occurring peptide if the protein fragment itself is not naturally expressed or does not ordinarily occur as a byproduct of the endogenously expressed protein.

The term “Phylomer™” refers to a peptide of about 8 to about 180 amino acids encoded by nucleic acid fragments obtainable from genome(s) of a microorganisms and/or a eukaryotic species having a compact genome.

The term “peptide” is intended to include compounds composed of amino acid residues linked by amide bonds. A peptide may be natural or unnatural, ribosome encoded or synthetically derived. Typically, a peptide will consist of between 2 and 200 amino acids. For example, the peptide may have a length in the range of 10 to 20 amino acids or 10 to 30 amino acids or 10 to 40 amino acids or 10 to 50 amino acids or 10 to 60 amino acids or 10 to 70 amino acids or 10 to 80 amino acids or 10 to 90 amino acids or 10 to 100 amino acids, including any length within said range(s). The peptide may comprise or consist of fewer than about 150 amino acids or fewer than about 125 amino acids or fewer than about 100 amino acids or fewer than about 90 amino acids or fewer than about 80 amino acids or fewer than about 70 amino acids or fewer than about 60 amino acids or fewer than about 50 amino acids.

Peptides, as referred to herein, include “inverso” peptides in which all L-amino acids are substituted with the corresponding D-amino acids, “retro-inverso” peptides in which the sequence of amino acids is reversed and all L-amino acids are replaced with D-amino acids.

Peptides may comprise amino acids in both L- and/or D-form. For example, both L- and D-forms may be used for different amino acids within the same peptide sequence. In some examples the amino acids within the peptide sequence are in L-form, such as natural amino acids. In some examples the amino acids within the peptide sequence are a combination of L- and D-form.

Peptides may be encoded by nucleic acid fragments of genomic DNA or cDNA obtained from an evolutionary diverse range of organisms from Viruses, Bacteria, Archaea, and Eukarya. For example, nucleic acid fragments may be obtained from Aeropyrum pernix, Aquifex aeolicus, Archaeoglobus fulgidis, Bacillus subtilis, Bordetella pertussis, Borrelia burgdorferi, Chlamydia trachomatis, Escherichia coli, Haemophilus influenzae, Helicobacter pylori, Methanobacterium thermoautotrophicum, Methanocaldococcus jannaschii, Mycoplasma pneumoniae, Neisseria meningitidis, Pseudomonas aeruginosa, Pyrococcus horikoshii, Synechocystis PCC 6803, Thermoplasma volcanium and Thermotoga maritima. Alternatively, peptides may be synthesized using well known solid phase peptide synthesis techniques, and purification techniques.

Nucleic acid fragments may be generated using one or more of a variety of methods known to those skilled in the art. Suitable methods include, as well as those described in the examples below, for example, mechanical shearing (e.g by sonication or passing the nucleic acid through a fine gauge needle), digestion with a nuclease (e.g DNAse 1), partial or complete digestion with one or more restriction enzymes, preferably frequent cutting enzymes that recognize 4-base restriction enzyme sites and treating the DNA samples with radiation (e.g gamma radiation or ultra-violet radiation).

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical bond or a disulfide bond. Examples of non-covalent bonds include hydrogen bonds, ionic bonds, Van der Waals forces, and hydrophobic interactions.

Percentage amino acid sequence identity with respect to a given amino acid sequence is defined as the percentage of amino acid residues in a candidate sequence that are identical to the amino acid residues in the reference sequence, after aligning the sequences and introducing gaps, if necessary, to achieve the maximum percent sequence identity, and not considering any conservative substitutions as part of the sequence identity. Amino acid sequence identity may be determined using the EMBOSS Pairwise Alignment Algorithms tool available from The European Bioinformatics Institute (EMBL-EBI), which is part of the European Molecular Biology Laboratory. This tool is accessible at the website located at www.ebi.ac.uk/Tools/emboss/align/. This tool utilizes the Needleman-Wunsch global alignment algorithm (Needleman and Wunsch, 1970). Default settings are utilized which include Gap Open: 10.0 and Gap Extend 0.5. The default matrix “Blosum62” is utilized for amino acid sequences and the default matrix.

The term “cell penetrating peptide” (CPP) refers to a peptide that is capable of crossing a cellular membrane. In one example, a CPP is capable of translocating across a mammalian cell membrane and entering into a cell. In another example, a CPP may direct a conjugate to a desired subcellular compartment. Thus, a CPP may direct or facilitate penetration of a molecule of interest across a phospholipid, mitochondrial, endosomal, lysosomal, vesicular, or nuclear membrane. CPPs that are able to “escape” the endosomal and lysosomal compartments for cytosolic delivery can be referred to as “FPPs” as described herein. A CPP may be translocated across the membrane with its amino acid sequence complete and intact, or alternatively partially degraded.

A CPP may direct a molecule of interest from outside a cell through the plasma membrane, and into the cytoplasm or a desired subcellular compartment. Alternatively, or in addition, a CPP may direct a molecule of interest across the blood-brain, trans-mucosal, hematoretinal, skin, gastrointestinal and/or pulmonary barriers.

Accordingly, a CPP may be linked to a molecule of interest. Such molecules include a further peptide or protein, an RNAi agent, a therapeutic agent, a toxin, or a detectable label. The linkage may be through a covalent bond or non-covalent interactions. For example, a CPP may be linked to a further peptide or protein via a “peptide linker”. Alternatively, a CPP may be linked to another moiety (including a peptide) by a non-peptide synthetic linker. The further peptide or protein may be designed to act upon a particular intracellular target or to direct its transport to particular subcellular compartment. In some examples, a “therapeutic agent” is a small molecule compound (generally less than about 900 daltons in size). In some examples a small molecule compound is a chemotherapeutic agent, a cytotoxic molecule, or a cytostatic molecule.

The capability to translocate across membranes of a CPP may be energy dependent or independent, and/or receptor dependent or independent. In some examples, the CPP is a peptide which is demonstrated to translocate across a plasma membrane as determined by the methods described herein. CPPs encompass: (i) peptides that become internalized by cells but subsequently entrapped within endosomes or lysosomes; and (ii) peptides that not only become internalized by cells, but also are able to escape endosomal and/or lysosomal compartments once internalized by cells. The latter are referred to as “functional penetrating peptides,” (FPPs) as described herein.

The term “functional penetrating peptide” (FPP) refers to a subset of CPPs that in addition being able to mediate intracellular delivery, is also able to escape from endosomal and/or lysosomal compartments for delivery into cytosol

The term “basic amino acid” relates to any amino acid, including natural and non-natural amino acids, that has an isoelectric point above 6.3, as measured according to Kice & Marvell “Modern Principles of organic Chemistry” (Macmillan, 1974) or Matthews and van Holde “Biochemistry” Cummings Publishing Company, 1996. Included within this definition are Arginine, Lysine, Homoarginine (Har), and Histidine as well as derivatives thereof. Suitable non-natural basic amino acids are described in U.S. Pat. No. 6,858,396.

Accordingly, in some examples provided herein is a non-naturally occurring cell-penetrating peptide (CPP) comprising an amino acid sequence corresponding to the following structure:

X⁴-X²-X³-X⁴-X⁵ (Formula I), wherein:

X¹ is an optional amino acid sequence selected from the group consisting of: QE; KTQE (SEQ ID NO:1); and RTQE (SEQ ID NO:2);

X² is any combination of 3 to 8 lysine and/or arginine residues;

X³ is an amino acid sequence selected from the group consisting of:

(SEQ ID NO: 3)
QPAKPRPKTQE,
(SEQ ID NO: 4)
QPPKPKKPKTQE,
(SEQ ID NO: 5)
QPPRPRRPRTQE,
(SEQ ID NO: 6)
QTTKTKKTKTQE,
(SEQ ID NO: 7)
QPAKKKPKTQE,
and
(SEQ ID NO: 8)
QAPKQPPKPKKPKTQE

X⁴ is any combination of 3 to 8 arginine and/or lysine residues; and

X⁵ is an amino acid sequence selected from the group consisting of QPPKPKR (SEQ ID NO:9); QTTKTKR (SEQ ID NO:10); QPPKPK (SEQ ID NO:11); and QPPRPRR (SEQ ID NO:12), wherein the amino acid sequence of the non-naturally occurring CPP does not consist of the amino acid corresponding to:

(SEQ ID NO: 88)
PLKPKKPKTQEKKKKQPPKPKKPKTQEKKKKQPPKPKR.

Also provided herein is provides a non-naturally occurring cell-penetrating peptide (CPP) comprising an amino acid sequence corresponding to the following structure:

X¹-X²-X³-X⁴-X⁵ (Formula II), wherein:

X¹ is an optional amino acid sequence selected from the group consisting of: P; QE; KTQE (SEQ ID NO:1); RTQE (SEQ ID NO:2); QPPKPKR (SEQ ID NO:223); and RKPKPPQ (SEQ ID NO:224);

X² is any combination of 3 to 8 lysine and/or arginine residues;

X³ is an amino acid sequence selected from the group consisting of SEQ ID NOs:3-8 and 225-248;

X⁴ is any combination of 3 to 8 arginine and/or lysine residues; and

X⁵ is an optional amino acid sequence selected from the group consisting of SEQ ID NOS:9-12, 249-260, and PKR, wherein the amino acid sequence of the non-naturally occurring CPP does not consist of the amino acid corresponding to:

(SEQ ID NO: 88)
PLKPKKPKTQEKKKKQPPKPKKPKTQEKKKKQPPKPKR.

In some examples, X² or X⁴ consists of only arginine residues. In some examples, X² and X⁴ consist of only arginine residues. In other examples, X² or X⁴ consists of only lysine residues. In some examples, X² and X⁴ consist of only arginine residues. In further examples, X² or X⁴ consists of both arginine and lysine residues. In other examples, each of X² and X⁴ consist of both arginine and lysine residues.

In some examples, a CPP will comprise between one and ten conservative amino acid substitutions relative to any sequence described herein, e.g, 1, 2, 3, 4, 5, 6, 7, 8, 9, or 10 conservative amino acid substitutions.

A “conservative” amino acid substitution is one in which an amino acid residue is replaced with another amino acid residue having a side chain with similar physicochemical properties. Amino acid residues having side chains with similar physiochemical properties are known in the art, and include amino acids with basic side chains (e.g, lysine, arginine, histidine), acidic side chains (e.g, aspartic acid, glutamic acid), uncharged polar side chains (e.g, glycine, asparagine, glutamine, serine, threonine, tyrosine, cysteine, tryptophan), nonpolar side chains (e.g, alanine, valine, leucine, isoleucine, proline, phenylalanine, methionine), beta-branched side 10 chains (e.g, threonine, valine, isoleucine) and aromatic side chains (e.g, tyrosine, phenylalanine, tryptophan, histidine). Conservative amino acid substitutions include those with amino acids, which have been substituted with non-naturally occurring amino acids and non-proteogenic amino acids, which are therefore not among the regular amino acids encoded by the genetic code. Examples of non-proteogenic amino acids include, but are not limited to, ornithine, citrulline (Cit), diaminobutyric acid (Dab), diaminopropionic acid (Dap), 2-Aminoisobutyric acid, α-Amino-n-butyric acid, Norvaline, Norleucine, Alloisoleucine, t-leucine, Ornithine, Allothreonine, β-Alanine, β-Amino-n-butyric acid, N-isopropyl glycine, Isoserine, and Sarcosine and pyroglutamic acid. Conservative amino acid substitutions further include D-amino acids. In some examples the amino acid sequence of a CPP is a retro-inverso amino acid sequence.

In some examples the amino acid sequence of any of the foregoing CPPs consists of 25 to 100 residues, e.g, 30, 35, 40, 45, 48, 50, 52, 60, 65, 70, 75, 80, 85, 90, 95, or another number of residues from 25 to 100. In other examples the amino acid sequence of any of the foregoing CPPs consists of 30 to 70 residues, e.g, 35, 40, 45, 48, 50, 52, 60, 65, or another number of residues from 30 to 70 residues. In other examples the amino acid sequence of any of the foregoing CPPs consists of 40 to 60 residues, e.g, 42, 43, 45, 48, 50, 52, 54, 57, 58, or another number of residues from 40 to 60 residues. In some examples, the amino acid sequence of any of the foregoing CPPs consists of 35 to 50 residues, e.g, 36, 38, 40, 42, 43, 45, 57, 58, or another number of residues from 35 to 50 residues. In yet other examples the amino acid sequence of any of the foregoing CPPs consists of 25 to 50 residues, e.g, 27, 28, 30, 32, 35, 37, 38, 40, 42, 46, 48, or another number of residues from 25 to 50.

In one example the amino acid sequence of the CPP consists of an amino acid sequence corresponding to Formula I. For the avoidance of doubt, it is to be understood that in such examples, while the amino acid sequence of the CPP consists of an amino acid sequence corresponding to Formula I, the CPP may, nevertheless, comprise chemical modifications that do not alter the amino acid sequence. Such modifications include, but are not limited to, non-peptide linkers, non-peptide therapeutic agents (e.g, a chemotherapeutic agent), and detectable labels. In such examples the CPP is generally referred to as a “modified CPP,” as described in further detail herein. In other examples the CPP consists of an amino acid sequence corresponding to Formula I.

In particular examples, the amino acid sequence of the CPP comprises an amino acid sequence selected from the group consisting of SEQ ID NOs: 39, 64, 67, 69, 73-81, and SEQ ID NOs:113-167. In other examples, the amino acid sequence of the CPP consists of an amino acid sequence selected from the group consisting of SEQ ID NOs: 64, 74-81, and 113-167.

In some examples any of the above-mentioned CPPs comprise multiple copies of an amino acid sequence corresponding to Formula I or Formula II, referred to herein as a multimeric CPP. In some examples, a multimeric CPP comprises between two and ten copies of an amino acid sequence corresponding to Formula I or Formula II, e.g, 2, 3, 4, 5, 6, 7, 8, 9, or 10 copies of an amino acid sequence corresponding to Formula I or Formula II. In some examples, the multimeric CPP corresponding comprises at least two amino acid sequences selected from the group consisting of 39, 64, 67, 69, 73-81, and 113-167. In one example, the CPP comprising multiple copies of Formula I or Formula II, comprises at least two copies of the amino acid sequence corresponding to SEQ ID NO:88.

In some examples a CPP is a modified CPP comprising a moiety other than a canonical amino acid. Such modified CPPs may confer additional functionalities to a CPP, such as facilitating detection of CPP entry, localisation within cells, enhanced cell entry, and/or reduced CPP degradation in vitro or in vivo. Suitable moieties for a modified CPP include, but are not limited to any moiety selected from the group consisting of: a detectable label, a non-canonical amino acid, a reactive group, a fatty acid, cholesterol, a bioactive carbohydrate, a lipid, a nanoparticle, a small molecule drug, and a polynucleotide. In some examples the moiety in a modified CPP is a D-amino acid. In some examples the moiety in a modified CPP is a detectable label.

The term “detectable label” refers to any type of molecule which can be detected by optical, fluorescent, isotopic imaging or by mass spectroscopic techniques, or by performing simple enzymatic assays. Any detectable label known in the art may be used. In some examples the detectable label is selected from among a fluorophore, a fluorogenic substrate, a luminogenic substrate, and a biotin.

A fluorescent tag may be a fluorophore. For example, a fluorophore may be fluorescein isothiocyanate, fluorescein thiosemicarbazide, rhodamine, Texas Red, a CyDye such as Cy3, Cy5 and Cy5.5, a Alexa Fluor such as Alexa488, Alexa555, Alexa594 and Alexa647) or a near infrared fluorescent dye. A fluorophore may be a pH-sensitive fluorescent probe. For example, a pH-sensitive fluorescent probe may be naphthofluorescein, A fluorescent tag may be a fluorescent protein. For example, a fluorescent protein may be green fluorescent protein (GFP), enhanced green fluorescent protein (EGFP), AcGFP or TurboGFP, Emerald, Azami Green, ZsGreen, EBFP, Sapphire, T-Sapphire, ECFP, mCFP, Cerulean, CyPet, AmCyanl, Midori-Ishi Cyan, mTFPl (Teal), enhanced yellow fluorescent protein (EYFP), Topaz, Venus, mCitrine, YPet, PhiYFP, ZsYellow1, mBanana, Kusabira, ange, mOrange, dTomato, dTomato-Tandem, AsRed2, mRFP1, Jred, mCherry, HcRed1, mRaspberry, HcRed1, HcRed-Tandem, mPlum, AQ 143. A fluorescent tag may be a quantum dot. In some examples, where the detectable label is a fluorophore, the fluorophore is a pH-sensitive fluorescent probe. Suitable pH-sensitive fluorescent probes include, but are not limited to, naphthofluorescein, pHrodo™ Green (ThermoFisher), and pHrodo™ Red (ThermoFisher). Fluorescent tags may be detected using fluorescent microscopes such as epifluorescence or confocal microscopes, fluorescence scanners such as microarray readers, spectrofluorometers, microplate readers and/or flow cytometers.

In some examples the detectable label is a fluorogenic substrate. Suitable fluorogenic substrates include fluorogenic substrates of β-lactamase (e.g, CCF-2-AM, CCF4-AM, and any of those described in U.S. Pat. No. 7,427,680) and β-gal (e.g, HMRef-βGal described in Asanuma et al 2015, Nature Comm., 6:6463).

In some examples the detectable label is a luminogenic substrate. Suitable luminogenic substrates include, but are not limited to, D-Luciferin, L-Luciferin, Coelenterazine,

An epitope tag may be a poly-histidine tag such as a hexahistidine tag or a dodecahistidine, a FLAG tag, a Myc tag, a HA tag, a GST tag or a V5 tag. Epitope tags are routinely detected with commercially available antibodies. A person skilled in the art will be aware that an epitope tag may facilitate purification and/or detection. For example, a conjugate containing a hexahistidine tag may be purified using methods known in the art, such as, by contacting a sample comprising the protein with nickel-nitrilotriacetic acid (Ni-NTA) that specifically binds a hexahistidine tag immobilized on a solid or semi-solid support, washing the sample to remove unbound protein, and subsequently eluting the bound protein. Alternatively, or in addition a ligand or antibody that binds to an epitope tag may be used in an affinity purification method.

An isobaric tag may be a mass tag or an isobaric tag for relative absolute quantification (iTRAQ). A mass tag is a chemical label used for mass spectrometry based quantification of proteins and peptides. In such methods mass spectrometers recognise the mass difference between the labeled and unlabeled forms of a protein or peptide, and quantification is achieved by comparing their respective signal intensities as described, for example, in Bantscheff et al. 2007. Examples of mass tags include TMTzero, TMTduplex, TMTsixplex and TMT 10-plex. An isobaric tag for relative absolute quantification (iTRAQ) is a chemical tag used in quantitative proteomics by tandem mass spectrometry to determine the amount of proteins from different sources in a single experiment as described, for example, in Wiese et al. 2007.

In some examples the moiety is a non-canonical amino acid. Suitable non-canonical amino acids include, but are not limited to, ornithine, citrulline (Cit), diaminobutyric acid (Dab), diaminopropionic acid (Dap), 2-Aminoisobutyric acid α-Amino-n-butyric acid, Norvaline, Norleucine, Alloisoleucine, t-leucine, Ornithine, Allothreonine, β-Alanine, β-Amino-n-butyric acid, N-isopropyl glycine, Isoserine, and Sarcosine.

In other examples a moiety in a modified CPP is a reactive group. Suitable reactive groups include, but are not limited to, azide groups, amine-reactive groups, thiol-reactive groups, and carbonyl-reactive groups. In some examples the reactive groups are part of a chemical tag. Suitable chemical tags include, but are not limited to, a SNAP tag, a CLIP tag, a HaloTag or a TMP-tag. In one example, the chemical tag is a SNAP-tag or a CLIP-tag. SNAP and CLIP fusion proteins enable the specific, covalent attachment of virtually any molecule to a protein or peptide of interest as described, for example, in Correa 2015 (Methods Mol Biol, 1266:55-79). In another example, the chemical tag is a HaloTag. HaloTag involves a modular protein tagging system that allows different molecules to be covalently linked, either in solution, in living cells, or in chemically fixed cells. In another example, the chemical tag is a TMP-tag. TMP-tags are able to label intracellular, as opposed to cell-surface, proteins with high selectivity.

In some examples the moiety in a modified CPP is a fatty acid. Suitable fatty acids for modified peptides include, but are not limited to, palmitic acid, myristic acid, caprylic acid, lauric acid, n-octanoic acid, and n-decanoic acid.

In other examples the moiety in a modified CPP is cholesterol.

In some examples, where the moiety on a modified CPP is a polynucleotide, the polynucleotide is an RNAi, an antisense RNA, a single stranded DNA or RNA oligonucleotide, a double stranded DNA oligonucleotide, an mRNA, or a plasmid.

In some examples the moiety of a modified CPP is covalently linked to an amino acid in the CPP. In one example the covalently linked moiety is covalently linked to the N-terminal of the CPP amino acid sequence. In another example the covalently linked moiety is covalently linked to the C-terminal of the CPP amino acid sequence. In other examples, the covalently linked moiety is covalently linked through an amino acid residue side chain (e.g, at an internal lysine or cysteine residue). In some examples the moiety is non-covalently linked to the CPP, e.g, via non-covalent interactions between one or more charged amino acid residues in the CPP and one or more functional groups in the moiety that are of opposite charge to the one or more CPP amino acid residues.

In some examples the moiety in a modified CPP is a D-amino acid. In some examples, the amino acid sequence of a CPP is the retro-inverso sequence of the amino acid sequence of any of the foregoing CPPs.

Also described herein are CPP fusion proteins comprising the amino acid sequence of any CPP described herein, including a modified CPP and a heterologous amino acid sequence, Le, an amino acid sequence that is not naturally found as a sequence that is contiguous with the amino acid sequence of a CPP. In some examples, the heterologous amino acid sequence comprises the amino acid sequence of a protein selected from the group consisting of a SpyTag protein (SEQ ID NO:84), a Phylomer™ as defined herein, a reporter protein, a pro-apoptotic peptide, a targeting protein, a cytotoxic protein, a bioactive peptide, a dominant negative peptide, an enzyme, an antibody, and a SpyC peptide (SEQ ID NO:83). Examples of bioactive peptides include, but are not limited to, Glucagon (GCG), Glucose-dependent insulinotropic peptide (GIP, Cholecystokinin B (CCKB), Glucagon-like peptide 2 (GLP-2), as described in, e.g., Fosgerau et al (2015), Peptide therapeutics: current status and future directions, 20(1):122-128. Examples of suitable enzymes (e.g., therapeutic enzymes) include, but are not limited to, Acid Sphingomyelinase, Glucocerebrosidase, and I-L-Iduronidase.

In some examples the heterologous amino acid sequence in the CPP fusion protein is that of a SpyTag peptide (SEQ ID NO:84), which allows covalent isopeptide bond formation between the CPP fusion protein and a SpyCatcher protein as described in Zakeri et al 2012 (PNAS-USA, 109(12):E690-697). In other examples the CPP fusion protein comprises the amino acid sequence of a SpyTag peptide (SEQ ID NO:84), which is referred to herein as a CPP-SpyTag fusion protein.

In some examples a CPP-SpyTag fusion protein comprises the amino acid sequence of any one of SEQ ID NOs:42-60, 97, or 168-222.

In some examples the heterologous amino acid sequence comprises the amino acid sequence of a dominant negative peptide, e.g., the amino acid sequence of Omomyc (SEQ ID NO:99). In other examples the heterologous amino acid sequence comprises the amino acid sequence of a proapoptotic peptide. In some examples the amino acid sequence of the proapoptotic peptide comprises the amino acid sequence of SEQ ID NO:61 or SEQ ID NO:63. In other examples the heterologous amino acid sequence comprises the amino acid sequence of an enzyme. In some examples the enzyme is a therapeutic enzyme. In some examples the reporter protein comprises the amino acid sequence of β-lactamase (SEQ ID NO:112).

In some examples, the CPP fusion protein comprises a flexible linker linking the CPP and the heterologous amino acid sequence. Examples of flexible linkers include, but are not limited to, GGGGS (SEQ ID NO:262), GGGGSGGGGS (SEQ ID NO:263), GAS, GGG, GSG, GTG, and GGTAGSTGG (SEQ ID NO:264). Other examples of such flexible linkers are known in the art as described in, e.g., Chen et al (2013), Adv Drug Deliv Rev., 65(10):1357-1369.

Also described herein is a CPP conjugate comprising a CPP-SpyTag peptide fusion protein comprising the amino acid sequence of any Phylomer™-derived CPP disclosed herein and a SpyCatcher fusion protein comprising the amino acid sequence of SEQ ID NO:83 and a heterologous amino acid sequence, wherein the SpyCatcher fusion protein is covalently linked to the CPP fusion protein by an isopeptide bond to the SpyTag peptide. One of skill in the art will appreciate that such conjugates are readily generated by reacting a CPP fusion protein with a SpyCatcher fusion protein, whereby the SpyTag peptide and SpyCatcher peptide react with each other to form an amide bond as described in Zakeri, supra. Conveniently, such CPP conjugates allow the modular functionalization of a CPP with various peptides or proteins thereby avoiding the need to separate CPP fusion proteins with different functionalities (e.g, a CPP-β-lactamase fusion protein, a CPP-fluorescent protein fusion protein, etc.

In some examples the heterologous amino acid sequence in the above-mentioned CPP fusion proteins or conjugates is the amino acid sequence of a Phylomer™, a reporter protein, a pro-apoptotic peptide, an enzyme (e.g., Caspase-9), a targeting protein (e.g, a receptor affibody such as an EGFR affibody), a cytotoxic protein (e.g., Bouganin), a dominant-negative peptide (e.g, Omomyc, SEQ ID NO:99), an antibody, or a SpyC peptide (SEQ ID NO:83).

In some examples the heterologous amino acid sequence is the amino acid sequence of a Phylomer™.

In other examples the heterologous amino sequence is a reporter protein. Suitable reporter proteins include a fluorescent protein as described herein, a β-lactamase as described in Qureshi (2007), Biotechniques, 42(1):91-95, a haloalkane dehalogenase, or a luciferase. In some examples the reporter protein comprises the amino acid sequence of a β-lactamase.

In some examples the heterologous amino acid sequence is the amino acid sequence of a pro-apoptotic peptide. In some examples the amino acid sequence of the pro-apoptotic peptide corresponds to SEQ ID NO:61 or SEQ ID NO:63. In some examples a SpyCatcher-pro-apoptotic peptide fusion protein in the CPP conjugate comprises the amino acid sequence of SEQ ID NO:62.

In some examples the heterologous amino acid sequence is a targeting moiety. A targeting moiety may provide increased specificity to CPP conjugates by binding to a specific cell surface antigen (e.g, a receptor), which is then internalized into endosomes. The CPPs disclosed herein can provide the added advantage relative to conventional CPPs of increased escape from endosomes and enhanced cytosolic delivery of conjugate “cargoes.” Examples of targeting proteins include, but are not limited to, Affibodies, scFvs, single chain antibodies, and other selective binding proteins using alternative scaffolds (e.g, peptide aptamers). In some examples the targeting moiety is an EGFR affibody. In other examples the heterologous amino acid sequence is a cytotoxic protein (e.g, Bouganin or diphtheria toxin) that induces rapid cell death upon internalization and escape from endosomes.

In some examples the heterologous amino acid sequence is a dominant negative peptide. Dominant negative peptides generally act to interfere with one or more functions of a protein from which they are derived and/or with that of an interacting partner of the full length protein. Typically, they act by interfering with the interaction of a protein and one or more of its binding partners. In some examples the dominant negative transcription factor peptide is an anti-cancer peptide. Suitable anti-cancer peptides include, but are not limited to, Omomyc (SEQ ID NO:99), an Activating Transcription Factor 5 (ATF5) dominant negative peptide d/n-ATF5-S1 (SEQ ID NO:85) described in Massler et al (2016), Clin Cancer Res, 22(18):4698-4711, anti-Ras-p21 dominant negative peptides such as ras-p21 96-110 (PNC-2) (SEQ ID NO:86) and ras-p21 35-47 (SEQ ID NO:87) as described in Adler et al (2008), Cancer Chemother Pharmacol, 62(3):491-498. In other examples, the heterologous amino acid sequence is an enzyme. In some examples the enzyme is a genomic targeting protein (e.g, a CRISPR-associated protein 9/Cas9 genomic targeting protein or a Cpf1 genomic targeting protein). In other examples the enzyme is a caspase (e.g., Caspase-9).

Any protein or peptide of the present disclosure may be synthesized using a chemical method known to the skilled artisan. For example, synthetic proteins and peptides are prepared using known techniques of solid phase, liquid phase, or peptide condensation, or any combination thereof, and can include natural and/or unnatural amino acids.

Any protein of the present disclosure may be expressed by recombinant means. For example, the nucleic acid encoding the CPP may be placed in operable connection with a promoter or other regulatory sequence capable of regulating expression in cellular system or organism.

Typical promoters suitable for expression in bacterial cells include, for example, the lacz promoter, the Ipp promoter, temperature-sensitive μ_{L} or λ_{R} promoters, T7 promoter, T3 promoter, SP6 promoter or semi-artificial promoters such as the IPTG-inducible tac promoter or lacUV5 promoter. A number of other gene construct systems for expressing the nucleic acid fragment of the invention in bacterial cells are well-known in the art and are described, for example, in Ausubel et al. (1988), and Sambrook et al. (2001).

Numerous expression vectors for expression of recombinant polypeptides in bacterial cells have been described, and include, for example, PKC3, pKK173-3, pET28, the pCR vector suite (Invitrogen), pGEM-T Easy vectors (Promega), the pL expression vector suite (Invitrogen) or pBAD/thio—TOPO series of vectors containing an arabinose-inducible promoter (Invitrogen), amongst others.

Typical promoters suitable for expression in yeast cells such as, for example, a yeast cell selected from the group comprising Pichia pastoris, S. cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Expression vectors for expression in yeast cells are preferred and include, for example, the pACT vector (Clontech), the pDBleu-X vector, the pPIC vector suite (Invitrogen), the pGAPZ vector suite (Invitrogen), the pHYB vector (Invitrogen), the pYD 1 vector (Invitrogen), and the pNMT 1, pNMT41, pNMT81 TOPO vectors (Invitrogen), the pPC86-Y vector (Invitrogen), the pRH series of vectors (Invitrogen), pYESTrp series of vectors (Invitrogen).

Preferred vectors for expression in mammalian cells include, for example, the pcDNA vector suite (Invitrogen), the pTARGET series of vectors (Promega), and the pSV vector suite (Promega).

Suitable methods for transforming and transfecting host cells can be found in Sambrook et al. 2001 and other laboratory textbooks. In one example, nucleic acid may be introduced into prokaryotic cells using for example, electroporation or calcium-chloride mediated transformation. In another example, nucleic acid may be introduced into mammalian cells using, for example, microinjection, calcium phosphate or calcium chloride co-precipitation, DEAE-dextran mediated transfection, transfection mediated by liposomes such as by using Lipofectamine (Invitrogen) and/or cellfectin (Invitrogen), PEG mediated DNA uptake, electroporation, transduction by Adenoviuses, Herpesviruses, Togaviruses or Retroviruses and microparticle bombardment such as by using DNA-coated tungsten or gold particles. Alternatively, nucleic acid may be introduced into yeast cells using conventional techniques such as, for example, electroporation, and PEG mediated transformation.

Following production/expression/synthesis, any protein or peptide of the present disclosure can be purified using a method known in the art such as HPLC See e.g, Scopes (In: Protein purification: principles and practice, Third Edition, Springer Verlag, 1994).

The present disclosure provides a method of identifying a peptide capable of translocating a membrane of a cell comprising:
i) contacting a cell with a CPP; and
ii) detecting the CPP in the cell.

A CPP may be modified to facilitate detection. For example, a CPP may be linked to a detectable label, such as naphthofluorescein (CAS No. 61419-02-1; λₑₓ=594 nm, λₑₘ=663 nm). Naphthofluorescein is a pH sensitive fluorophore that ranges from non-fluorescent at pH≤5.5 to maximal fluorescence at pH≥9.0, with 50% fluorescence intensity at pH≈7.5. Such pH sensitive fluorophores are advantageous because they are non-fluorescent in the acidic endosomal or lysosomal environment but become fluorescent when released into the neutral cytosol. Therefore, pH sensitive fluorophores may be conjugated to a CPP to measure its ability to escape the endosome. For convenience, CPPs that can not only enter cells, but can also escape endosomal or lysosomal compartment can be referred to as “functional penetrating peptides” (FPPs).

Alternatively, the ability of a peptide to not only translocate a membrane, but also to escape an endosomal compartment can be assessed using a phenotypic endpoint that discriminates selectively identifies CPPs that are localized in the cytoplasm (FPPs) and not entrapped in an endosomal compartment. For example, the ability of a test CPP-pro-apoptotic peptide conjugate to be delivered to target cells can be assessed by measuring cell death of the target cells following contact with the test CPP-pro-apoptotic peptide conjugate versus the level of cell death following contact with the unconjugated pro-apoptotic peptide or CPP peptide separately. Alternatively, a CPP conjugated to a pH-sensitive fluorescent probe (e.g., naphthofluorescein) can be used to discriminate between CPP localization to acidic endosomal/vesicular compartments versus the neutral cytoplasm.

Also described herein is a modified cell comprising any of the CPPs, CPP fusion proteins, or CPP conjugates described herein. In some examples a modified cell is a prokaryotic cell. In other examples the modified cell is a eukaryotic cell. Suitable eukaryotic cells include yeast cells, and mammalian cells including, but not limited to human cells. In some examples modified mammalian cells are from a cell line. Suitable cell lines include, but are not limited to, CHO-K1, HEK-293, COS7, HeLa, N2a, and NIH 3T3.

In some examples a modified cell expresses one or more genetically encoded CPPs or CPP fusion proteins. In other examples a modified cell is a primary mammalian cell.

In other examples a modified cell does not comprise exogenous nucleic acids encoding a CPP or CPP fusion protein, but is modified by protein transduction of a CPP or CPP fusion protein.

Preferably the modified cells are eukaryotic cells. More preferably the eukaryotic cells are mammalian cells. Most preferably the mammalian cells are human cells. In some examples the human cells are human stem cells. Such human stem cells include, but are not limited to, embryonic stem cells, induced pluripotent stem cells, and mesenchymal stem cells. In further examples human cells include, but are not limited to, cardiomyocytes, neurons, hepatocytes, and pancreatic islet cells. In other examples, the mammalian cells are cancer cells (e.g., human cancer cells).

The present disclosure also provides any one of the CPPs, CPP fusion proteins, CPP conjugates, or modified cells for use as a medicament or diagnostic agent. The present disclosure also provides any one of the CPPs, CPP fusion proteins, CPP conjugates, or modified cells for use in the manufacture of a medicament or diagnostic agent.

The present disclosure also provides a method for delivering any of the CPPs, CPP fusion proteins, or CPP conjugates disclosed herein to a cell by contacting the cell with any of these. In some examples the contacting is performed ex vivo, e.g., in cultured eukaryotic cells. In other examples the contacting is performed in vivo, e.g., in a human subject.

The invention will now be further described with reference to the following, non-limiting examples.

### EXAMPLES

### Example 1: Measurement of Peptide Cell Penetration and Endosomal Escape by Apoptosis Assay

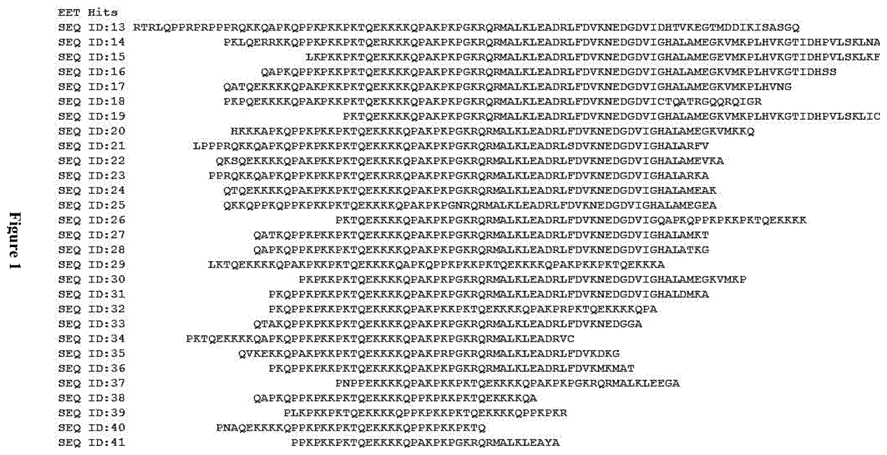

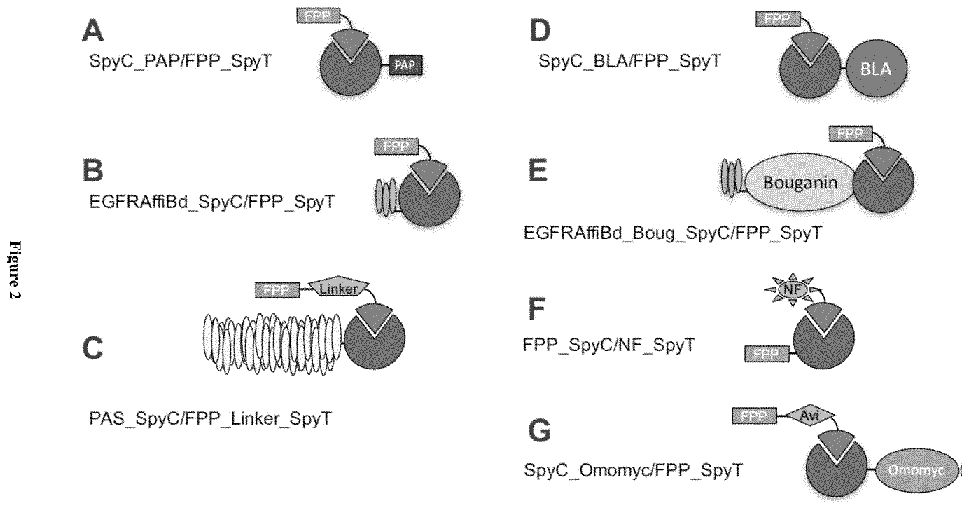

We previously identified a series of Phylomer™ sequences in a genetic screen designed to enrich for peptides not only able to penetrate eukaryotic cells, but also able to escape the endosomal and lysosomal compartments following uptake into cells. Such peptides are referred to here as “functional penetrating peptides” (FPPs) to distinguish them from CPPs that only penetrate the cell membrane but become entrapped in the endosomal or lysosomal compartments. This screen resulted in the identification of various Phylomer™ sequences, including a series of Sindbis capsid-derived peptides (corresponding to SEQ ID Nos:13-41 shown in FIG. 1). The Phylomer™ peptide corresponding to SEQ ID NO:39 was chosen as a candidate FPP (“FPP1”) and a starting point for FPP optimization. Our FPP optimization strategy focused on identifying the minimal peptide domain without compromising FPP activity, and assessing other modifications to increase potency. To facilitate this, we developed a modular approach based on the SpyCatcher/SpyTag protein ligation technology (Zakeri et al 2012, Proc. Natl. Acad. Sci USA, 109, E690-697). SpyTag (SpyT; SEQ ID NO:84) is a short peptide sequence that forms an isopeptide bond with the SpyCatcher (SpyC; SEQ ID NO:83) partner protein in an irreversible peptide-protein coupling. We synthesized variants of FPP1 fused to SpyTag (SpyT), which enabled us to couple FPP1 and variants to any cargo protein or peptide that is fused to the SpyCatcher (SpyC) protein (see FIG. 2 for illustrations of the SpyC/SpyT conjugates used in these examples). The main focus was on delivering cargoes with functional readout dependent on endosomal escape of the Phylomer-delivered proteins.

### Methods for Example 1 and Subsequent Examples

### Peptide Constructs:

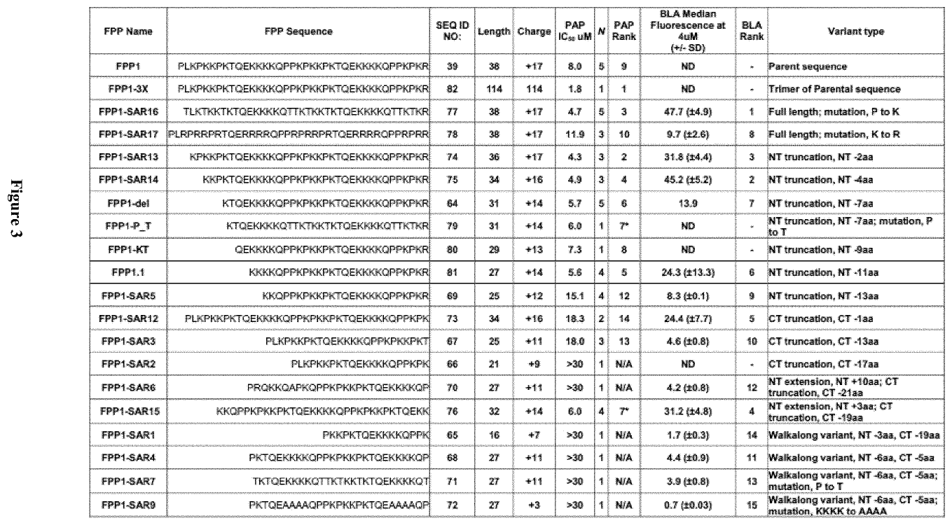

CPP sequences, (derived from parental sequences corresponding to SEQ ID NOs:13-41, and encompassing N-terminal truncations, C-terminal truncations, truncations, deletions, point and contiguous sequence mutations and all variations thereof), were synthesized by Pepscan (Netherlands) and Mimotopes (Australia) as fusion proteins N-terminal to the SpyTag sequence (SEQ ID NOs:42-60). The CPP sequences used in the fusion proteins are shown in FIG. 3 (SEQ ID NOs:39 and 65-82).

### Protein Constructs:

pET28a+ SpyCatcher-PAP (SpyC-PAP; SEQ ID NO:62) was codon optimized for E. coli expression and synthesized (DNA 2.0, Menlo Park, Calif., USA). The synthesised cassette is cloned into the NcoI/XhoI of the pET28a+ expression vector (Novagen). The cassette includes a hexahistidine tag and prescission protease cleavage site to aid purification. This cassette comprises the SpyCatcher sequence (SEQ ID NO:83) and the 14 amino acid PAP sequence (SEQ ID NO:61).

### Recombinant Protein Expression and Purification

DNA sequences were synthesized and cloned (ATUM) into NcoI and XhoI sites of pET28a⁺ vector (Merck Millipore). Recombinant proteins were expressed as His₆-N-terminally tagged fusion proteins in E. coli strain BL21 (DE3) Gold (Agilent Technologies). Proteins were purified using IMAC as previously described (Milech et al 2015, Sci Rep 5, 18329) with an additional purification step performed for some proteins after IMAC using Ion Exchange Chromatography (IEX). Proteins with an isoelectric point (pI) ranging from 8 to 10 were desalted into binding buffer containing 20 mM Sodium Phosphate, pH 6.8. Samples were loaded on a HiTrap SP HP 5 mL column (GE Healthcare), and eluted with a 0-1M. NaCl gradient. All the other proteins were desalted into 20 mM Tris, pH 8.0 binding buffer and purified through a HiTrap Q HP 5 mL column (GE Healthcare). Proteins were eluted using a 0-1M NaCl gradient. Final proteins were desalted into PBS pH 7.4, and purity was confirmed by analysis on 4-16% SDS-PAGE stained with Gel Code Blue Reagent. Recombinant Omomyc proteins were expressed and purified similarly by the UQ Protein Expression Facility (University of Queensland, Australia).

Recombinant His_SpyC_BLA (SEQ ID NO:90) and His_SpyC_BLA_FPP1.1 (SEQ ID NO:91) proteins were made as previously described (Milech, supra) with the following modifications: Pellets from 100 ml cultures grown at 28° C. for 18 h were purified by the IMAC gravity flow protocol with 1 ml of Ni Sepharose High Performance slurry. To avoid precipitation due to the high protein yield, the eluted proteins were diluted 1:4 in a buffer containing 20 mM phosphate, 500 mM NaCl and 20 mM imidazole, pH 8.0 and dialysed slowly (SnakeSkin Pleated Dialysis Tubing, 7,000 MWCO; Thermo Scientific) against 50 mM Tris pH 7.5, 200 mM NaCl buffer overnight at 4° C. The protein solutions were sterile-filtered (0.22 μm) and concentrated (Amicon Ultra-15, MWCO 10K; Merck Millipore).

SpyC-free Omomyc recombinant proteins were expressed with an N-terminal Thioredoxin (Trx) solubility tag, containing an His₆_HRV3C (Human Rhinovirus 3C protease) cleavage sequence positioned at the C-terminus of Trx. After IMAC purification, proteins were desalted into IEX buffer, followed by tag removal using HRV3C enzymatic digestion overnight at 4° C. with agitation. Digested samples were further purified using IEXC, desalted into PBS pH 7.4, and analyzed as described above. SpyC_PAS protein, where SpyC is expressed as a recombinant fusion protein with PAS (PAS sequence described in Schlapsky et al. 2013, Protein Engineering, Design & Selection, 26(8):489-501) was provided by Professor Arne Skerra, Technical University of Munich, Munich, Germany.

### SpyCatcher/SpyTag Conjugations

With the exception of SpyC PAS proteins, conjugations were set up at a SpyCatcher:SpyTag ratio of 1:1.25, with a 40 μM final concentration for the SpyCatcher moiety. SpyC proteins and SpyTag peptides were incubated for 2 h at 22° C. with gentle mixing, and then left at 4° C. overnight. Conjugation efficiencies were analyzed on 4-16% SDS-PAGE gels stained with Gel Code Blue Reagent (Thermo Fisher Scientific). SpyC_PAS proteins were conjugated with SpyTag peptides at ratio 1:1.1, mixed and incubated at room temperature for 30 mins before being stored overnight at 4° C.

### Mammalian Cell Culture

All cell lines were maintained in a humidified incubator at 37° C. with 5% CO₂. HEK-293 and A431 cells were cultured in DMEM supplemented with 10% FCS, 2 mM L-glutamine, 100 U/ml penicillin, and 100 μg/ml streptomycin. CHO-K1, T47D and AMO-1 cells were cultured in RPMI 1640 supplemented with 10% FCS (heat-inactivated), 2 mM L-glutamine or 2 mM Glutamax (LifeTech), 100 U/ml penicillin, and 100 μg/ml streptomycin. HL-60 cells were cultured in RPMI 1640 supplemented with 20% FCS (heat-inactivated), 100 U/ml penicillin, 100 μg/ml streptomycin, 2 mM L-glutamine. Stable cell lines HEK-293 EGFR and HEK-293_BirA were cultured in HEK-293 complete medium additionally supplemented with 300 μg/ml and 500 μg/ml Geneticin, respectively. CHO-K1 EGFR stable cell line was made using FlpIn™ technology (ThermoFisher Scientific) and cultured in F-12K medium supplemented with 10% FCS (heat-inactivated), 100 U/ml penicillin, 100 μg/ml streptomycin, 2 mM L-glutamine and 800 μg/ml Hygromycin B. Stable cell line A431_BirA (made by lentiviral infection, Genscript) was cultured in A431 complete medium additionally supplemented with 1 μg/ml Puromycin.

### Cell Culture for Biotinylation Experiments

HEK-293_BirA cells were electro-transfected with plasmid DNA (pcDNA4/TO_β-actin, pcDNA4/TO_β-actin_AviTag) using the Neon transfection system (LifeTech). Briefly 3×10⁵ cells/100 μL were prepared according to manufacturer's instructions (Neon, LifeTech) and transfected with 0.5 μg DNA with the following pulse conditions: 1100 V, 20 ms, 2 pulses. Following transfection cells were transferred to 8 well glass chamber slides (Nalgene Nunc International) pre-coated with gelatin and were maintained in media+/−5 μM biotin for 18 h. Media was changed to biotin-free media for 2 h prior to processing to reduce non-specific background. At 20 h post transfection cells were fixed (4% formaldehyde/PBS) and permeabilized (0.4% Saponin/PBS) for 1 h at 4° C. Successful biotinylation of the AviTag was detected by incubation with Streptavidin-FITC (1:100 in the permabilization buffer) for 60 min. For wells that were counterstained a second incubation with β-actin red (LifeTech) was performed following manufacturer's instructions. Wells were then washed and mounted with Vectashield antifade mounting media with DAPI (Vector Laboratories, USA). Cell images were captured at 100× magnification using an Olympus BX53 with DP72 camera. Image overlays were compiled using the ImageJ software.

### Detection of Biotinylated Avitagged Protein Conjugate Inside Cells

Peptide/protein conjugates were prepared in parallel, with SpyC moieties at 40 μM concentration and the SpyT peptides at 50 μM concentration. Conjugations were incubated at room temperature for 1 h and then buffers and BirA ligase (2.9 ng/μl final concentration) were spiked into one tube for in vitro biotinylation. All conjugations were then incubated overnight at 4° C. in the cold room with gentle rotation.

HEK-293_BirA cells were seeded in 6-well plates (8×10⁵ cells/well) and incubated overnight. The following day, cells were treated with either FPP1.1_Avi_SpyT (SEQ ID NO:111)/SpyC_Omomyc (SEQ ID NO:110) conjugate or in vitro biotinylated conjugate for 30 mins or 60 mins. Treated samples were then lysed with M-PER mammalian protein extraction reagent (Thermo Fisher Scientific), supplemented with 1× cOmplete protease inhibitors cocktail (Sigma) and 1 mM sodium pyrophosphate (BirA inhibitor, Sigma). Supernatants were clarified by centrifugation (10,000 rpm, 20 s). Clarified lysates (˜800 μl each sample) were then incubated with 40 μl of 1:1 slurry of washed Dynabeads™ M-280 streptavidin magnetic beads (Thermo Fisher Scientific) in PBS overnight with gentle rotation at 4° C. according to manufacturer's instructions, to bind any biotinylated proteins in the samples. The following day, bead samples were washed and proteins denatured in Laemmli buffer according to manufacturer's instructions.

### Immunoblotting

Proteins were separated on Bis-Tris gels (Thermo Fisher Scientific) by SDS-PAGE and transferred to PVDF membranes by iBLOT (Thermo Fisher Scientific). Immunoblots were processed as previously described, (Milech, supra) using primary and secondary antibodies according to manufacturer's instructions.

For detection of V5-tagged protein conjugates and peptides, immunoblots were probed with anti-V5 primary antibody (Thermo Fisher Scientific, clone E10) and secondary anti-Mouse-HRP antibody (Amersham) before visualizing using Clarity ECL reagent (BioRad) and imaged on a ChemiDoc Gel Imaging System (BioRad).

### Cell Viability Assay

Cells in were seeded at 2000-6000 cells/well, depending on cell line, in 96-well plates (PAP assays: CHO-K1, 3000 cells/well; peptide cytotoxicity assays: CHO-K1, 5000 cells/well; Bouganin assay: CHO-K1 and CHO-K1 EGFR, 2500 cells/well; ^{D}PMIα assays: T47D, 5000 cells/well; Omomyc assays: all cell lines, 5000 cells/well). In brief, adherent cells were allowed to attach for 24 h prior to addition of treatments whereas suspension cell lines were treated immediately following seeding. Following 2-48 h incubations with treatments, cell viability was measure by a variety of methods. Membrane integrity was assessed by the release of LDH into the media via the CytoTox-ONE reagent (Promega). Metabolic activity was measured either by resazurin reduction potential using PrestoBlue (LifeTech) or ATP activity using CellTitre-Glo (Promega). All assays followed manufacturer's instructions. IC50 values were calculated using Prism (version 7.0a, GraphPad).

### Cell Penetrating Peptide Ranking:

SpyC-PAP/CPP-SpyT complex IC50s were ranked according to potency to determine impact of changes to primary CPP sequence. Sequence variations positively impacting on potency are retained for further optimization, whereas sequence variations deleterious to potency are excluded, thus establishing sequence activity relationships for the CPP peptide. SpyC_BLA/CPP-SpyT complex median cell fluorescence at 4 μM concentration was ranked according to intensity to determine impact of changes to primary CPP sequence. Sequence variations positively impacting on intensity were retained for further optimization, whereas sequence variations deleterious to intensity were excluded, thus establishing sequence activity relationships for the CPP peptide.

### β-Lactamase (BLA) Bioassay

CHO-K1 cells (seeded at 1×10⁵ cells/well in 24-well plates) were incubated with purified SpyC_BLA (SEQ ID NO:90) and SpyC_BLA_FPP1.1 (SEQ ID NO:91) proteins at 3° C./5% CO₂ for 2 hours. Cells were washed, detached by 5 mins incubation with trypsin, washed, loaded with the β-lactamase substrate CCF2-AM and analysed by Flow Cytometry; intracellular β-lactamase activity caused an emission shift from 510 nm to 450 nm. The percentages of β-lactamase positive cells for each sample were graphed against the concentration of protein added to the cells.

### Phosphorodiamidate Morpholino Oligomer (PMO) Exon-Skipping Assay

Exon skipping assays and RT-PCR detection were performed according to published protocols (Morgan et al 1994, Developmental Biology, 162, 486-498) treating murine H-2K^{b}-tsA58 myoblast cells with 25 nM-1 μM of FPP1.1_M23D(+7-18) PMO or M23D(+7-18) PMO (Mann et al 2002, Gene Med, 4, 644-654) alone.

### Systemic Delivery of PMOs

Animal experiments and the detection of dystrophin expression by immunofluorescence microscopy were carried out according to published protocols (Fletcher et al 2007, Mol. Ther., 15, 1587-1592). Mice were treated with five intra-peritoneal injections, at 4 nmoles per dose, over two weeks of FPP1.1_M23D(+7-18) PMO or M23D(+7-18) PMO alone. Each treatment group consisted of two animals. Two weeks after cessation of treatment, tissue samples were taken for detection of dystrophin by immunofluorescence.

C57BL/10ScSnArc^{mdx} mice carry a nonsense mutation in exon 23 of the dystrophin gene. Control wild type mice are C57BL10/ScSnArc. All mice were supplied by the Animal Resources Centre (Murdoch, Western Australia) and housed according to National Health and Medical Research Council (Australia) guidelines. All animal work was approved and carried out under Murdoch University Animal ethics permit number R2625/13.

### Carboxynaphthofluoroscein (NF) Flow Cytometry

Flow cytometry and analysis of carboxynaphthofluoroscein (NF)-labeled peptides was performed according to published protocols (Qian et al 2015, Chem. Commun. (Camb), 51, 2162-2165; and Qian et al 2016, Biochemistry, 55, 2601-2612). Seeding density for the HEK-293_BirA cells was 3.6×10⁵ cells/well in 12-well plates. Peptides and cells were incubated in treatment media (phenol-red free high glucose DMEM, 1% FCS, 10 mM HEPES and 2 mM L-glutamine) unless otherwise specified.

### Endocytotic Inhibitor Assays

For endocytotic inhibitor assays, HEK-293_BirA cells were pre-treated with endocytotic inhibitors (100 μM dimethylamiloride (DMA, Abcam) or 20 μM Dyngo4a (Abcam) or vehicle control (1% DMSO in treatment media) for 30 min at 37° C. The washed cells were treated with 5 μM recombinant FPP1.1_SpyC protein conjugated to NF-LL1-SpyT (SEQ ID NO:94). For heparinase experiments, HEK-293_BirA cells were pre-incubated with 3 milli-Inhibitory Units (mIU) of heparinase III (Sigma) in DMEM containing 1% FCS for 1 h at 37° C. Then washed cells were treated with 5 μM FPP1.1_SpyC (SEQ ID NO:100) or SpyC (non-CPP control; SEQ ID NO:83) conjugated to NF-LL1-SpyT in serum-free media and incubated for 30 minutes at 37° C. For HSPG experiments, 5 μM FPP1.1_SpyC or SpyC were conjugated to NF-LL1-SpyT, then pre-incubated with 0, 5 or 10 μg/ml HSPG (Sigma) in serum free medium for 25 mins at 37° C. Then peptide-HSPG mixtures were added to HEK-293_BirA cells and further incubated for 30 minutes at 37° C. In all assays, peptide/protein conjugate uptake was measured by flow cytometry to detect the NF fluorescence signal.

### Results for Example 1

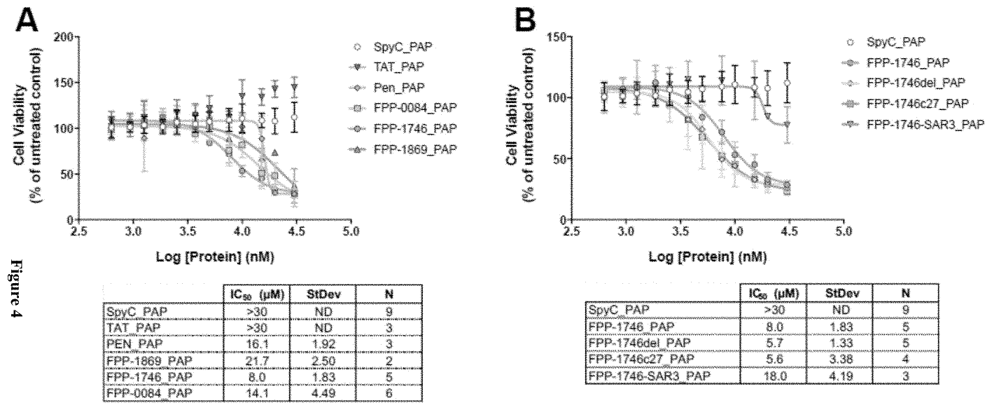

For the first readout of functional activity we chose the proapoptotic peptide PAP (SEQ ID NO:61; Ellerby et al 1999, Nat Med, 5, 1032-1038) as a functional cargo and expressed it recombinantly, fused to SpyCatcher (SEQ ID NO:83) (abbreviated as “SpyC_PAP” or “SpyC-PAP”; SEQ ID NO:62) to enable subsequent conjugation with synthetic CPP-SpyTag fusion proteins. Mammalian cells were incubated with SpyC_PAP-CPP protein conjugates, which induce apoptosis if successfully delivered into cells. SpyC-PAP conjugated with FPP1 (SEQ ID NO:39) caused significant cell death compared to TAT (SEQ ID NO:93) or Penetratin (SEQ ID NO:92)-delivered SpyC_PAP, with the SpyC only control showing no effect (FIG. 4A). As a representative member of the largest cluster of sequences from the screen, FPP1 was selected for additional optimization studies.

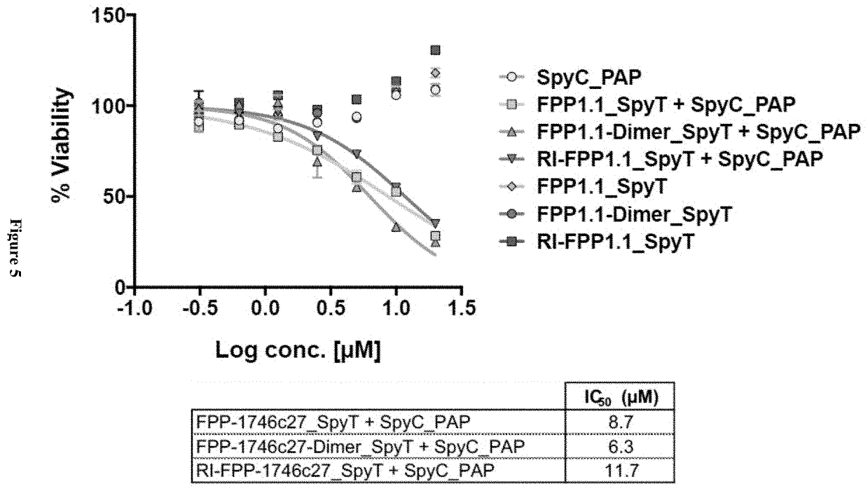

We designed a range of amino acid substitution variants and N- and C-terminal truncations to reduce the size and charge of the peptide, as well as sequence extensions based on larger members of the identified Sindbis Phylomer™ family and assessed the potency of these variants and the effect of the different truncations and sequence modifications. Overall, N-terminal truncations of parental FPP1 by up to 11 amino acids marginally improved potency, whereas an N-terminal reduction of 13 amino acids (FPP1-SAR5) resulted in a 3-fold loss of activity. In contrast, potency was unaffected for the N-terminal 7 amino acid truncated variant (FPP1-P_T). C-terminal truncations of FPP1 were deleterious to CPP potency, with even a single amino acid truncation (FPP1-SAR12) resulting in approximately 2.2-fold reduction. Mutation of Proline to Threonine (P to T) for full length FPP1 improved activity by 1.7-fold (FPP1-SAR16), whereas activity was unaffected for the N-terminal 7 amino acid truncated variant (FPP1-P_T). Mutation of Lysine to Arginine (K to R, FPP1-SAR17) resulted in a 1.5-fold reduction in potency. Taken altogether, these data suggest that the C-terminal arginine residue is critical for full activity, C-terminal truncation is deleterious, and that Proline is not essential for full potency but multiple Lysine residues are. The data also highlights that the C-terminal 27 residues (FPP1.1; SEQ ID NO:81) comprised the minimum domain sufficient for potent activity (FIGS. 3, and 4B—data shown for FPP1-del, FPP1.1, and FPP1-SAR3 variants only). Retro-inverso and FPP1.1 dimer-SpyTag (SEQ ID NO:101) fusions showed similar potency in an initial study, suggesting that both the natural and retro-inverso forms of FPP1.1 were active and stable for cargo delivery (FIG. 5).

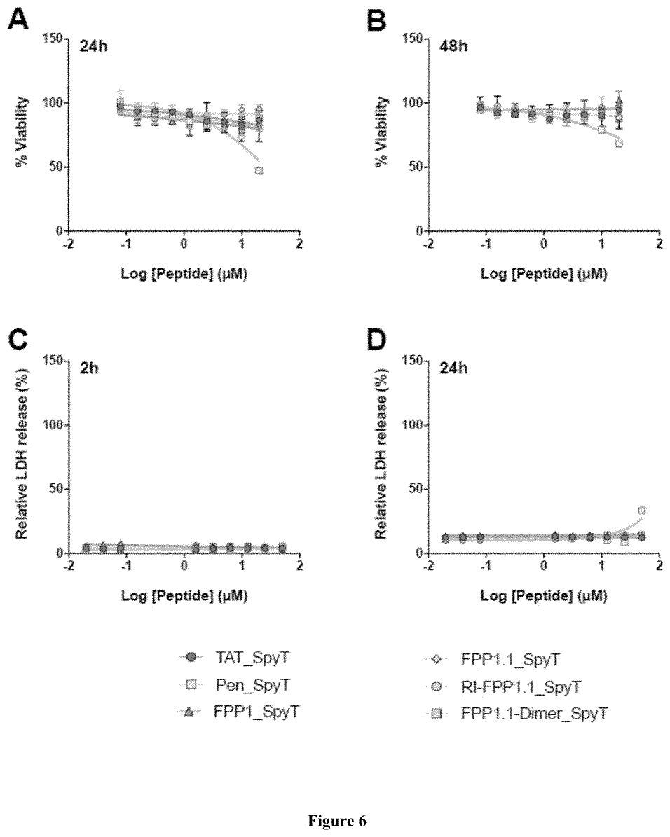

When delivering therapeutics into a cell, it is critical that the delivery molecule itself is not cytotoxic. To assess innate cytotoxicity we tested the effects of FPPs alone on cell viability at 24 h (FIG. 6A) and 48 h (FIG. 6B) after addition of peptides. The effects of FPPs on membrane stability of cells was also assessed in a LDH enzymatic assay, measuring release of LDH from cells into surrounding media at 2 h (FIG. 6C) and 24 h (FIG. 6D) after treatment with peptides. In all experiments, the Phylomer™-based FPPs showed no obvious cytotoxic potential up to the highest concentrations tested (50 μM). The canonical CPP TAT-SpyTag fusion (SEQ ID NO:95) also showed no cytotoxic activity, whereas Penetratin CPP-SpyTag fusion (SEQ ID NO:96) showed some inconsistent cytotoxicity at the highest concentrations in cell viability assays. Since the sequence variant FPP1.1 combined good activity improvement and necessary reduction in length and charge with lack of cytotoxicity, it was selected as our lead Phylomer FPP™ in subsequent experiments.

### Example 2: Measurement of Peptide Cell-Penetration and Endosomal Escape by a Fluorescent Enzymatic Assay

The method described here outlines the measurement of peptide cell-penetrating and endosomal escape ability by coupling it to an enzyme and measuring cytoplasmic enzyme activity, where increased enzyme activity is indicative of increased cell penetration and delivery of enzyme.

β-Lactamase is a bacterial enzyme that catalyses the opening of β-lactam rings. It does not occur naturally in eukaryotic cells. β-Lactamase is also not intrinsically cell-penetrating and requires the addition of a cell-internalising agent to access the eukaryotic cytoplasm. To address this need, β-Lactamase was expressed as C-terminal SpyC fusion, making SpyC-BLA (SEQ ID NO:90). Cell-penetrating peptides were then added by conjugating to various synthetic CPP-SpyT fusion peptides (SEQ ID NOs:42-60 and 102), and the SpyC-BLA was independently reacted with each CPP-SpyT peptide to be tested to form a CPP conjugate as schematically illustrated in FIG. 2B.

CCF2-AM (Thermofisher Scientific, Australia) is a Fluorescence Resonance Energy Transfer (FRET) substrate that is enzymatically cleaved by β-Lactamase. CCF2-AM is an esterified form of 7-hydroxycoumarin linked to fluorescein by a cephalosporin core. Esterification facilitates cell entry of the molecule. Once inside, the molecule is transformed into its anionic form by endogenous cytoplasmic esterases which trap the molecule inside the cell. When excited at 409 nm, uncleaved CCF2-AM emits a FRET signal at 520 nm (green). In the presence of β-Lactamase, the fluorescein moiety of CCF2 is enzymatically cleaved, resulting in the emission wavelength shifting to 447 nm (blue). β-Lactamase activity is quantified by measuring the ratio of blue fluorescence to green fluorescence.

The cell-penetrating and endosomal escape ability of FPP1.1 (SEQ ID NO:39) and sequence variant SpyC-BLA/CCP-SpyT conjugates was assessed by flow cytometry to determine cytosolic CCF2-AM cleavage by internalized β-Lactamase. The ratio of blue to green fluorescence was assessed to determine the cell-penetrating and endosomal escape ability of the various CPP sequences.

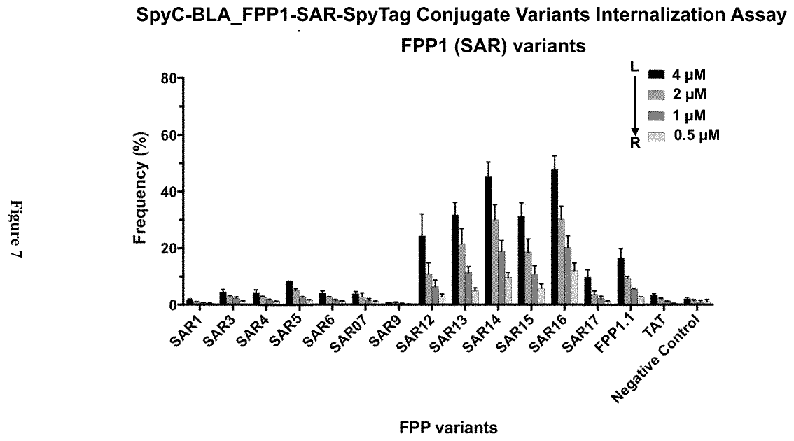

Various SpyC-BLA-FPP variant conjugates including CPPs corresponding to the parental sequence “FPP1” (SEQ ID NO:39), a derivative “FPP1.1” (SEQ ID NO:81), and other variants (SEQ ID NOs:64-80, and 82) all shown in FIG. 3 were tested for their ability to deliver β-lactamase to the cytosol at various doses. As shown in FIG. 7, a wide range of β-lactamase activity was observed for the FPP variants tested with some variants (e.g, FPP1.1) exhibiting almost 20-fold higher activity relative to unconjugated SpyC-BLA. In fact, all but two variants exhibited superior activity compared to a SpyC-BLA_TAT-SpyTag positive control conjugate. Two of the variants tested (FPP1-SAR1 and FPP-1) were ineffective in delivering BLA. These results showed that (BLA) cargo internalization was delivered in a dose-dependent manner down to 500 nM, and proved that the cargo was not only delivered but still functional.

### Example 3: Phylomer FPPs are Versatile and Potent Delivery Vehicles for a Range of Biologics In Vitro and In Vivo

We evaluated the versatility of several of the identified Phylomer™-based FPPs in a variety of cell types using bioassays where readout is dependent on delivery of a cargo into the cytoplasm. In the previous experiment, we demonstrated that the FPPs could deliver a functional protein cargo (i.e., active β-lactamase). Thus, we strategically chose a range of additional cargo types from biologic or therapeutically-relevant categories such as small peptides (^{D}PMIα, Liu et al 2010, Proc. Natl. Acad. Sci. USA, 107, 14321-14326) and oligonucleotides (M23D(+7-18), a phosphorodiamidate morpholino oligomer (PMO), Mann et al 2002, Gene Med, 4, 644-654). Overall, Phylomer™ FPPs were successful in delivering all three cargo types into cells at lower doses than conventional CPPs.

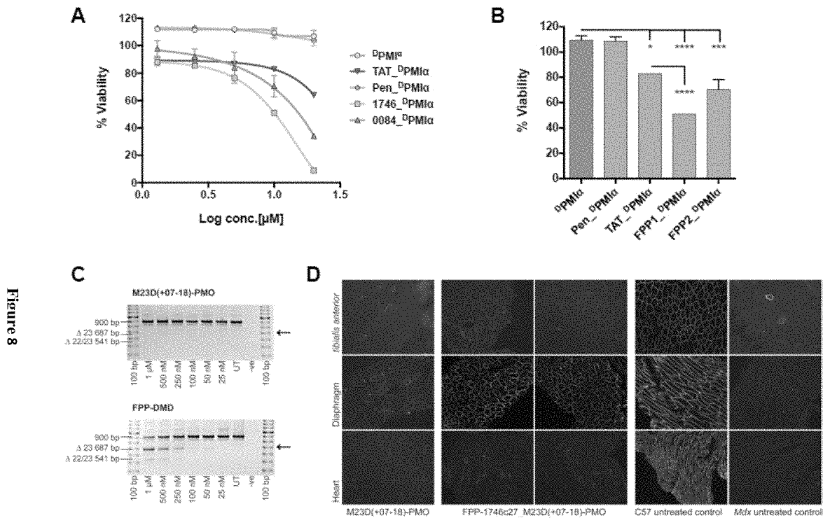

^{D}PMIα is a small peptide (SEQ ID NO:105), which when conjugated to cationic CPPs can internalize into cells, bind to MDM2 (acting as a dominant negative peptide) and lift p53 suppression causing indiscriminate cytotoxicity (Liu, supra). FPP_^{D}PMIα fusions were toxic to T47D cells, indicating they had been successfully delivered into the cell (FIG. 8A). FPP1_^{D}PMIα (IC 50 9.1 μM; SEQ ID NO:106) was. significantly more cytotoxic than ^{D}PMIα alone, and showed greater potency than TAT_^{D}PMIα (IC50 36.6 μM; SEQ ID NO:107). In addition, Penetratin fused to ^{D}PMIα did not cause any evidence of cell toxicity in T47D cells (FIG. 8B).

In another delivery assay a modified CPP was generated by linking FPP1.1 (SEQ ID NO:81) to a phosphorodiamidate-morpholino oligomer M23D(+7-18), which targets exon 23 of the Dystrophin gene that is mutated in in certain cases of Duchenne muscular dystrophy (DMD). Intracellular delivery of M23D(+7-18) induces exon skipping to produce a shorter, yet functional dystrophin protein. FPP1.1 successfully delivered M23D(+7-18) into murine H-2Kb-tsA58 myoblasts in vitro, with exon skipping detectable at the RNA level when cells were treated with as little as 50 nM of FPP1.1_M23D(+7-18) cargo (FIG. 8C).

In vivo delivery was assessed by treating C57BL/10ScSn^{mdx} mice with five intra-peritoneal injections over two weeks at 4 nmoles per dose, of either FPP1.1_M23D(+7-18) or M23D(+7-18) oligonucleotide alone. Two weeks after end of treatment, tissue cryosections from mice treated with FPP1.1_M23D(+7-18) showed an increase in dystrophin expression and improved muscle architecture in the diaphragm compared to untreated and M23D(+7-18) only-treated mice. Moderate improvement was also seen in the tibialis anterior (FIG. 8D).

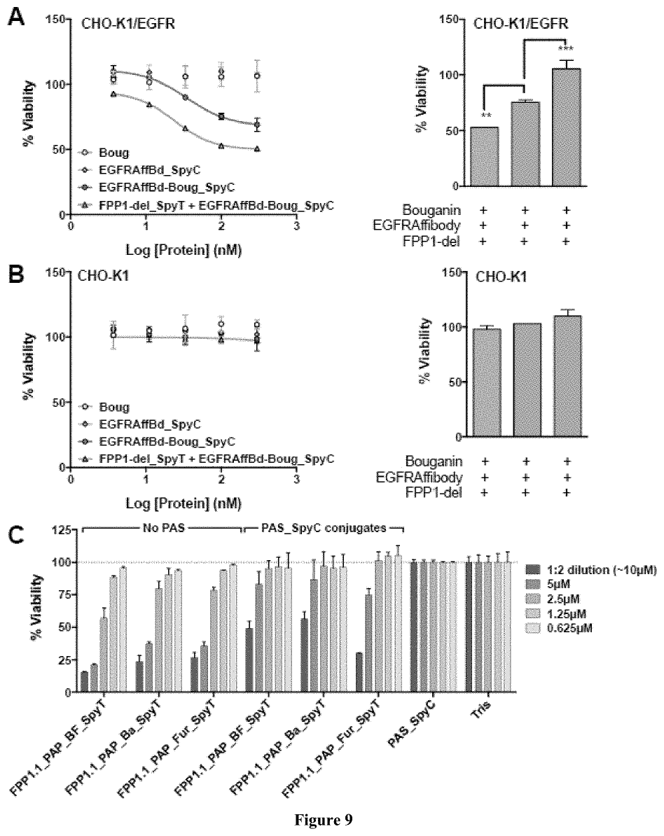

The potent cargo activities in diverse cell lines suggest that intracellular delivery by FPP1.1 is not cell-specific. To demonstrate the compatibility of a Phylomer™ FPP with cell targeting approaches, we generated a fusion protein (SEQ ID NO:109) linking Affibody_{EGFR-1907}, a well-characterized targeting domain (Friedman et al 2008, J Mol Biol, 376, 1388-1402) that binds hEGFR, a potent cytotoxic enzyme Bouganin (Hartog et al 2002, Eur J Biochem, 269, 1772-1779; Bolognesi et al 2000, Br J Haematol, 110, 351-361) and assessed its delivery, when conjugated with Phylomer™ FPPs in matched CHO-K1 cell lines (±hEGFR receptor). Importantly, the Phylomer™ FPP-delivered toxin was highly potent only in hEGFR-positive cells, showing that it retained Affibody-conferred cell-specificity (FIGS. 9A, 9B).

We also performed preliminary assessment on the versatility of our FPPs in the context of drug development by assessing the effect of a standard half-life extension on the potency of a Phylomer™ FPP_Cargo. We expressed SpyC as a recombinant fusion with PAS protein, a large hydrophobic protein often used for half-life extension of biologics (Schlapschy et al 2013, Protein Eng Des Sel, 26, 489-501). PAS_SpyC was conjugated to SpyTag-containing FPP1.1 PAP fusion peptide (SEQ ID NO:97) using three proteolytically cleavable linker variants and then applied to T47D cells. The cleavable linker motifs used were Cathepsin B FKFL cleavage motif (BF) (Chu et al 2012 J. Contr. Rel., 157:445-454), Cathepsin B Valine-Citrulline cleavage motif (Ba) (Liang et al 2012, J. Contr. Rel., 160:618-629) and Furin RKKR cleavage motif (Fur) (Thomas 2002, Nat. Rev. Mol. Cell Biol. 3:753-766). While there was some decrease in potency from the addition of the large PAS molecule, FPP-dependent PAP-induced cytotoxicity was still detected for all PAS conjugates (FIG. 9C). The Furin-cleavable conjugate showed the greatest potency, consistent with the hypothesis that while half-life improves from addition of PAS it is important to allow subsequent cleavage of such a large hydrophobic protein from the FPP-delivered cargo to improve the efficiency of uptake and endosomal escape. Together, these proof of concept studies show that potent non-specific Phylomer™ FPPs can be re-engineered to be cell specific if required, that they are amenable to standard half-life extension technologies, and are thus compatible with targeted, stabilized, recombinant therapeutics.

### Example 4: Characterizing Delivery and Uptake Mechanism

To assess potential mechanism and kinetics of uptake and endosomal release, we labelled FPP1.1 (SEQ ID NO:81) with the pH-sensitive fluorescent probe carboxynaphthofluorescein (NF), (Qian, supra) which is non-fluorescent at low pH values observed in the endosomes (pH<6).

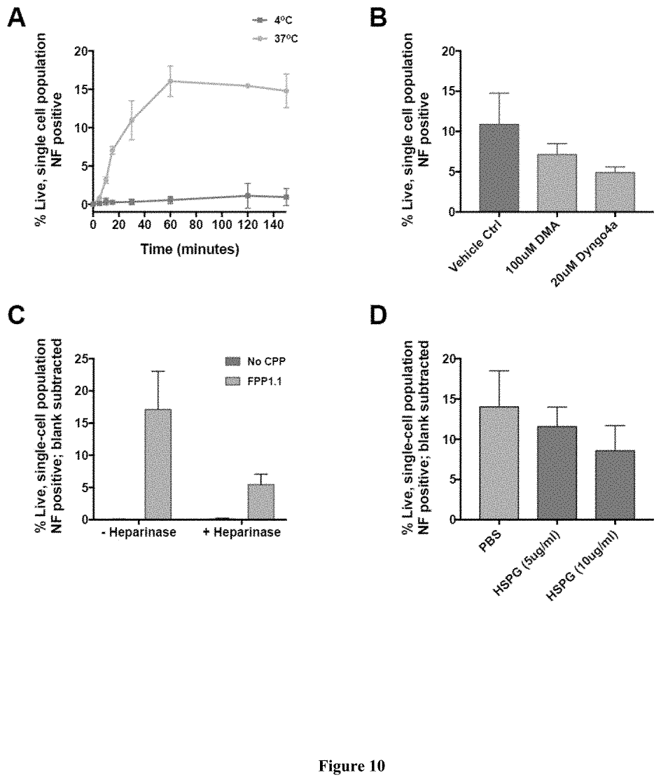

Endosomal release and uptake occurred rapidly and could reliably be detected as early as 10 mins post addition of FPP1.1 modified by linkage to NF (NF-FPP1.1), with fluorescence levels plateauing at around 60 mins (FIG. 10A). The time and temperature-dependent increase in NF-FPP1.1 signal suggests that peptide uptake could be through endocytotic or energy dependent pathways. Preliminary experiments using Dyngo 4a, an inhibitor of clathrin-mediated endocytosis, and DMA, an inhibitor of macropinocytosis, showed that pre-treating cells with endocytotic inhibitors resulted in a trend for decrease in NF-FPP1.1 uptake (FIG. 10B). We then assessed the potential of NF-FPP1.1 to bind to cell membrane heparin sulfate proteoglycans (HSPGs). Pre-treatment of cells with heparinase (III) showed reduced uptake of NF-FPP1.1 (FIG. 10C). Similarly, after pre-incubating the FPP with HPSG to compete with cell-membrane-displayed HPSGs for FPP-binding, we observed a dose-dependent decrease in NF-FPP1.1 uptake (FIG. 10D). These findings suggest that binding to cellular HSPG is required for maximal FPP1.1 internalization.

### Example 5: Therapeutic Relevant Protein Cargo Against an Intracellular Target

Finally, we assessed the ability of FPP1.1 to deliver a therapeutically-relevant cargo that directly acts on an intracellular target deemed to be “undruggable” by traditional biologic-therapeutics approaches. MYC is a prototypic example of an “undruggable target” whose deregulation is a hallmark of cancer due to its role as a master regulator of stem cell state, embryogenesis, tissue homeostasis, and aging. We used Omomyc (Soucek et al 1998, Oncogene, 17, 2463-2472; Soucek et al 2002, Cancer Res, 62, 3507-3510) a small structured dominant negative protein with known activity against cMYC, as our therapeutic cargo and recombinantly-expressed it as a direct fusion with FPP1.1.

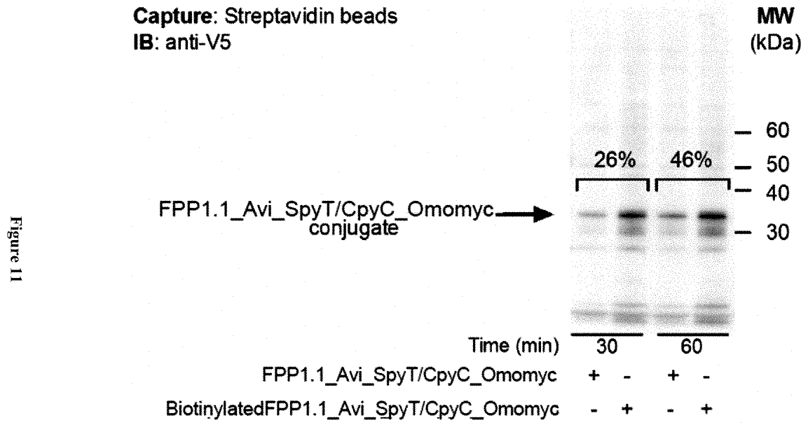

We first validated intracellular delivery of recombinant SpyC_Omomyc protein (SEQ ID NO:110) conjugated to an FPP1.1_Avitag_SpyTag fusion peptide (SEQ ID NO:111) in HEK-293_BirA, biotin ligase-expressing cells (FIG. 11) by detecting biotinylated conjugate by immunoblotting biotinylated proteins pulled-down from cellular lysate by streptavidin magnetic beads. Ex vivo biotinylation of the Avitag sequence contained in the SpyT peptide was successfully detected, and could occur only if the conjugate was delivered into the cytoplasm of the cells where active BirA enzyme is expressed. We then expressed FPP1.1_Omomyc recombinant protein as our biologic therapeutic.

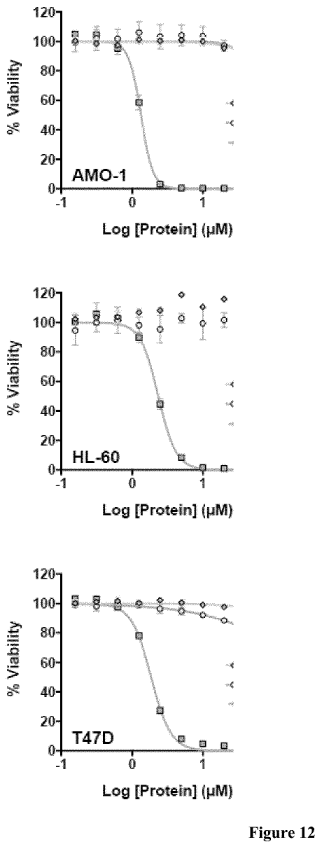

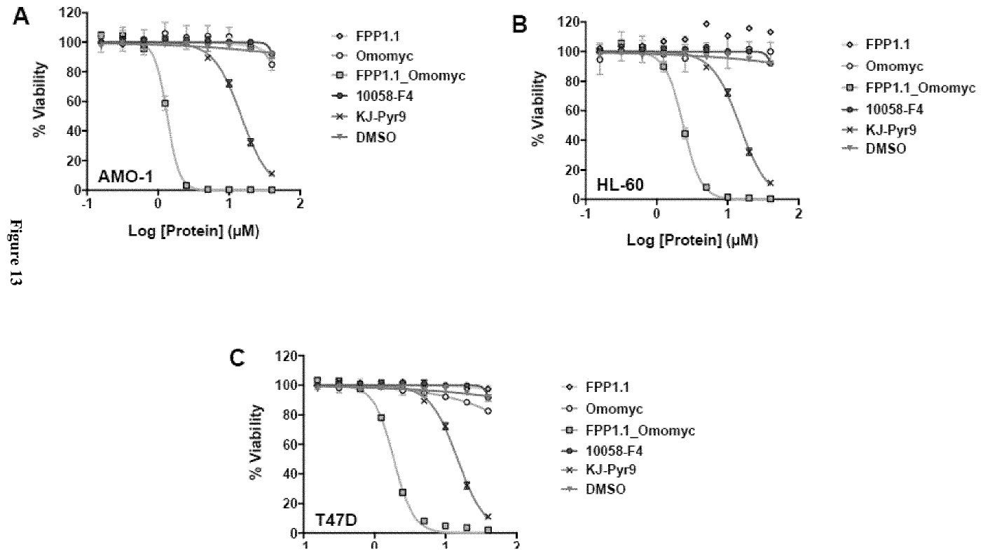

When Myc-dependent blood cancer cell lines, AMO-1 and HL-60, and the breast cancer cell line, T47D, were treated with recombinant FPP1_Omomyc, we observed a dose-dependent decrease in cell viability (FIGS. 12A-C). This intracellular therapeutic was particularly potent, with average IC50s of 1.28 μM (AMO-1), 1.88 μM (HL-60) and 1.67 μM (T47D). Complete cell death was observed at concentrations≥5 μM (AMO-1) or ≥10 μM (HL-60 and T47D), with some efficacy retained even at doses as low as 0.625-1.25 μM. Remarkably, the potency of FPP1.1_Omomyc was greater than that of small molecule MYC inhibitors, 10058-F4 and KJ-Pyr9 in three different cell lines (AMO-1, HL-60, and T47D; FIGS. 13A-C). In contrast, as expected, no effect on cell viability was seen for recombinant Omomyc alone (SEQ ID NO:99), with the exception of AMO-1 cells where a slight reduction in cell viability was seen at doses above 10 μM of recombinant Omomyc. Treatment with FPP1.1 alone resulted in no significant cytotoxicity.

In the above examples, we have demonstrated, identified and validated several Phylomer™-based CPPs as bona fide FPPs. FPP1 and several variants (e.g, FPP1.1) showed greater delivery activity than conventional CPPs, particularly at lower concentrations where uptake is less likely to be related to the phenomenon of non-specific flooding entry into cells (Verdurmen et al 2010, J of Controlled Release, 147, 171-179) When evaluating CPPs as therapeutic delivery agents the sequence needs to be versatile enough to accommodate various typical maturation modifications that may be required. Using clustering analysis of FPP1 to guide basic affinity maturation, we engineered peptide FPP1.1, which retains the strong potency of the parental sequence yet is amenable to synthesis. FPP1.1 is also potent as a dimer and as a retro-inverso sequence, a strategy often used to render peptides less susceptible to proteolytic cleavage (Fischer et al 2003, Curr Protein Pept. Sci., 4, 339-356). Finally, FPP1.1 was compatible with basic half-life extension and targeting technologies often employed to overcome the lack of specificity and quick clearance typically seen with traditional CPPs (Sarko et al 2010, Mol. Pharmaceutics, 7, 2224-2231) showing EGFR-dependent specificity when combined with a targeting Affibody and retaining a degree of potency after PASylation. Initial kinetics assessment of FPP1.1 established that uptake is rapid, is energy dependent and sensitive to inhibitors of endocytosis. This suggests that uptake occurs through clathrin-mediated endocytosis and is enhanced by heparin sulfate binding, consistent with its viral origin (Bomsel et al 2003, Nat Rev Mol Cell Biol, 4, 57-68) These mechanisms have been reported to function in a “piggyback” manner (Jones et al 2012, Journal of controlled release: official journal of the Controlled Release Society, 161, 582-591; Qian et al 2014, Biochemistry 53, 4034-4046) where the peptide is potentially internalized while bound to HSPG, facilitating endocytosis.

Here we deliberately chose a cargo-focused approach to validate our Phylomer FPPs and showed successful delivery of multiple larger and biologically relevant cargoes, evidence of which is rare in the CPP field (reviewed in Kauffman et al 2015, Trends Biochem Sci 40, 749-764). We recently reported a functional cytoplasmic delivery assay which showed striking differences between the potency of ten well-characterized canonical CPP (Milech, supra). Only TAT, R9 and Penetratin successfully delivered the protein cargo into the cytoplasm of cells. Of these, TAT-mediated delivery was the most successful at concentrations lower than 10 μM. In contrast, FPP1 retains strong potency and shows great versatility in delivering a variety of biological cargoes at concentrations down to single-digit micromolar to sub-micromolar-concentrations. This is highly desirable in the therapeutic context, as it avoids the need for dosing at high concentrations, which can induce translocation, toxicity, membrane disruption and increase the costs of manufacturing. As proof-of-concept for therapeutic application, we used a Phylomer™ FPP to deliver Omomyc, a well characterized protein inhibitor of cMYC with poor cellular penetration. Omomyc alone showed poor potency, causing only a slight reduction in cell viability at doses above 10 μM in one cell line. In sharp contrast, FPP1.1_Omomyc showed IC50s in the low single digit micromolar range (1.3-1.9 μM).

To our knowledge, these potencies are unprecedented for direct targeting of cMYC with a small molecule or protein-based biological therapy, and hence demonstrates the potential utility of Phylomer FPP-mediated delivery of a biologic therapeutic. We also have shown potent delivery of recombinant β-lactamase, detectable at sub-micromolar concentrations, as well as delivery of recombinant PAP and ^{D}PMIα peptide with greater potency compared to the conventional CPPs assessed alongside. Finally, when mice were treated with FPP-delivered morpholino oligos we observed a partial reversion of cell phenotype to normal morphology, providing strong evidence for the power of Phylomer FPPs to deliver high-potency therapeutics, including polynucleotides, in vivo.

In summary, we have identified a subset of Phylomer™-based CPPs that show functional cell penetration, endosomal escape and cytoplasmic uptake, which we refer to as “FPPs.” We have demonstrated that these FPPs are potent, versatile, and compatible with engineering solutions to further improve endosomal escape (Shin et al 2017, Nat Commun, 8, 15090. In addition, these FPPs are amenable to synthesis, and recombinant production where the FPP sequence encodes only naturally occurring (canonical) amino acids and thus compatible with cost-efficient scaled manufacturing. Further, these FPPs are generally not cytotoxic and importantly, are able to deliver into cells a wide range of biologic cargoes ranging from large proteins to small peptides and oligonucleotides, both in vitro and in vivo. We propose that the innate delivery efficiency of Phylomer™ FPPs addresses a key challenge for intracellular-targeted biologics by enabling more biologic drug payload to reach diverse disease targets within the cell.

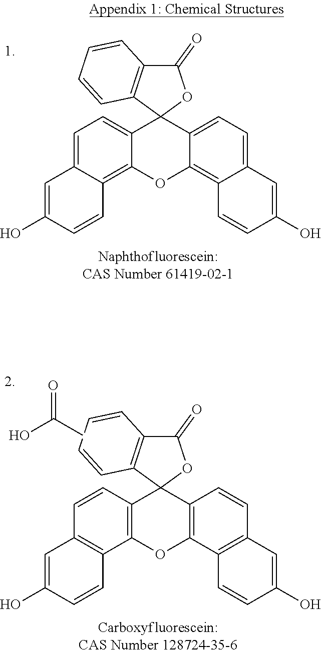

APPENDIX 2
Sequences and SEQ ID NOs:
(Note: amino acids designated in lower case refer to D-amino acids)

SEQ ID NO: 1
KTQE (X1-A)
SEQ ID NO: 2
RTQE (X1-B)
SEQ ID NO: 3
QPAKPRPKTQE (X3-A)
SEQ ID NO: 4
QPPKPKKPKTQE (X3-B)
SEQ ID NO: 5
QPPRPRRPRTQE (X3-C)
SEQ ID NO: 6
QTTKTKKTKTQE (X3-D)
SEQ ID NO: 7
QPAKKKPKTQE (X3-E)
SEQ ID NO: 8
QAPKQPPKPKKPKTQE (X3-F)
SEQ ID NO: 9
QPPKPKR (X5-A)
SEQ ID NO: 10
QTTKTKR (X5-B)
SEQ ID NO: 11
QPPKPK (X5-C)
SEQ ID NO: 12
QPPRPRR (X5-D)
SEQ ID NO: 13
RTRLQPPRPRPPPRQKKQAPKQPPKPKKPKTQEKKKKQPAKPKPGKRQRMALK
LEADRLFDVKNEDGDVIDHTVKEGTMDDIKISASGQRTPPRPRPPPRQKKQAPK
QPPKPKKPKTQEKKKKQPAKPKPGKRQRMALKLEADRLFDVKNEDGDVIDHT
VKEGTMDDIKISASGQ (SVC1)
SEQ ID NO: 14
PKLQERRKKQPPKPKKPKTQERKKKQPAKPKPGKRQR
MALKLEADRLFDVKNEDGDVIGHALAMEGKVMKPLHVKGTIDHPVLSKLNA
(SVC2)
SEQ ID NO: 15
LKPKKPKTQEKKKKQPAKPKPGKRQRMALKLEADRLFDVKNEDGDVIGHALA
MEGEVMKPLHVKGTIDHPVLSKLKFTKSSNA (SVC3)
SEQ ID NO: 16
QAPKQPPKPKKPKTQEKKKKQPAKPKPGKRQRMALKLEADRLFDVKNEDGD
VIGHALAMEGKVMKPLHVKGTIDHSS (SVC4)
SEQ ID NO: 17
QATQEKKKKQPAKPKKPKTQEKKKKQPAKPKPGKRQRMALKLEADRLFDVK
NEDGDVIGHALAMEGKVMKPLHVNG (SVC5)
SEQ ID NO: 18
PKPQEKKKKQPAKPKKPKTQEKKKKQPAKPKPGKRQRMALKLEADRLFDVK
NEDGDVICTQATRGQQRQIGR (SVC6)
SEQ ID NO: 19
PKTQEKKKKQPAKPKPGKRQRMALKLEADRLFDVKNEDGDVIGHALAMEGK
VMKPLHVKGTIDHPVLSKLIC (SVC7)
SEQ ID NO: 20
HKKKAPKQPPKPKKPKTQEKKKKQPAKPKPGKRQRMALKLEADRLFDVKNE
DGDVIGHALAMEGKVMKKQ (SVC8)
SEQ ID NO: 21
LPPPRQKKQAPKQPPKPKKPKTQEKKKKQPAKPKPGKRQRMALKLEADRLSD
VKNEDGDVIGHALARFV (SVC9)
SEQ ID NO: 22
QKSQEKKKKQPAKPKKPKTQEKKKKQPAKPKPGKRQRMALKLEADRLFDVK
NEDGDVIGHALAMEVKA (SVC10)
SEQ ID NO: 23
PPRQKKQAPKQPPKPKKPKTQEKKRKQPAKPKPGKRQRMALKLEADRLFDVK
NEDGDVIGHALARKA (SVC11)
SEQ ID NO: 24
QTQEKKKKQPAKPKKPKTQEKKKKQPAKPKPGKRQRMALKLEADRLFDVKN
EDGDVIGHALAMEAK (SVC12)
SEQ ID NO: 25
QKKQPPKQPPKPKKPKTQEKKKKQPAKPKPGNRQRMALKLEADRLFDVKNED
GDVIGHALAMEGEA (SVC13)
SEQ ID NO: 26
PKTQEKKKKQPAKPKPGKRQRMALKLEADRLFDVKNEDGDVIGQAPKQPPKP
KKPKTQEKKKK (SVC14)
SEQ ID NO: 27
QATKQPPKPKKPKTQEKKKKQPAKPKPGKRQRMALKLEADRLFDVKNEDGD
VIGHALAMKT (SVC15)
SEQ ID NO: 28
QAPKQPPKPKKPKTQEKKKKQPAKPKPGKRQRMALKLEADRLFDVKNEDGD
VIGHALATKG (SVC16)
SEQ ID NO: 29
LKTQEKKKKQPAKPKKPKTQEKKKKQAPKQPPKPKKPKTQEKKKKQPAKPKK
PKTQEKKKA (SVC17)
SEQ ID NO: 30
PKPKKPKTQEKKKKQPAKPKPGKRQRMALKLEADRLFDVKNEDGDVIGHALA
MEGKVMKP (SVC18)
SEQ ID NO: 31
PKQPPKPKKPKTQEKKKKQPAKPKPGKRQRMALKLEADRLFDVKNEDGDVIG
HALDMKA (SVC19)
SEQ ID NO: 32
PKQPPKPKKPKTQEKKKKQPAKPKKPKTQEKKKKQPAKPRPKTQEKKKKQPA
(SVC20)
SEQ ID NO: 33
QTAKQPPKPKKPKTQEKKKKQPAKPKPGKRQRMALKLEADRLFDVKNEDGG
A (SVC21)
SEQ ID NO: 34
PKTQEKKKKQAPKQPPKPKKPKTQEKKKKQPAKPKPGKRQRMALKLEADRV
C (SVC22)
SEQ ID NO: 35
QVKEKKQPAKPKKPKTQEKKKKQPAKPRPGKRQRMALKLEADRLFDVKDKG
(SVC23)
SEQ ID NO: 36
PKQPPKPKKPKTQEKKKKQPAKPKPGKRQRMALKLEADRLFDVKMKMAT
(SVC24)
SEQ ID NO: 37
PNPPEKKKKQPAKPKKPKTQEKKKKQPAKPKPGKRQRMALKLEEGA (SVC25)
SEQ ID NO: 38
QAPKQPPKPKKPKTQEKKKKQPPKPKKPKTQEKKKKQA (SVC26)
SEQ ID NO: 39
PLKPKKPKTQEKKKKQPPKPKKPKTQEKKKKQPPKPKR (FPP1)
SEQ ID NO: 40
PNAQEKKKKQPPKPKKPKTQEKKKKQPPKPKKPKTQ (SVC28)
SEQ ID NO: 41
PPKPKKPKTQEKKKKQPAKPKPGKRQRMALKLEAYA (SVC29)
SEQ ID NO: 42
Ac-KTQEKKKKQPPKPKKPKTQEKKKKQPPKPKRGASAHIVMVDAYKPTKG-
nh2 (CST1)
SEQ ID NO: 43
Ac-PKKPKTQEKKKKQPPKGASAHIVMVDAYKPTKG-nh2 (CST2)
SEQ ID NO: 44
Ac-PLKPKKPKTQEKKKKQPPKPKGASAHIVMVDAYKPTKG-nh2 (CST3)
SEQ ID NO: 45
Ac-PLKPKKPKTQEKKKKQPPKPKKPKTGASAHIVMVDAYKPTKG-nh2 (CST4)
SEQ ID NO: 46
Ac-PKTQEKKKKQPPKPKKPKTQEKKKKQPGASAHIVMVDAYKPTKG-nh2
(CST5)
SEQ ID NO: 47
Ac-KKQPPKPKKPKTQEKKKKQPPKPKRGASAHIVMVDAYKPTKG-nh2 (CST6)
SEQ ID NO: 48
Ac-PRQKKQAPKQPPKPKKPKTQEKKKKQPGASAHIVMVDAYKPTKG-nh2
(CST7)
SEQ ID NO: 49
Ac-TKTQEKKKKQTTKTKKTKTQEKKKKQTGASAHIVMVDAYKPTKG-nh2
(CST8)
SEQ ID NO: 50
Ac-PKTQEAAAAQPPKPKKPKTQEAAAAQPGASAHIVMVDAYKPTKG-nh2
(CST9)
SEQ ID NO: 51
Ac-
PLKPKKPKTQEKKKKQPPKPKKPKTQEKKKKQPPKPKGASAHIVMVDAYKPT
KG-nh2 (CST10)
SEQ ID NO: 52
Ac-KPKKPKTQEKKKKQPPKPKKPKTQEKKKKQPPKPKRGASAHIVMVDAYK
PTKG-nh2 (CST11)
SEQ ID NO: 53
Ac-KKPKTQEKKKKQPPKPKKPKTQEKKKKQPPKPKRGASAHIVMVDAYK
PTKG-nh2 (CST12)
SEQ ID NO: 54
Ac-KKQPPKPKKPKTQEKKKKQPPKPKKPKTQEKKGASAHIVMVDAYKPTKG-
nh2 (CST13)
SEQ ID NO: 55
Ac-TLKTKKTKTQEKKKKQTTKTKKTKTQEKKKKQTTKTKRGASAHIVMVDA
YKPTKG-nh2B (CST14)
SEQ ID NO: 56
Ac-PLRPRRPRTQERRRRQPPRPRRPRTQERRRRQPPRPRRGASAHIVMVDAYK
PTKG-nh2 (CST15)
SEQ ID NO: 57
Ac-KTQEKKKKQTTKTKKTKTQEKKKKQTTKTKRGASAHIVMVDAYKPTKG-
nh2 (CST16)
SEQ ID NO: 58
Ac-QEKKKKQPPKPKKPKTQEKKKKQPPKPKRGASAHIVMVDAYKPTKG-nh2
(CST17)
SEQ ID NO: 59
Ac-KKKKQPPKPKKPKTQEKKKKQPPKPKRGASAHIVMVDAYKPTKG-nh2
(CST18)
SEQ ID NO: 60
Ac-PKTQEKKKKQPPKPKKPKTQEKKKKGASAHIVMVDAYKPTKG-nh2
(CST19-FPP1-SAR19-SpyTag)
SEQ ID NO: 61
KLAKLAKKLAKLAK (PAP1)
SEQ ID NO: 62
MGHHHHHHGATLEVLFQGPGGSDSATHIKFSKRDEDGKELAGATMELRDSSG
KTISTWISDGQVKDFYLYPGKYTFVETAAPDGYEVATAITFTVNEQGQVTVNG
KATKGGSGTGATSGKLAKLAKKLAKLAK (SC-PAP)
SEQ ID NO: 63
KLAKLAKKLAKLAKKLAK (PAP2)
SEQ ID NO: 64
KTQEKKKKQPPKPKKPKTQEKKKKQPPKPKR (delFPP1)
SEQ ID NO: 65
PKKPKTQEKKKKQPPK (SAR1)
SEQ ID NO: 66
PLKPKKPKTQEKKKKQPPKPK (SAR2)
SEQ ID NO: 67
PLKPKKPKTQEKKKKQPPKPKKPKT (SAR3)
SEQ ID NO: 68
PKTQEKKKKQPPKPKKPKTQEKKKKQP (SAR4)
SEQ ID NO: 69
KKQPPKPKKPKTQEKKKKQPPKPKR (SAR5)
SEQ ID NO: 70
PRQKKQAPKQPPKPKKPKTQEKKKKQP (SAR6)
SEQ ID NO: 71
TKTQEKKKKQTTKTKKTKTQEKKKKQT (SAR7)
SEQ ID NO: 72
PKTQEAAAAQPPKPKKPKTQEAAAAQP (SAR9)
SEQ ID NO: 73
PLKPKKPKTQEKKKKQPPKPKKPKTQEKKKKQPPKPK
(SAR12)
SEQ ID NO: 74
KPKKPKTQEKKKKQPPKPKKPKTQEKKKKQPPKPKR
(SAR13)
SEQ ID NO: 75
KKPKTQEKKKKQPPKPKKPKTQEKKKKQPPKPKR
(SAR14)
SEQ ID NO: 76
KKQPPKPKKPKTQEKKKKQPPKPKKPKTQEKK (SAR15)
SEQ ID NO: 77
TLKTKKTKTQEKKKKQTTKTKKTKTQEKKKKQTTKTKR
(SAR16_PT)
SEQ ID NO: 78
PLRPRRPRTQERRRRQPPRPRRPRTQERRRRQPPRPRR
(SAR17)
SEQ ID NO: 79
KTQEKKKKQTTKTKKTKTQEKKKKQTTKTKR (FPP1-P_T)
SEQ ID NO: 80
QEKKKKQPPKPKKPKTQEKKKKQPPKPKR (FPP1-KT)
SEQ ID NO: 81
KKKKQPPKPKKPKTQEKKKKQPPKPKR (FPP1.1)
SEQ ID NO: 82
PLKPKKPKTQEKKKKQPPKPKKPKTQEKKKKQPPKPKRPLKPKKPKTQEKKKK
QPPKPKKPKTQEKKKKQPPKPKRPLKPKKPKTQEKKKKQPPKPKKPKTQEKKK
KQPPKPKR (SVC30-FPP1-3X)
SEQ ID NO: 83
MGHHHHHHGATLEVLFQGPGGSDSATHIKFSKRDEDGKELAGATMELRDSSG
KTISTWISDGQVKDFYLYPGKYTFVETAAPDGYEVATAITFTVNEQGQVTVNG
KATKGGSGTGATSG (SCP)
SEQ ID NO: 84
GASAHIVMVDAYKPTKG (STP)
SEQ ID NO: 85
LEQRAEELARENEELLEKEAEELEQENAELEGECQGLEARNRELKERAESV
(DNATF5)
SEQ ID NO: 86
YREQIKRVKDSDDVP (DNras1)
SEQ ID NO: 87
TIEDSYRKQVVID (DNras2)
SEQ ID NO: 88 (Formula I)
X¹-X²-X³-X⁴⁻X⁵ (Formula I), wherein:
X¹ is an optional amino acid sequence selected from the group consisting of:
QE; KTQE (SEQ ID NO: 1); and RTQE (SEQ ID NO: 2);
X² is any combination of 3 to 8 lysine and/or arginine residues;
X³ is an amino acid sequence selected from the group consisting of:
QPAKPRPKTQE (SEQ ID NO: 3), QPPKPKKPKTQE (SEQ ID NO: 4),
QPPRPRRPRTQE (SEQ ID NO: 5), QTTKTKKTKTQE (SEQ ID NO: 6),
QPAKKKPKTQE (SEQ ID NO: 7), and QAPKQPPKPKKPKTQE (SEQ ID NO: 8)
X⁴ is any combination of 3 to 8 arginine and/or lysine residues; and
X⁵ is an amino acid sequence selected from the group consisting of QPPKPKR
(SEQ ID NO: 9); QTTKTKR (SEQ ID NO: 10); QPPKPK (SEQ ID NO: 11); and
QPPRPRR (SEQ ID NO: 12).
SEQ ID NO: 89
PKTQEKKKKQPPKPKKPKTQEKKKK (FPP1-SAR19)
SEQ ID NO: 90
MGHHHHHHGATLEVLFQGPGGSGSDSATHIKFSKRDEDGKELAGATMELRDS
SGKTISTWISDGQVKDFYLYPGKYTFVETAAPDGYEVATAITFTVNEQGQVTV
NGKATKGSHPETLVKVKDAEDQLGARVGYIELDLNSGKILESFRPEERFPMMS
TFKVLLCGAVLSRIDAGQEQLGRRIHYSQNDLVEYSPVTEKHLTDGMTVRELC
SAAITMSDNTAANLLLTTIGGPKELTAFLHNMGDHVTRLDRWEPELNEAIPND
ERDTTMPAAMATTLRKLLTGELLTLASRQQLIDWMEADKVAGPLLRSALPAG
WFIADKSGAGERGSRGIIAALGPDGKPSRIVVIYTTGSQATMDERNRQIAEIGAS
LIKHWQLGSASGTTGATSGEF (SpyCatcher-β-lactamase fusion protein)
SEQ ID NO: 91
MGHHHHHHGATLEVLFQGPGGSGSDSATHIKFSKRDEDGKELAGATMELRDS
SGKTISTWISDGQVKDFYLYPGKYTFVETAAPDGYEVATAITFTVNEQGQVTV
NGKATKGSHPETLVKVKDAEDQLGARVGYIELDLNSGKILESFRPEERFPMMS
TFKVLLCGAVLSRIDAGQEQLGRRIHYSQNDLVEYSPVTEKHLTDGMTVRELC
SAAITMSDNTAANLLLTTIGGPKELTAFLHNMGDHVTRLDRWEPELNEAIPND
ERDTTMPAAMATTLRKLLTGELLTLASRQQLIDWMEADKVAGPLLRSALPAG
WFIADKSGAGERGSRGIIAALGPDGKPSRIVVIYTTGSQATMDERNRQIAEIGAS
LIKHWQLGSASGTTGATSGEFKKKKQPPKPKKPKTQEKKKKQPPKPKR
(SpyC-BLA-FPP1.1)
SEQ ID NO: 92
RQIKIWFQNRRMKWKK (PenCPP)
SEQ ID NO: 93
GRKKRRQRRR (TATCPP)
SEQ ID NO: 94
GGTAGSTGGAHIVMVDAYKPTKG (LL1-ST)
SEQ ID NO: 95
GRKKRRQRRRGASAHIVMVDAYKPTKG (TAT-ST1)
SEQ ID NO: 96
RQIKIWFQNRRMKWKKGASAHIVMVDAYKPTKG (TAT-ST2)
SEQ ID NO: 97
KKKKQPPKPKKPKTQEKKKKQPPKPKRGASAHIVMVDAYKPTKG (FPP1.1-ST)
SEQ ID NO: 98
KKKKQPPKPKKPKTQEKKKKQPPKPKRHHHHHHSDTEENVKRRTHNVLERQR
RNELKRSFFALRDQIPELENNEKAPKVVILKKATAYILSVQAETQKLISEIDLLR
KQNEQLKHKLEQLRNSCA (FPP1.1-Omomyc)
SEQ ID NO: 99
GPGGSGTGATSDTEENVKRRTHNVLERQRRNELKRSFFALRDQIPELENNEKA
PKVVILKKATAYILSVQAETQKLISEIDLLRKQNEQLKHKLEQLRNSCA
(Omomyc)
SEQ ID NO: 100
MASKKKKQPPKPKKPKTQEKKKKQPPKPKRASSGSHHHHHHGATLEVLFQGP
GGSDSATHIKFSKRDEDGKELAGATMELRDSSGKTISTWISDGQVKDFYLYPG
KYTFVETAAPDGYEVATAITFTVNEQGQVTVNGKATKGTSGAGKPIPNPLLGL
DST (FPP1.1 SpyC)
SEQ ID NO: 101
KKKKQPPKPKKPKTQEKKKKQPPKPKRGASAHIVMVDAYKPTKGASKKKKQP
PKPKKPKTQEKKKKQPPKPKR (FPP1.1-dimer-ST)
SEQ ID NO: 102
PLKPKKPKTQEKKKKQPPKPKKPKTQEKKKKQPPKPKRGASAHIVMVDAYKP
TKG (FPP1-ST)
SEQ ID NO: 103
RKQKSLQTKLAENPPVPRKKRQSRPRWKQWLQKGASAHIVMVDAYKPTKG
(FPP2-ST)
SEQ ID NO: 104
PPHPRPLPAPAQSRKKQKGRAGRGHEKTGASVLRGPQKPHPLPAQLRGASAHI
VMVDAYKPTKG (FPP3-ST)
SEQ ID NO: 105
tnwyanlekllr (^{D}PMII peptide-Note: lower case letters denote D-form amino acids)
SEQ ID NO: 106
PLKPKKPKTQEKKKKQPPKPKKPKTQEKKKKQPPKPKRGAStnwyanlekllr
(FPP1_^{D}PMII peptide-Note: lower case letters denote D-form amino acids)
SEQ ID NO: 107
GRKKRRQRRRGAStnwyanlekllr (TAT_^{D}PMII peptide-Note: lower case letters
denote D-form amino acids)
SEQ ID NO: 108
RKQKSLQTKLAENPPVPRKKRQSRPRWKQWLQKGAStnwyanlekllr
(FPP3_^{D}PMII peptide-Note: lower case letters denote D-form amino acids)
SEQ ID NO: 109
MGHHHHHHGATLEVLFQGPGGSGSVDNKFNKEMWAAWEEIRNLPNLNGWQ
MTAFIASLVDDPSQSANLLAEAKKLNDAQAPKGTGSGTGSATSGSLAGSGATA
GTGSGYNTVSFNLGEAYEYPTFIQDLRNELAKGTPVCQLPVTLQTIADDKRFVL
VDITTTSKKTVKVAIDVTDVYVVGYQDKWDGKDRAVFLDKVPTVATSKLFPG
VTNRVTLTFDGSYQKLVNAAKVDRKDLELGVYKLEFSIEAIHGKTINGQEIAKF
FLIVIQMVSEAARFKYIETEVVDRGLYGSFKPNFKVLNLENNWGDISDAIHKSS
PQCTTINPALQLISPSNDPWVVNKVSQISPDMGILKFKSSKGSGATAGSAATGG
ATGGSDSATHIKFSKRDEDGKELAGATMELRDSSGKTISTWISDGQVKDFYLY
PGKYTFVETAAPDGYEVATAITFTVNEQGQVTVNGKATKGGAGSWSHPQFEK
G (EGFRAffBd-Boug_SpyC)
SEQ ID NO: 110
MASHHHHHHGATLEVLFQGPGGSDSATHIKFSKRDEDGKELAGATMELRDSS
GKTISTWISDGQVKDFYLYPGKYTFVETAAPDGYEVATAITFTVNEQGQVTVN
GKATKGGSGTGATSGSDTEENVKRRTHNVLERQRRNELKRSFFALRDQIPELE
NNEKAPKVVILKKATAYILSVQAETQKLISEIDLLRKQNEQLKHKLEQLRNSCA
TSGAGKPIPNPLLGLDST (SpyC_Omomyc)
SEQ ID NO: 111
KKKKQPPKPKKPKTQEKKKKQPPKPKRGASGLNDIFEAQKIEWHEGASAHIVM
VDAYKPTKGASGKPIPNPLLGLDST (FPP1.1_Avi_SpyT)
SEQ ID NO: 112 (BLA)
HPETLVKVKDAEDQLGARVGYIELDLNSGKILESFRPEERFPMMSTFKVLLCGA
VLSRIDAGQEQLGRRIHYSQNDLVEYSPVTEKHLTDGMTVRELCSAAITMSDN
TAANLLLTTIGGPKELTAFLHNMGDHVTRLDRWEPELNEAIPNDERDTTMPAA
MATTLRKLLTGELLTLASRQQLIDWMEADKVAGPLLRSALPAGWFIADKSGA
GERGSRGIIAALGPDGKPSRIVVIYTTGSQATMDERNRQIAEIGASLIKHW
(BLA)
SEQ ID NO: 113
PKKKKQPPKPKKPKTQEKKKKQPPKPKR (SAR20)
SEQ ID NO: 114
QPPKPKRKKKKQPPKPKKPKTQEKKKK (SAR21)
SEQ ID NO: 115
RKPKPPQKKKKEQTKPKKPKPPQKKKK (SAR22)
SEQ ID NO: 116
kkkkQPPKPKKPKTQEkkkkQPPKPKR (SAR23)
(lower case ″k″ indicates D-lysine)
SEQ ID NO: 117
kkkkQPPKPKKPKTQEKKKKQPPKPKR (SAR24)
SEQ ID NO: 118
kKkKQPPkPKkPKTQEkKkKQPPkPKR (SAR25)
SEQ ID NO: 119
KRRKQPPKPKKPKTQEKRRKQPPKPKR (SAR26)
SEQ ID NO: 120
RKKRQPPKPKKPKTQERKKRQPPKPKR (SAR27)
SEQ ID NO: 121
KPPKQPPKPKKPKTQEKPPKQPPKPKR (SAR28)
SEQ ID NO: 122
PKKPQPPKPKKPKTQEPKKPQPPKPKR (SAR29)
SEQ ID NO: 123
KKKKEPPKPKKPKTEEKKKKEPPKPKR (SAR30)
SEQ ID NO: 124
PEEEEQPPKPKKPKTQEEEEEQPPKPK (SAR31)
SEQ ID NO: 125
KKKKQPPEPEEPETQEKKKKQPPEPER (SAR32)
SEQ ID NO: 126
KKKKQPPAPAAPATQEKKKKQPPAPAR (SAR33)
SEQ ID NO: 127
KKKKQPPQPQQPQTQEKKKKQPPQPQR (SAR34)
SEQ ID NO: 128
KKKKQPPTPTTPTTQEKKKKQPPTPTR (SAR35)
SEQ ID NO: 129
KKKKQTTKTKKTKTQEKKKKQTTKTKR (SAR36)
SEQ ID NO: 130
KKKKQAAKAKKAKTQEKKKKQAAKAKR (SAR37)
SEQ ID NO: 131
KKKKQPPKPKKPKTQEQPPKPKKPKTQEKKKKQPPKPKR (SAR38)
SEQ ID NO: 132
KKKKQTTKTKKTKTQEQTTKTKKTKTQEKKKKQTTKTKR (SAR39)
SEQ ID NO: 133
KKKKQPPKPKKPKTKKKKQPPKPKKPKTKKKKQPPKPKR (SAR40)
SEQ ID NO: 134
KKKKQPPKPKKKKTQEKKKKQPPKPKR (SAR41)
SEQ ID NO: 135
KKKKQPPKKKKPKTQEKKKKQPPKPKR (SAR42)
SEQ ID NO: 136
KKKKQPPKKKKKKTQEKKKKQPPKPKR (SAR43)
SEQ ID NO: 137
KKKKQPPKPKKPKTQEKKKKQPPKKKR (SAR44)
SEQ ID NO: 138
KKKQPPKPKKPKTQEKKKQPPKPKR (SAR45)
SEQ ID NO: 139
KKKKKQPPKPKKPKTQEKKKKKQPPKPKR (SAR46)
SEQ ID NO: 140
KKKKKKQPPKPKKPKTQEKKKKKKQPPKPKR (SAR47)
SEQ ID NO: 141
KKKKKKKQPPKPKKPKTQEKKKKKKKQPPKPKR (SAR48)
SEQ ID NO: 142
KKKKKKKKQPPKPKKPKTQEKKKKKKKKQPPKPKR (SAR49)
SEQ ID NO: 143
KKKKKKQPPKPKKPKTQEKKKKQPPKPKR (SAR50)
SEQ ID NO: 144
KKKKQPPKPKKPKTQEQPPKPKR (SAR51)
SEQ ID NO: 145
KKKKKKKKQPPKPKKPKTQEKKKKQPPKPKR (SAR52)
SEQ ID NO: 146
KKKKQPPKPKKPKTQEQPPKPKR (SAR53)
SEQ ID NO: 147
KKKKKPPKPKKPKTQEKKKKQPPKPKR (SAR54)
SEQ ID NO: 148
KKKKKKPKPKKPKTQEKKKKQPPKPKR (SAR55)
SEQ ID NO: 149
KKKKKKKPKPKKPKTQEKKKKQPPKPKR (SAR56)
SEQ ID NO: 150
KKKKKKKKPKKPKTQEKKKKQPPKPKR (SAR57)
SEQ ID NO: 151
KKKKKKKKPKKPKTQEKKKKKPPKPKR (SAR58)
SEQ ID NO: 152
KKKKKKKKPKKPKTQEKKKKKKPKPKR (SAR59)
SEQ ID NO: 153
KKKKKKKKPKKPKTQKKKKKKKPKPKR (SAR60)
SEQ ID NO: 154
KKKKKKKKPKKPKTQEPKR (SAR61)
SEQ ID NO: 155
KKKKKKKPKPKKPKTKKKKKKKKPKPKR (SAR62)
SEQ ID NO: 156
KKKKKKPKPKKPKTKKKKKKKKPKPKR (SAR63)
SEQ ID NO: 157
KKKKQPPKPKKPKTKKKKKKKKPKPKR (SAR64)
SEQ ID NO: 158
KKKKQPPKPKKPKTKKKKKKKKPKPKR (SAR65)
SEQ ID NO: 159
OrOrOrOrQPPKPKKPKTQEOrOrOrOrQPPKPKR (SAR66)
(Or = Ornithine)
SEQ ID NO: 160
OrOrOrOrQPPOrPOrOrPOrTQEOrOrOrOrQPPOrPOrR (SAR67)
SEQ ID NO: 161
CtCtCtCtQPPKPKKPKTQECtCtCtCtQPPKPKR (SAR68)
(Ct = Citrulline)
SEQ ID NO: 162
CtCtCtCtQPPCtPCtCtPCtTQECtCtCtCtQPPCtPCtR (SAR69)
SEQ ID NO: 163
DbDbDbDbQPPKPKKPKTQEDbDbDbDbQPPKPKR (SAR70)
(Db = Diaminobutyric acid)
SEQ ID NO: 164
DbDbDbDbQPPDbPDbDbPDbTQEDbDbDbDbQPPDbPDbR (SAR71)
SEQ ID NO: 165
DpDpDpDpQPPKPKKPKTQEDpDpDpDpQPPKPKR (SAR72)
(Dp = Diaminopropionic acid)
SEQ ID NO: 166
DpDpDpDpQPPDpPDpDpPDpTQEDpDpDpDpQPPDpPDpR (SAR73)
SEQ ID NO: 167
KKKKQppKpKKpKTQEKKKKQppKpKR (SAR74)
(p = D-proline)
SEQ ID NO: 168
PKKKKQPPKPKKPKTQEKKKKQPPKPKRGASAHIVMVDAYKPTKG
(SAR20-ST)
SEQ ID NO: 169
QPPKPKRKKKKQPPKPKKPKTQEKKKKGASAHIVMVDAYKPTKG
(SAR21-ST)
SEQ ID NO: 170
RKPKPPQKKKKEQTKPKKPKPPQKKKKGASAHIVMVDAYKPTKG
(SAR22-ST)
SEQ ID NO: 171
kkkkQPPKPKKPKTQEkkkkQPPKPKRGASAHIVMVDAYKPTKG
(SAR23-ST)
SEQ ID NO: 172
kkkkQPPKPKKPKTQEKKKKQPPKPKRGASAHIVMVDAYKPTKG
(SAR24-ST)
SEQ ID NO: 173
kKkKQPPkPKkPKTQEkKkKQPPkPKRGASAHIVMVDAYKPTKG
(SAR25-ST)
SEQ ID NO: 174
KRRKQPPKPKKPKTQEKRRKQPPKPKRGASAHIVMVDAYKPTKG
(SAR26-ST)
SEQ ID NO: 175
RKKRQPPKPKKPKTQERKKRQPPKPKRGASAHIVMVDAYKPTKG
(SAR27-ST)
SEQ ID NO: 176
KPPKQPPKPKKPKTQEKPPKQPPKPKRGASAHIVMVDAYKPTKG
(SAR28-ST)
SEQ ID NO: 177
PKKPQPPKPKKPKTQEPKKPQPPKPKRGASAHIVMVDAYKPTKG
(SAR29-ST)
SEQ ID NO: 178
KKKKEPPKPKKPKTEEKKKKEPPKPKRGASAHIVMVDAYKPTKG
(SAR30-ST)
SEQ ID NO: 179
PEEEEQPPKPKKPKTQEEEEEQPPKPKRGASAHIVMVDAYKPTKG
(SAR31-ST)
SEQ ID NO: 180
KKKKQPPEPEEPETQEKKKKQPPEPERGASAHIVMVDAYKPTKG
(SAR32-ST)
SEQ ID NO: 181
KKKKQPPAPAAPATQEKKKKQPPAPARGASAHIVMVDAYKPTKG
(SAR33-ST)
SEQ ID NO: 182
KKKKQPPQPQQPQTQEKKKKQPPQPQRGASAHIVMVDAYKPTKG
(SAR34-ST)
SEQ ID NO: 183
KKKKQPPTPTTPTTQEKKKKQPPTPTRGASAHIVMVDAYKPTKG
(SAR35-ST)
SEQ ID NO: 184
KKKKQTTKTKKTKTQEKKKKQTTKTKRGASAHIVMVDAYKPTKG
(SAR36-ST)
SEQ ID NO: 185
KKKKQAAKAKKAKTQEKKKKQAAKAKRGASAHIVMVDAYKPTKG
(SAR37-ST)
SEQ ID NO: 186
KKKKQPPKPKKPKTQEQPPKPKKPKTQEKKKKQPPKPKRGASAHIVMVDAYK
PTKG (SAR38-ST)
SEQ ID NO: 187
KKKKQTTKTKKTKTQEQTTKTKKTKTQEKKKKQTTKTKRGASAHIVMVDAY
KPTKG (SAR39-ST)
SEQ ID NO: 188
KKKKQPPKPKKPKTKKKKQPPKPKKPKTKKKKQPPKPKRGASAHIVMVDAYK
PTKG (SAR40-ST)
SEQ ID NO: 189
KKKKQPPKPKKKKTQEKKKKQPPKPKRGASAHIVMVDAYKPTKG (SAR41-ST)
SEQ ID NO: 190
KKKKQPPKKKKPKTQEKKKKQPPKPKRGASAHIVMVDAYKPTKG (SAR42-ST)
SEQ ID NO: 191
KKKKQPPTQEKKKKQPPKPKRGASAHIVMVDAYKPTKG (SAR43-ST)
SEQ ID NO: 192
KKKKQPPKPKKPKTQEKKKKQPPKKKRGASAHIVMVDAYKPTKG (SAR44-ST)
SEQ ID NO: 193
KKKQPPKPKKPKTQEKKKQPPKPKRGASAHIVMVDAYKPTKG (SAR45-ST)
SEQ ID NO: 194
KKKKKQPPKPKKPKTQEKKKKKQPPKPKRGASAHIVMVDAYKPTKG
(SAR46-ST)
SEQ ID NO: 195
KKKKKKQPPKPKKPKTQEKKKKKKQPPKPKRGASAHIVMVDAYKPTKG
(SAR47-ST)
SEQ ID NO: 196
KKKKKKKQPPKPKKPKTQEKKKKKKKQPPKPKRGASAHIVMVDAYKPTKG
(SAR48-ST)
SEQ ID NO: 197
KKKKKKKKQPPKPKKPKTQEKKKKKKKKQPPKPKRGASAHIVMVDAYKPTK
G (SAR49-ST)
SEQ ID NO: 198
KKKKKKQPPKPKKPKTQEKKKKQPPKPKRGASAHIVMVDAYKPTKG
(SAR50-ST)
SEQ ID NO: 199
KKKKQPPKPKKPKTQEQPPKPKRGASAHIVMVDAYKPTKG
(SAR51-ST)
SEQ ID NO: 200
KKKKKKKKQPPKPKKPKTQEKKKKQPPKPKRGASAHIVMVDAYKPTKG
(SAR52-ST)
SEQ ID NO: 201
KKKKQPPKPKKPKTQEQPPKPKRGASAHIVMVDAYKPTKG
(SAR53-ST)
SEQ ID NO: 202
KKKKKPPKPKKPKTQEKKKKQPPKPKRGASAHIVMVDAYKPTKG
(SAR54-ST)
SEQ ID NO: 203
KKKKKKPKPKKPKTQEKKKKQPPKPKRGASAHIVMVDAYKPTKG
(SAR55-ST)
SEQ ID NO: 204
KKKKKKKPKPKKPKTQEKKKKQPPKPKRGASAHIVMVDAYKPTKG
(SAR56-ST)
SEQ ID NO: 205
KKKKKKKKPKKPKTQEKKKKQPPKPKRGASAHIVMVDAYKPTKG
(SAR57-ST)
SEQ ID NO: 206
KKKKKKKKPKKPKTQEKKKKKPPKPKRGASAHIVMVDAYKPTKG
(SAR58-ST)
SEQ ID NO: 207
KKKKKKKKPKKPKTQEKKKKKKPKPKRGASAHIVMVDAYKPTKG
(SAR59-ST)
SEQ ID NO: 208
KKKKKKKKPKKPKTQKKKKKKKPKPKRGASAHIVMVDAYKPTKG
(SAR60-ST)
SEQ ID NO: 209
KKKKKKKKPKKPKTQEKKKKKKKKPKRGASAHIVMVDAYKPTKG
(SAR61-ST)
SEQ ID NO: 210
KKKKKKKPKPKKPKTKKKKKKKKPKPKRGASAHIVMVDAYKPTKG
(SAR62-ST)
SEQ ID NO: 211
KKKKKKPKPKKPKTKKKKKKKKPKPKRGASAHIVMVDAYKPTKG
(SAR63-ST)
SEQ ID NO: 212
KKKKKPPKPKKPKTKKKKKKKKPKPKRGASAHIVMVDAYKPTKG
(SAR64-ST)
SEQ ID NO: 213
KKKKQPPKPKKPKTKKKKKKKKPKPKRGASAHIVMVDAYKPTKG
(SAR65-ST)
SEQ ID NO: 214
OrnOrnOrnOrnQPPKPKKPKTQEOrnOrnOrnOrnQPPKPKRGASAHIVMVDAYKPT
KG (SAR66-ST)
SEQ ID NO: 215
OrOrOrOrQPPOrPOrOrPOrTQEOrOrOrOrQPPOrPOrRGASAHIVMVDAYKPTKG
(SAR67-ST)
SEQ ID NO: 216
CitCitCitCitQPPKPKKPKTQECitCitCitCitQPPKPKRGASAHIVMVDAYKPTKG
(SAR68-ST)
SEQ ID NO: 217
CitCitCitCitQPPCitPCitCitPCitTQECitCitCitCitQPPCitPCitRGASAHIVMVDAYKP
TKG (SAR69-ST)
SEQ ID NO: 218
DabDabDabDabQPPKPKKPKTQEDabDabDabDabQPPKPKRGASAHIVMVDAYKP
TKG (SAR70-ST)
SEQ ID NO: 219
DabDabDabDabQPPDabPDabDabPDabTQEDabDabDabDabQPPDabPDabRGASAHI
VMVDAYKPTKG (SAR71-ST)
SEQ ID NO: 220
DapDapDapDapQPPKPKKPKTQEDapDapDapDapQPPKPKRGASAHIVMVDAYKP
TKG (SAR72-ST)
SEQ ID NO: 221
DapDapDapDapQPPDapPDapDapPDapTQEDapDapDapDapQPPDapPDapRGASAHI
VMVDAYKPTKG (SAR73-ST)
SEQ ID NO: 222
KKKKQppKpKKpKTQEKKKKQppKpKRGASAHIVMVDAYKPTKG (SAR74-ST)
SEQ ID NO: 223
QPPKPKR (X1-C)
SEQ ID NO: 224
RKPKPPQ (X1-D)
SEQ ID NO: 225
QPPkPKkPKTQE (X3-G)
SEQ ID NO: 226
EPPKPKKPKTEE (X3-H)
SEQ ID NO: 227
QPPEPEEPETQE (X3-I)
SEQ ID NO: 228
QPPAPAAPATQE (X3-J)
SEQ ID NO: 229
QPPQPQQPQTQE (X3-K)
SEQ ID NO: 230
QPPTPTTPTTQE (X3-L)
SEQ ID NO: 231
QAAKAKKAKTQE (X3-M)
SEQ ID NO: 232
QPPKPKKPKTQEQPPKPKKPKTQE (X3-N)
SEQ ID NO: 233
QTTKTKKTKTQEQTTKTKKTKTQE (X3-O)
SEQ ID NO: 234
QPPKPKKPKTKKKKQPPKPKKPKT (X3-P)
SEQ ID NO: 235
QPPKPKKKKTQE (X3-Q)
SEQ ID NO: 236
QPPKKKKPKTQE (X3-R)
SEQ ID NO: 237
QPPKKKKKKTQE (X3-S)
SEQ ID NO: 238
PPKPKKPKTQE (X3-T)
SEQ ID NO: 239
PKPKKPKTQE (X3-U)
SEQ ID NO: 240
PKKPKTQE (X3-V)
SEQ ID NO: 241
PKKPKTQ (X3-W)
SEQ ID NO: 242
PKPKKPKT (X3-X)
SEQ ID NO: 243
PPKPKKPKT (X3-Y)
SEQ ID NO: 244
QPPOrnPOrnOrnPOrnTQE (X3-Z)
SEQ ID NO: 245
QPPCitPCitCitPCitTQE (X3-AA)
SEQ ID NO: 246
QPPDabPDabDabPDabTQE (X3-AB)
SEQ ID NO: 247
QPPDapPDapDapPDapTQE (X3-AC)
SEQ ID NO: 248
QppKpKKpKTQE (X3-AD)
SEQ ID NO: 249
QPPkPKR (X5-E)
SEQ ID NO: 250
QPPEPER (X5-F)
SEQ ID NO: 251
QPPAPAR (X5-G)
SEQ ID NO: 252
QPPQPQR (X5-H)
SEQ ID NO: 253
QPPTPTR (X5-I)
SEQ ID NO: 254
QTTKTKR (X5-J)
SEQ ID NO: 255
QAAKAKR (X5-K)
SEQ ID NO: 256
QPPKKKR (X5-L)
SEQ ID NO: 257
PPKPKR (X5-M)
SEQ ID NO: 258
PKPKR (X5-N)
SEQ ID NO: 259
QPPOrnPOrnR (X5-P)
SEQ ID NO: 260
QPPCitPCitR (X5-Q)
SEQ ID NO: 261
GGGGS (LKR1)
SEQ ID NO: 262
GGGGSGGGGS (LKR2)
SEQ ID NO: 263
GGTAGSTGG (LKR3)

It will be appreciated by persons skilled in the art that numerous variations and/or modifications may be made to the above-described examples, without departing from the broad general scope of the present disclosure. The present examples are, therefore, to be considered in all respects as illustrative and not restrictive.

The present application claims priority from AU 2017902976 filed 28 Jul. 2017, the entire contents of which are incorporated herein by reference.

All references cited herein, including patents, patent applications, papers, text books, and the like, and the references cited therein, to the extent that they are not already, are hereby incorporated herein by reference in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1-58. (canceled)

59. A non-naturally occurring cell-penetrating peptide (CPP) comprising an amino acid sequence corresponding to one of the following structures:
(a) X¹-X²-X³-X⁴-X⁵ (Formula I), wherein:
X¹ is an optional amino acid sequence selected from the group consisting of: QE; KTQE (SEQ ID NO:1); and RTQE (SEQ ID NO:2);
X² is any combination of 3 to 8 lysine and/or arginine residues;
X³ is an amino acid sequence selected from the group consisting of: QPAKPRPKTQE (SEQ ID NO:3), QPPKPKKPKTQE (SEQ ID NO:4), QPPRPRRPRTQE (SEQ ID NO:5), QTTKTKKTKTQE (SEQ ID NO:6), QPAKKKPKTQE (SEQ ID NO:7), and QAPKQPPKPKKPKTQE (SEQ ID NO:8)
X⁴ is any combination of 3 to 8 arginine and/or lysine residues; and
X⁵ is an optional amino acid sequence selected from the group consisting of QPPKPKR (SEQ ID NO:9); QTTKTKR (SEQ ID NO:10); QPPKPK (SEQ ID NO:11); and QPPRPRR (SEQ ID NO:12), wherein the amino acid sequence of the non-naturally occurring CPP does not consist of the amino acid corresponding to:
(SEQ ID NO: 88)
PLKPKKPKTQEKKKKQPPKPKKPKTQEKKKKQPPKPKR.
or
(b) X¹-X²-X³-X⁴-X⁵ (Formula II), wherein:
X¹ is an optional amino acid sequence selected from the group consisting of: P; QE; KTQE (SEQ ID NO:1); RTQE (SEQ ID NO:2); QPPKPKR (SEQ ID NO:223); and RKPKPPQ (SEQ ID NO:224);
X² is any combination of 3 to 8 lysine and/or arginine residues;
X³ is an amino acid sequence selected from the group consisting of SEQ ID NOs:3-8 and 225-248;
X⁴ is any combination of 3 to 8 arginine and/or lysine residues; and
X⁵ is an optional amino acid sequence selected from the group consisting of SEQ ID NOS:9-12, 249-260, and PKR, wherein the amino acid sequence of the non-naturally occurring CPP does not consist of the amino acid corresponding to:
(SEQ ID NO: 88)
PLKPKKPKTQEKKKKQPPKPKKPKTQEKKKKQPPKPKR.

60. The CPP of claim 59, wherein:
(a) X² or X⁴ consists of only arginine residues;
(b) X² consists of only arginine residues;
(c) X⁴ consists of only arginine residues;
(d) X² and X⁴ consist of only arginine residues;
(e) X² or X⁴ consists of only lysine residues;
(f) X² consists of only lysine residues;
(g) X⁴ consists of only lysine residues;
(h) X² and X⁴ consist of only lysine residues;
(i) X² or X⁴ consists of both arginine and lysine residues; or
(j) X² and X⁴ consist of both arginine and lysine residues.

61. The CPP of claim 59, wherein the length of the amino acid sequence of the CPP consists of:
(a) 25 to 100 residues;
(b) 30 to 70 residues;
(c) 40 to 60 residues; or
(d) 25 to 50 residues.

62. The CPP of claim 59, wherein the amino acid sequence of the CPP comprises an amino acid sequence selected from:
(a) the group consisting of SEQ ID NOs:39, 64, 67, 69, and 73-81;
(b) the group consisting of SEQ ID NOs:64 and 74-81; or
(c) the group consisting of SEQ ID NOs:113-167.

63. The CPP of claim 59, wherein the CPP comprises multiple copies of an amino acid sequence corresponding to Formula I or Formula II.

64. The CPP of claim 59, wherein the amino acid sequence of the CPP consists of Formula I or Formula II.

65. The CPP of claim 59, wherein the CPP is a modified CPP comprising a moiety other than a canonical amino acid.

66. The modified CPP of claim 65, wherein the moiety is selected from the group consisting of a detectable label, a non-canonical amino acid, a reactive group, a fatty acid, cholesterol, a bioactive carbohydrate, a lipid, a nanoparticle, a small molecule drug, and a polynucleotide.

67. The modified CPP of claim 65, wherein the moiety is non-covalently linked to the CPP.

68. The modified CPP of claim 65, wherein the moiety is covalently linked to the CPP.

69. A CPP, wherein the amino acid sequence of the CPP is the retro-inverso sequence of the amino acid sequence of the CPP of claim 59.

70. A CPP fusion protein comprising the amino acid sequence of the CPP of claim and a heterologous amino acid sequence.

71. The CPP fusion protein of claim 70, wherein the heterologous amino acid sequence comprises an amino acid sequence selected from the group consisting of a SpyTag peptide (SEQ ID NO:84), a Phylomer™, a reporter protein, a pro-apoptotic peptide, a targeting protein, a cytotoxic protein, a bioactive peptide, a dominant negative peptide, an enzyme, an antibody, and a SpyC peptide (SEQ ID NO:83), wherein the Phylomer is a peptide of about 8 to about 180 amino acids encoded by nucleic acid fragments obtainable from genome(s) of a microorganisms or from a eukaryotic species having a compact genome.

72. The CPP fusion protein of claim 70, wherein the fusion protein comprises a flexible linker linking the CPP and the heterologous amino acid sequence.

73. A CPP conjugate comprising the fusion protein of claim 70 and a SpyCatcher fusion protein comprising the amino acid sequence of SEQ ID NO:83 and a heterologous amino acid sequence, wherein the SpyCatcher fusion protein is covalently linked to the CPP fusion protein by an isopeptide bond to the SpyTag peptide.

74. The CPP conjugate of claim 73, wherein the heterologous amino acid sequence in the SpyCatcher fusion protein comprises an amino acid sequence selected from the group consisting of a Phylomer™, a reporter protein, a pro-apoptotic peptide, an enzyme, a targeting protein, a cytotoxic protein, a dominant negative peptide, and an antibody, wherein the Phylomer is a peptide of about 8 to about 180 amino acids encoded by nucleic acid fragments obtainable from genome(s) of a microorganisms or from a eukaryotic species having a compact genome.

75. A modified cell comprising the CPP of claim 59.

76. A method for delivering a CPP to a cell, the method comprising contacting the cell with the CPP of claim 59.

77. The method of claim 76, wherein the contacting is performed ex vivo.

78. The method of claim 76, wherein the contacting is performed in vivo.


SEQUENCE LISTING (263 sequences)

SEQ ID NO: 1 - protein, 4 aa, Artificial Sequence
  CPP partial amino acid sequence of Formula I (X1-A)
KTQE

SEQ ID NO: 2 - protein, 4 aa, Artificial Sequence
  CPP partial amino acid sequence of Formula I (X1-B)
RTQE

SEQ ID NO: 3 - protein, 11 aa, Artificial Sequence
  CPP partial amino acid sequence of Formula I (X3-A)
QPAKPRPKTQE

SEQ ID NO: 4 - protein, 12 aa, Artificial Sequence
  CPP partial amino acid sequence of Formula I (X3-B)
QPPKPKKPKTQE

SEQ ID NO: 5 - protein, 12 aa, Artificial Sequence
  CPP partial amino acid sequence of Formula I (X3-C)
QPPRPRRPRTQE

SEQ ID NO: 6 - protein, 12 aa, Artificial Sequence
  CPP partial amino acid sequence of Formula I (X3-D)
QTTKTKKTKTQE

SEQ ID NO: 7 - protein, 11 aa, Artificial Sequence
  CPP partial amino acid sequence of Formula I (X3-E)
QPAKKKPKTQE

SEQ ID NO: 8 - protein, 16 aa, Artificial Sequence
  CPP partial amino acid sequence of Formula I (X3-F)
QAPKQPPKPKKPKTQE

SEQ ID NO: 9 - protein, 7 aa, Artificial Sequence
  CPP partial amino acid sequence of Formula I (X4-A)
QPPKPKR

SEQ ID NO: 10 - protein, 7 aa, Artificial Sequence
  CPP partial amino acid sequence of Formula I (X4-B)
QTTKTKR

SEQ ID NO: 11 - protein, 6 aa, Artificial Sequence
  CPP partial amino acid sequence of Formula I (X4-C)
QPPKPK

SEQ ID NO: 12 - protein, 7 aa, Artificial Sequence
  CPP partial amino acid sequence of Formula I (X4-D)
QPPRPRR

SEQ ID NO: 13 - protein, 175 aa, Artificial Sequence
  Amino acid sequence of a Sindbis virus capsid-derived CPP (SVC1)
RTRLQPPRPRPPPRQKKQAPKQPPKPKKPKTQEKKKKQPAKPKPGKRQRMALKLEADRLFDVKNEDGDVIDHTVKEGTMDDIKISASGQRTPPRPRPPPRQKKQAPKQPPKPKKPKTQEKKKKQPAKPKPGKRQRMALKLEADRLFDVKNEDGDVIDHTVKEGTMDDIKISASGQ

SEQ ID NO: 14 - protein, 88 aa, Artificial Sequence
  Amino acid sequence of a Sindbis virus capsid-derived CPP (SVC2)
PKLQERRKKQPPKPKKPKTQERKKKQPAKPKPGKRQRMALKLEADRLFDVKNEDGDVIGHALAMEGKVMKPLHVKGTIDHPVLSKLNA

SEQ ID NO: 15 - protein, 88 aa, Artificial Sequence; identical to SEQ ID NO: 14
  Amino acid sequence of a Sindbis virus capsid-derived CPP (SVC3)

SEQ ID NO: 16 - protein, 77 aa, Artificial Sequence
  Amino acid sequence of a Sindbis virus capsid-derived CPP (SVC4)
QAPKQPPKPKKPKTQEKKKKQPAKPKPGKRQRMALKLEADRLFDVKNEDGDVIGHALAMEGKVMKPLHVKGTIDHSS

SEQ ID NO: 17 - protein, 76 aa, Artificial Sequence
  Amino acid sequence of a Sindbis virus capsid-derived CPP (SVC5)
QATQEKKKKQPAKPKKPKTQEKKKKQPAKPKPGKRQRMALKLEADRLFDVKNEDGDVIGHALAMEGKVMKPLHVNG

SEQ ID NO: 18 - protein, 72 aa, Artificial Sequence
  Amino acid sequence of a Sindbis virus capsid-derived CPP (SVC6)
PKPQEKKKKQPAKPKKPKTQEKKKKQPAKPKPGKRQRMALKLEADRLFDVKNEDGDVICTQATRGQQRQIGR

SEQ ID NO: 19 - protein, 72 aa, Artificial Sequence
  Amino acid sequence of a Sindbis virus capsid-derived CPP (SVC7)
PKTQEKKKKQPAKPKPGKRQRMALKLEADRLFDVKNEDGDVIGHALAMEGKVMKPLHVKGTIDHPVLSKLIC

SEQ ID NO: 20 - protein, 70 aa, Artificial Sequence
  Amino acid sequence of a Sindbis virus capsid-derived CPP(SVC8)
HKKKAPKQPPKPKKPKTQEKKKKQPAKPKPGKRQRMALKLEADRLFDVKNEDGDVIGHALAMEGKVMKKQ

SEQ ID NO: 21 - protein, 69 aa, Artificial Sequence
  Amino acid sequence of a Sindbis virus capsid-derived CPP (SVC9)
LPPPRQKKQAPKQPPKPKKPKTQEKKKKQPAKPKPGKRQRMALKLEADRLSDVKNEDGDVIGHALARFV

SEQ ID NO: 22 - protein, 68 aa, Artificial Sequence
  Amino acid sequence of a Sindbis virus capsid-derived CPP (SVC10)
QKSQEKKKKQPAKPKKPKTQEKKKKQPAKPKPGKRQRMALKLEADRLFDVKNEDGDVIGHALAMEVKA

SEQ ID NO: 23 - protein, 67 aa, Artificial Sequence
  Amino acid sequence of a Sindbis virus capsid-derived CPP (SVC11)
PPRQKKQAPKQPPKPKKPKTQEKKRKQPAKPKPGKRQRMALKLEADRLFDVKNEDGDVIGHALARKA

SEQ ID NO: 24 - protein, 66 aa, Artificial Sequence
  Amino acid sequence of a Sindbis virus capsid-derived CPP (SVC12)
QTQEKKKKQPAKPKKPKTQEKKKKQPAKPKPGKRQRMALKLEADRLFDVKNEDGDVIGHALAMEAK

SEQ ID NO: 25 - protein, 66 aa, Artificial Sequence
  Amino acid sequence of a Sindbis virus capsid-derived CPP (SVC13)
QKKQPPKQPPKPKKPKTQEKKKKQPAKPKPGNRQRMALKLEADRLFDVKNEDGDVIGHALAMEGEA

SEQ ID NO: 26 - protein, 63 aa, Artificial Sequence
  Amino acid sequence of a Sindbis virus capsid-derived CPP (SVC14)
PKTQEKKKKQPAKPKPGKRQRMALKLEADRLFDVKNEDGDVIGQAPKQPPKPKKPKTQEKKKK

SEQ ID NO: 27 - protein, 61 aa, Artificial Sequence
  Amino acid sequence of a Sindbis virus capsid-derived CPP (SVC15)
QATKQPPKPKKPKTQEKKKKQPAKPKPGKRQRMALKLEADRLFDVKNEDGDVIGHALAMKT

SEQ ID NO: 28 - protein, 61 aa, Artificial Sequence
  Amino acid sequence of a Sindbis virus capsid-derived CPP (SVC16)
QAPKQPPKPKKPKTQEKKKKQPAKPKPGKRQRMALKLEADRLFDVKNEDGDVIGHALATKG

SEQ ID NO: 29 - protein, 61 aa, Artificial Sequence
  Amino acid sequence of a Sindbis virus capsid-derived CPP (SVC17)
LKTQEKKKKQPAKPKKPKTQEKKKKQAPKQPPKPKKPKTQEKKKKQPAKPKKPKTQEKKKA

SEQ ID NO: 30 - protein, 60 aa, Artificial Sequence
  Amino acid sequence of a Sindbis virus capsid-derived CPP (SVC18)
PKPKKPKTQEKKKKQPAKPKPGKRQRMALKLEADRLFDVKNEDGDVIGHALAMEGKVMKP

SEQ ID NO: 31 - protein, 59 aa, Artificial Sequence
  Amino acid sequence of a Sindbis virus capsid-derived CPP (SVC19)
PKQPPKPKKPKTQEKKKKQPAKPKPGKRQRMALKLEADRLFDVKNEDGDVIGHALDMKA

SEQ ID NO: 32 - protein, 52 aa, Artificial Sequence
  Amino acid sequence of a Sindbis virus capsid-derived CPP (SVC20)
PKQPPKPKKPKTQEKKKKQPAKPKKPKTQEKKKKQPAKPRPKTQEKKKKQPA

SEQ ID NO: 33 - protein, 52 aa, Artificial Sequence
  Amino acid sequence of a Sindbis virus capsid-derived CPP (SVC21)
QTAKQPPKPKKPKTQEKKKKQPAKPKPGKRQRMALKLEADRLFDVKNEDGGA

SEQ ID NO: 34 - protein, 52 aa, Artificial Sequence
  Amino acid sequence of a Sindbis virus capsid-derived CPP (SVC22)
PKTQEKKKKQAPKQPPKPKKPKTQEKKKKQPAKPKPGKRQRMALKLEADRVC

SEQ ID NO: 35 - protein, 51 aa, Artificial Sequence
  Amino acid sequence of a Sindbis virus capsid-derived CPP (SVC23)
QVKEKKQPAKPKKPKTQEKKKKQPAKPRPGKRQRMALKLEADRLFDVKDKG

SEQ ID NO: 36 - protein, 49 aa, Artificial Sequence
  Amino acid sequence of a Sindbis virus capsid-derived CPP (SVC24)
PKQPPKPKKPKTQEKKKKQPAKPKPGKRQRMALKLEADRLFDVKMKMAT

SEQ ID NO: 37 - protein, 46 aa, Artificial Sequence
  Amino acid sequence of a Sindbis virus capsid-derived CPP (SVC25)
PNPPEKKKKQPAKPKKPKTQEKKKKQPAKPKPGKRQRMALKLEEGA

SEQ ID NO: 38 - protein, 38 aa, Artificial Sequence
  Amino acid sequence of a Sindbis virus capsid-derived CPP (SVC26)
QAPKQPPKPKKPKTQEKKKKQPPKPKKPKTQEKKKKQA

SEQ ID NO: 39 - protein, 38 aa, Artificial Sequence
  Amino acid sequence of a Sindbis virus capsid-derived CPP (SVC27)
PLKPKKPKTQEKKKKQPPKPKKPKTQEKKKKQPPKPKR

SEQ ID NO: 40 - protein, 36 aa, Artificial Sequence
  Amino acid sequence of a Sindbis virus capsid-derived CPP (SVC28)
PNAQEKKKKQPPKPKKPKTQEKKKKQPPKPKKPKTQ

SEQ ID NO: 41 - protein, 36 aa, Artificial Sequence
  Amino acid sequence of a Sindbis virus capsid-derived CPP (SVC29)
PPKPKKPKTQEKKKKQPAKPKPGKRQRMALKLEAYA

SEQ ID NO: 42 - protein, 48 aa, Artificial Sequence
  Amino acid sequence of a CPP-Spy Tag fusion protein (CST1)
KTQEKKKKQPPKPKKPKTQEKKKKQPPKPKRGASAHIVMVDAYKPTKG

SEQ ID NO: 43 - protein, 33 aa, Artificial Sequence
  Amino acid sequence of a CPP-Spy Tag fusion protein (CST2)
PKKPKTQEKKKKQPPKGASAHIVMVDAYKPTKG

SEQ ID NO: 44 - protein, 38 aa, Artificial Sequence
  Amino acid sequence of a CPP-Spy Tag fusion protein (CST3)
PLKPKKPKTQEKKKKQPPKPKGASAHIVMVDAYKPTKG

SEQ ID NO: 45 - protein, 42 aa, Artificial Sequence
  Amino acid sequence of a CPP-Spy Tag fusion protein (CST4)
PLKPKKPKTQEKKKKQPPKPKKPKTGASAHIVMVDAYKPTKG

SEQ ID NO: 46 - protein, 44 aa, Artificial Sequence
  Amino acid sequence of a CPP-Spy Tag fusion protein (CST5)
PKTQEKKKKQPPKPKKPKTQEKKKKQPGASAHIVMVDAYKPTKG

SEQ ID NO: 47 - protein, 42 aa, Artificial Sequence
  Amino acid sequence of a CPP-Spy Tag fusion protein (CST6)
KKQPPKPKKPKTQEKKKKQPPKPKRGASAHIVMVDAYKPTKG

SEQ ID NO: 48 - protein, 44 aa, Artificial Sequence
  Amino acid sequence of a CPP-Spy Tag fusion protein (CST7)
PRQKKQAPKQPPKPKKPKTQEKKKKQPGASAHIVMVDAYKPTKG

SEQ ID NO: 49 - protein, 44 aa, Artificial Sequence
  Amino acid sequence of a CPP-Spy Tag fusion protein (CST8)
TKTQEKKKKQTTKTKKTKTQEKKKKQTGASAHIVMVDAYKPTKG

SEQ ID NO: 50 - protein, 44 aa, Artificial Sequence
  Amino acid sequence of a CPP-Spy Tag fusion protein (CST9)
PKTQEAAAAQPPKPKKPKTQEAAAAQPGASAHIVMVDAYKPTKG

SEQ ID NO: 51 - protein, 54 aa, Artificial Sequence
  Amino acid sequence of a CPP-Spy Tag fusion protein (CST10)
PLKPKKPKTQEKKKKQPPKPKKPKTQEKKKKQPPKPKGASAHIVMVDAYKPTKG

SEQ ID NO: 52 - protein, 53 aa, Artificial Sequence
  Amino acid sequence of a CPP-Spy Tag fusion protein (CST11)
KPKKPKTQEKKKKQPPKPKKPKTQEKKKKQPPKPKRGASAHIVMVDAYKPTKG

SEQ ID NO: 53 - protein, 51 aa, Artificial Sequence
  Amino acid sequence of a CPP-Spy Tag fusion protein (CST12)
KKPKTQEKKKKQPPKPKKPKTQEKKKKQPPKPKRGASAHIVMVDAYKPTKG

SEQ ID NO: 54 - protein, 49 aa, Artificial Sequence
  Amino acid sequence of a CPP-Spy Tag fusion protein (CST13)
KKQPPKPKKPKTQEKKKKQPPKPKKPKTQEKKGASAHIVMVDAYKPTKG

SEQ ID NO: 55 - protein, 55 aa, Artificial Sequence
  Amino acid sequence of a CPP-Spy Tag fusion protein (CST14)
TLKTKKTKTQEKKKKQTTKTKKTKTQEKKKKQTTKTKRGASAHIVMVDAYKPTKG

SEQ ID NO: 56 - protein, 55 aa, Artificial Sequence
  Amino acid sequence of a CPP-Spy Tag fusion protein (CST15)
PLRPRRPRTQERRRRQPPRPRRPRTQERRRRQPPRPRRGASAHIVMVDAYKPTKG

SEQ ID NO: 57 - protein, 48 aa, Artificial Sequence
  Amino acid sequence of a CPP-Spy Tag fusion protein (CST16)
KTQEKKKKQTTKTKKTKTQEKKKKQTTKTKRGASAHIVMVDAYKPTKG

SEQ ID NO: 58 - protein, 46 aa, Artificial Sequence
  Amino acid sequence of a CPP-Spy Tag fusion protein (CST17)
QEKKKKQPPKPKKPKTQEKKKKQPPKPKRGASAHIVMVDAYKPTKG

SEQ ID NO: 59 - protein, 44 aa, Artificial Sequence
  Amino acid sequence of a CPP-Spy Tag fusion protein (CST18)
KKKKQPPKPKKPKTQEKKKKQPPKPKRGASAHIVMVDAYKPTKG

SEQ ID NO: 60 - protein, 52 aa, Artificial Sequence
  Amino acid sequence of a CPP-Spy Tag fusion protein (CST19)
KSKKPKKKEKKHKEKERDKEKKKEKEKKKSPKPKKGASAHIVMVDAYKPTKG

SEQ ID NO: 61 - protein, 14 aa, Artificial Sequence
  Amino acid sequence of a proapoptotic peptide (PAP1)
KLAKLAKKLAKLAK

SEQ ID NO: 62 - protein, 132 aa, Artificial Sequence
  Amino acid sequence of a SpyCatcher-proapoptotic peptide fusion protein (SC-PAP)
MGHHHHHHGATLEVLFQGPGGSDSATHIKFSKRDEDGKELAGATMELRDSSGKTISTWISDGQVKDFYLYPGKYTFVETAAPDGYEVATAITFTVNEQGQVTVNGKATKGGSGTGATSGKLAKLAKKLAKLA

SEQ ID NO: 63 - protein, 18 aa, Artificial Sequence
  Amino acid sequence of a proapoptotic peptide (PAP2)
KLAKLAKKLAKLAKKLAK

SEQ ID NO: 64 - protein, 31 aa, Artificial Sequence
  Amino acid sequence of a Sindbis virus capsid-derived CPP (del1746)
KTQEKKKKQPPKPKKPKTQEKKKKQPPKPKR

SEQ ID NO: 65 - protein, 16 aa, Artificial Sequence
  Amino acid sequence of a Sindbis virus capsid-derived CPP (SAR1)
PKKPKTQEKKKKQPPK

SEQ ID NO: 66 - protein, 21 aa, Artificial Sequence
  Amino acid sequence of a Sindbis virus capsid-derived CPP (SAR2)
PLKPKKPKTQEKKKKQPPKPK

SEQ ID NO: 67 - protein, 25 aa, Artificial Sequence
  Amino acid sequence of a Sindbis virus capsid-derived CPP (SAR3)
PLKPKKPKTQEKKKKQPPKPKKPKT

SEQ ID NO: 68 - protein, 27 aa, Artificial Sequence
  Amino acid sequence of a Sindbis virus capsid-derived CPP (SAR4)
PKTQEKKKKQPPKPKKPKTQEKKKKQP

SEQ ID NO: 69 - protein, 25 aa, Artificial Sequence
  Amino acid sequence of a Sindbis virus capsid-derived CPP (SAR5)
KKQPPKPKKPKTQEKKKKQPPKPKR

SEQ ID NO: 70 - protein, 27 aa, Artificial Sequence
  Amino acid sequence of a Sindbis virus capsid-derived CPP (SAR6)
PRQKKQAPKQPPKPKKPKTQEKKKKQP

SEQ ID NO: 71 - protein, 27 aa, Artificial Sequence
  Amino acid sequence of a Sindbis virus capsid-derived CPP (SAR7)
TKTQEKKKKQTTKTKKTKTQEKKKKQT

SEQ ID NO: 72 - protein, 27 aa, Artificial Sequence
  Amino acid sequence of a Sindbis virus capsid-derived CPP (SAR9)
PKTQEAAAAQPPKPKKPKTQEAAAAQP

SEQ ID NO: 73 - protein, 37 aa, Artificial Sequence
  Amino acid sequence of a Sindbis virus capsid-derived CPP (SAR12)
PLKPKKPKTQEKKKKQPPKPKKPKTQEKKKKQPPKPK

SEQ ID NO: 74 - protein, 36 aa, Artificial Sequence
  Amino acid sequence of a Sindbis virus capsid-derived CPP (SAR13)
KPKKPKTQEKKKKQPPKPKKPKTQEKKKKQPPKPKR

SEQ ID NO: 75 - protein, 34 aa, Artificial Sequence
  Amino acid sequence of a Sindbis virus capsid-derived CPP (SAR14)
KKPKTQEKKKKQPPKPKKPKTQEKKKKQPPKPKR

SEQ ID NO: 76 - protein, 32 aa, Artificial Sequence
  Amino acid sequence of a Sindbis virus capsid-derived CPP (SAR15)
KKQPPKPKKPKTQEKKKKQPPKPKKPKTQEKK

SEQ ID NO: 77 - protein, 38 aa, Artificial Sequence
  Amino acid sequence of a Sindbis virus capsid-derived CPP (SAR16 P_T)
TLKTKKTKTQEKKKKQTTKTKKTKTQEKKKKQTTKTKR

SEQ ID NO: 78 - protein, 38 aa, Artificial Sequence
  Amino acid sequence of a Sindbis virus capsid-derived CPP (SAR 17)
PLRPRRPRTQERRRRQPPRPRRPRTQERRRRQPPRPRR

SEQ ID NO: 79 - protein, 31 aa, Artificial Sequence
  Amino acid sequence of a Sindbis virus capsid-derived CPP (1746P_T)
KTQEKKKKQTTKTKKTKTQEKKKKQTTKTKR

SEQ ID NO: 80 - protein, 29 aa, Artificial Sequence
  Amino acid sequence of a Sindbis virus capsid-derived CPP (1746-KT)
QEKKKKQPPKPKKPKTQEKKKKQPPKPKR

SEQ ID NO: 81 - protein, 27 aa, Artificial Sequence
  Amino acid sequence of a Sindbis virus capsid-derived CPP (1746.C27)
KKKKQPPKPKKPKTQEKKKKQPPKPKR

SEQ ID NO: 82 - protein, 35 aa, Artificial Sequence
  Amino acid sequence of a Sindbis virus capsid-derived CPP (SVC30)
KSKKPKKKEKKHKEKERDKEKKKEKEKKKSPKPKK

SEQ ID NO: 83 - protein, 119 aa, Artificial Sequence
  Amino acid sequence of SpyCatcher peptide (SCP)
MGHHHHHHGATLEVLFQGPGGSDSATHIKFSKRDEDGKELAGATMELRDSSGKTISTWISDGQVKDFYLYPGKYTFVETAAPDGYEVATAITFTVNEQGQVTVNGKATKGGSGTGATSG

SEQ ID NO: 84 - protein, 17 aa, Artificial Sequence
  Amino acid sequence of SpyTag peptide (STP)
GASAHIVMVDAYKPTKG

SEQ ID NO: 85 - protein, 51 aa, Artificial
  Amino acid sequence of a dominant negative ATF5 peptide
LEQRAEELARENEELLEKEAEELEQENAELEGECQGLEARNRELKERAESV

SEQ ID NO: 86 - protein, 15 aa, Artificial
  Amino acid sequence of a dominant negative ras-p21 peptide
YREQIKRVKDSDDVP

SEQ ID NO: 87 - protein, 18 aa, Artificial
  Amino acid sequence of a dominant negative ras-p21 peptide
TIEDSYRKQVVIDDNRAS

SEQ ID NO: 88 - protein, 43 aa, Artificial
  CPP represented by Formula I variable sequence
  VARIANT (1)..(1): none, Gln, Lys, or Arg
  VARIANT (2)..(2): none, Glu, or Thr
  VARIANT (3)..(3): none or Gln
  VARIANT (3)..(3): none or Gln
  VARIANT (4)..(4): none or Glu
  VARIANT (5)..(12): Lys or Arg
  VARIANT (5)..(12): Lys or Arg
  VARIANT (14)..(15): Pro, Thr, or Ala
  VARIANT (16)..(16): Lys or Arg
  VARIANT (17)..(17): Pro, Thr, Lys, or Gln
  VARIANT (18)..(19): Arg, Lys, or Pro
  VARIANT (20)..(20): Lys, Pro, or Thr
  VARIANT (21)..(21): Thr, Lys, Arg, or Pro
  VARIANT (22)..(22): Gln, Thr, or Lys
  VARIANT (23)..(23): Glu, Gln, or Lys
  VARIANT (24)..(24): Glu or Pro
  VARIANT (29)..(36): Lys or Arg
  VARIANT (38)..(39): Thr or Pro
  VARIANT (40)..(40): Lys or Arg
  VARIANT (41)..(41): Pro or Thr
  VARIANT (42)..(42): Lys or Arg
XXXXXXXXXXXXQXXXXXXXXXXXKTQEXXXXXXXXQXXXXXR

SEQ ID NO: 89 - protein, 25 aa, Artificial Sequence
  Amino acid sequence of a CPP variant
PKTQEKKKKQPPKPKKPKTQEKKKK

SEQ ID NO: 90 - protein, 392 aa, Artificial Sequence
  Amino acid sequence of SpyCatcher-Beta lactamase fusion protein
MGHHHHHHGATLEVLFQGPGGSGSDSATHIKFSKRDEDGKELAGATMELRDSSGKTISTWISDGQVKDFYLYPGKYTFVETAAPDGYEVATAITFTVNEQGQVTVNGKATKGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWQLGSASGTTGATSGEF

SEQ ID NO: 91 - protein, 419 aa, ARTIFICIAL SEQUENCE
  Amino acid sequence of SpyC BLA-FPP1.1 fusion protein
MGHHHHHHGATLEVLFQGPGGSGSDSATHIKFSKRDEDGKELAGATMELRDSSGKTISTWISDGQVKDFYLYPGKYTFVETAAPDGYEVATAITFTVNEQGQVTVNGKATKGSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWQLGSASGTTGATSGEFKKKKQPPKPKKPKTQEKKKKQPPKPKR

SEQ ID NO: 92 - protein, 16 aa, ARTIFICIAL SEQUENCE
  Amino acid sequence of PenetratinCPP
RQIKIWFQNRRMKWKK

SEQ ID NO: 93 - protein, 10 aa, ARTIFICIAL SEQUENCE
  Amino acid sequence of TAT CPP
GRKKRRQRRR

SEQ ID NO: 94 - protein, 23 aa, ARTIFICIAL SEQUENCE
  Amino acid sequence of LL1-SpyTag fusion
GGTAGSTGGAHIVMVDAYKPTKG

SEQ ID NO: 95 - protein, 27 aa, ARTIFICIAL SEQUENCE
  Amino acid sequence TAT-Spytag fusion
GRKKRRQRRRGASAHIVMVDAYKPTKG

SEQ ID NO: 96 - protein, 33 aa, ARTIFICIAL SEQUENCE
  Amino acid sequence of TAT-SpyTag fusion
RQIKIWFQNRRMKWKKGASAHIVMVDAYKPTKG

SEQ ID NO: 97 - protein, 44 aa, ARTIFICIAL SEQUENCE; identical to SEQ ID NO: 59
  Amino acid sequence of FPP1.1-SpyTag

SEQ ID NO: 98 - protein, 125 aa, ARTIFICIAL SEQUENCE
  Amino acid sequence of FPP1.1-Omomyc fusion
KKKKQPPKPKKPKTQEKKKKQPPKPKRHHHHHHSDTEENVKRRTHNVLERQRRNELKRSFFALRDQIPELENNEKAPKVVILKKATAYILSVQAETQKLISEIDLLRKQNEQLKHKLEQLRNSCA

SEQ ID NO: 99 - protein, 102 aa, ARTIFICIAL SEQUENCE
  Amino acid sequence of Omomyc
GPGGSGTGATSDTEENVKRRTHNVLERQRRNELKRSFFALRDQIPELENNEKAPKVVILKKATAYILSVQAETQKLISEIDLLRKQNEQLKHKLEQLRNSCA

SEQ ID NO: 100 - protein, 161 aa, ARTIFICIAL SEQUENCE
  Amino acid sequence of FPP1.1 SpyCycle fusion
MASKKKKQPPKPKKPKTQEKKKKQPPKPKRASSGSHHHHHHGATLEVLFQGPGGSDSATHIKFSKRDEDGKELAGATMELRDSSGKTISTWISDGQVKDFYLYPGKYTFVETAAPDGYEVATAITFTVNEQGQVTVNGKATKGTSGAGKPIPNPLLGLDST

SEQ ID NO: 101 - protein, 73 aa, ARTIFICIAL SEQUENCE
  Amino acid sequence of FPP1.1-dimer SpyTag fusion
KKKKQPPKPKKPKTQEKKKKQPPKPKRGASAHIVMVDAYKPTKGASKKKKQPPKPKKPKTQEKKKKQPPKPKR

SEQ ID NO: 102 - protein, 55 aa, ARTIFICIAL SEQUENCE
  Amino acid sequence of FPP1-SpyTag fusion
PLKPKKPKTQEKKKKQPPKPKKPKTQEKKKKQPPKPKRGASAHIVMVDAYKPTKG

SEQ ID NO: 103 - protein, 50 aa, ARTIFICIAL SEQUENCE
  Amino acid sequence  of FPP2-SpyTag fusion
RKQKSLQTKLAENPPVPRKKRQSRPRWKQWLQKGASAHIVMVDAYKPTKG

SEQ ID NO: 104 - protein, 64 aa, ARTIFICIAL SEQUENCE
  Amino acid sequence of FPP3- SpyTag fusion protein
PPHPRPLPAPAQSRKKQKGRAGRGHEKTGASVLRGPQKPHPLPAQLRGASAHIVMVDAYKPTKG

SEQ ID NO: 105 - protein, 12 aa, ARTIFICIAL SEQUENCE
  Amino acid sequence of PMII peptide (D-Amino acid peptide)
  MISC_FEATURE (1)..(12): Amino Acids are D-form amino acids
TNWYANLEKLLR

SEQ ID NO: 106 - protein, 53 aa, ARTIFICIAL SEQUENCE
  Amino acid sequence of FPP1_DPMII peptide (FPP1_DPMI-I)
  MISC (41)..(53): Residues from 41-53 are D-form amino acids
PLKPKKPKTQEKKKKQPPKPKKPKTQEKKKKQPPKPKRGASTNWYANLEKLLR

SEQ ID NO: 107 - protein, 25 aa, ARTIFICIAL SEQUENCE
  TAT_D-PMII peptide
GRKKRRQRRRGASTNWYANLEKLLR

SEQ ID NO: 108 - protein, 48 aa, ARTIFICIAL SEQUENCE
  FPP3_DPMII peptide fusion
  MISC (37)..(48): Residues 37 to 48 are D-form amino acids
RKQKSLQTKLAENPPVPRKKRQSRPRWKQWLQKGASTNWYANLEKLLR

SEQ ID NO: 109 - protein, 477 aa, ARTIFICIAL SEQUENCE
  Amino acid sequence of EGFRAffBd-Boug-SpyCycle fusion
MGHHHHHHGATLEVLFQGPGGSGSVDNKFNKEMWAAWEEIRNLPNLNGWQMTAFIASLVDDPSQSANLLAEAKKLNDAQAPKGTGSGTGSATSGSLAGSGATAGTGSGYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKGSGATAGSAATGGATGGSDSATHIKFSKRDEDGKELAGATMELRDSSGKTISTWISDGQVKDFYLYPGKYTFVETAAPDGYEVATAITFTVNEQGQVTVNGKATKGGAGSWSHPQFEKG

SEQ ID NO: 110 - protein, 230 aa, ARTIFICIAL SEQUENCE
  Amino acid sequence of SpyC_Omomyc fusion
MASHHHHHHGATLEVLFQGPGGSDSATHIKFSKRDEDGKELAGATMELRDSSGKTISTWISDGQVKDFYLYPGKYTFVETAAPDGYEVATAITFTVNEQGQVTVNGKATKGGSGTGATSGSDTEENVKRRTHNVLERQRRNELKRSFFALRDQIPELENNEKAPKVVILKKATAYILSVQAETQKLISEIDLLRKQNEQLKHKLEQLRNSCATSGAGKPIPNPLLGLDST

SEQ ID NO: 111 - protein, 78 aa, ARTIFICIAL SEQUENCE
  Amino acid sequence of FPP1.1_Avitag_SpyTag fusion
KKKKQPPKPKKPKTQEKKKKQPPKPKRGASGLNDIFEAQKIEWHEGASAHIVMVDAYKPTKGASGKPIPNPLLGLDST

SEQ ID NO: 112 - protein, 263 aa, ARTIFICIAL SEQUENCE
  Amino acid sequence of  -lactamase (BLA)
HPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 113 - protein, 28 aa, ARTIFICIAL SEQUENCE
  Amino acid sequence of SAR20  (SAR20)
PKKKKQPPKPKKPKTQEKKKKQPPKPKR

SEQ ID NO: 114 - protein, 27 aa, ARTIFICIAL SEQUENCE
  Amino acid sequence of SAR21  (SAR21)
QPPKPKRKKKKQPPKPKKPKTQEKKKK

SEQ ID NO: 115 - protein, 27 aa, ARTIFICIAL SEQUENCE
  Amino acid sequence of SAR22  (SAR22)
RKPKPPQKKKKEQTKPKKPKPPQKKKK

SEQ ID NO: 116 - protein, 27 aa, ARTIFICIAL SEQUENCE
  Amino acid sequence of SAR23  (SAR23)
KKKKQPPKPKKPKTQEKKKKQPPKPKR

SEQ ID NO: 117 - protein, 27 aa, ARTIFICIAL SEQUENCE
  Amino acid sequence of SAR24
  MISC (1)..(4): Residues 1-4 are D-form amino acids
KKKKQPPKPKKPKTQEKKKKQPPKPKR

SEQ ID NO: 118 - protein, 27 aa, ARTIFICIAL SEQUENCE
  Amino acid sequence of SAR25
  MISC (1)..(24): Residues 1, 3, 8, 11, 17, 19, and 24 are D-form amino acids
KKKKQPPKPKKPKTQEKKKKQPPKPKR

SEQ ID NO: 119 - protein, 27 aa, ARTIFICIAL SEQUENCE
  Amino acid sequence of SAR26
KRRKQPPKPKKPKTQEKRRKQPPKPKR

SEQ ID NO: 120 - protein, 27 aa, ARTIFICIAL SEQUENCE
  Amino acid sequence of SAR27
RKKRQPPKPKKPKTQERKKRQPPKPKR

SEQ ID NO: 121 - protein, 27 aa, ARTIFICIAL SEQUENCE
  Amino acid sequence of SAR28
KPPKQPPKPKKPKTQEKPPKQPPKPKR

SEQ ID NO: 122 - protein, 27 aa, ARTIFICIAL SEQUENCE
  Amino acid sequence of SAR29
PKKPQPPKPKKPKTQEPKKPQPPKPKR

SEQ ID NO: 123 - protein, 27 aa, ARTIFICIAL SEQUENCE
  Amino acid sequence of SAR30
KKKKEPPKPKKPKTEEKKKKEPPKPKR

SEQ ID NO: 124 - protein, 28 aa, ARTIFICIAL SEQUENCE
  Amino acid sequence of SAR31
PEEEEQPPKPKKPKTQEEEEEQPPKPKR

SEQ ID NO: 125 - protein, 27 aa, ARTIFICIAL SEQUENCE
  Amino acid sequence of SAR32
KKKKQPPEPEEPETQEKKKKQPPEPER

SEQ ID NO: 126 - protein, 27 aa, ARTIFICIAL SEQUENCE
  Amino acid sequence of SAR33
KKKKQPPAPAAPATQEKKKKQPPAPAR

SEQ ID NO: 127 - protein, 27 aa, ARTIFICIAL SEQUENCE
  Amino acid sequence of SAR34
KKKKQPPQPQQPQTQEKKKKQPPQPQR

SEQ ID NO: 128 - protein, 27 aa, ARTIFICIAL SEQUENCE
  Amino acid sequence of SAR35
KKKKQPPTPTTPTTQEKKKKQPPTPTR

SEQ ID NO: 129 - protein, 27 aa, ARTIFICIAL SEQUENCE
  Amino acid sequence of SAR36
KKKKQTTKTKKTKTQEKKKKQTTKTKR

SEQ ID NO: 130 - protein, 27 aa, ARTIFICIAL SEQUENCE
  Amino acid sequence of SAR37
KKKKQAAKAKKAKTQEKKKKQAAKAKR

SEQ ID NO: 131 - protein, 39 aa, ARTIFICIAL SEQUENCE
  Amino acid sequence of SAR 38  (SAR38)
KKKKQPPKPKKPKTQEQPPKPKKPKTQEKKKKQPPKPKR

SEQ ID NO: 132 - protein, 39 aa, ARTIFICIAL SEQUENCE
  Amino acid sequence of SAR39
KKKKQTTKTKKTKTQEQTTKTKKTKTQEKKKKQTTKTKR

SEQ ID NO: 133 - protein, 39 aa, ARTIFICIAL SEQUENCE
  Amino acid sequence of SAR40
KKKKQPPKPKKPKTKKKKQPPKPKKPKTKKKKQPPKPKR

SEQ ID NO: 134 - protein, 27 aa, ARTIFICIAL SEQUENCE
  Amino acid sequence of SAR41
KKKKQPPKPKKKKTQEKKKKQPPKPKR

SEQ ID NO: 135 - protein, 27 aa, ARTIFICIAL SEQUENCE
  Amino acid sequence of SAR42
KKKKQPPKKKKPKTQEKKKKQPPKPKR

SEQ ID NO: 136 - protein, 27 aa, ARTIFICIAL SEQUENCE
  Amino acid sequence of SAR43
KKKKQPPKKKKKKTQEKKKKQPPKPKR

SEQ ID NO: 137 - protein, 27 aa, ARTIFICIAL SEQUENCE
  Amino acid sequence of SAR44
KKKKQPPKPKKPKTQEKKKKQPPKKKR

SEQ ID NO: 138 - protein, 25 aa, ARTIFICIAL SEQUENCE
  Amino acid sequence of SAR45
KKKQPPKPKKPKTQEKKKQPPKPKR

SEQ ID NO: 139 - protein, 29 aa, ARTIFICIAL SEQUENCE
  Amino acid sequence of SAR46
KKKKKQPPKPKKPKTQEKKKKKQPPKPKR

SEQ ID NO: 140 - protein, 31 aa, ARTIFICIAL SEQUENCE
  Amino acid sequence of SAR47
KKKKKKQPPKPKKPKTQEKKKKKKQPPKPKR

SEQ ID NO: 141 - protein, 33 aa, ARTIFICIAL SEQUENCE
  Amino acid sequence of SAR48
KKKKKKKQPPKPKKPKTQEKKKKKKKQPPKPKR

SEQ ID NO: 142 - protein, 35 aa, ARTIFICIAL SEQUENCE
  Amino acid sequence of SAR49
KKKKKKKKQPPKPKKPKTQEKKKKKKKKQPPKPKR

SEQ ID NO: 143 - protein, 29 aa, ARTIFICIAL SEQUENCE
  Amino acid sequence of SAR50
KKKKKKQPPKPKKPKTQEKKKKQPPKPKR

SEQ ID NO: 144 - protein, 29 aa, ARTIFICIAL SEQUENCE
  Amino acid sequence of SAR51
KKKKQPPKPKKPKTQEKKKKKKQPPKPKR

SEQ ID NO: 145 - protein, 31 aa, ARTIFICIAL SEQUENCE
  Amino acid sequence of SAR52
KKKKKKKKQPPKPKKPKTQEKKKKQPPKPKR

SEQ ID NO: 146 - protein, 31 aa, ARTIFICIAL SEQUENCE
  Amino acid sequence of SAR53
KKKKQPPKPKKPKTQEKKKKKKKKQPPKPKR

SEQ ID NO: 147 - protein, 27 aa, ARTIFICIAL SEQUENCE
  Amino acid sequence of SAR54
KKKKKPPKPKKPKTQEKKKKQPPKPKR

SEQ ID NO: 148 - protein, 27 aa, ARTIFICIAL SEQUENCE
  Amino acid sequence of SAR55
KKKKKKPKPKKPKTQEKKKKQPPKPKR

SEQ ID NO: 149 - protein, 28 aa, ARTIFICIAL SEQUENCE
  Amino acid sequence of SAR56
KKKKKKKPKPKKPKTQEKKKKQPPKPKR

SEQ ID NO: 150 - protein, 27 aa, ARTIFICIAL SEQUENCE
  Amino acid sequence of SAR57
KKKKKKKKPKKPKTQEKKKKQPPKPKR

SEQ ID NO: 151 - protein, 27 aa, ARTIFICIAL SEQUENCE
  Amino acid sequence of SAR58
KKKKKKKKPKKPKTQEKKKKKPPKPKR

SEQ ID NO: 152 - protein, 27 aa, ARTIFICIAL SEQUENCE
  Amino acid sequence of SAR59
KKKKKKKKPKKPKTQEKKKKKKPKPKR

SEQ ID NO: 153 - protein, 27 aa, ARTIFICIAL SEQUENCE
  Amino acid sequence of SAR60
KKKKKKKKPKKPKTQKKKKKKKPKPKR

SEQ ID NO: 154 - protein, 27 aa, ARTIFICIAL SEQUENCE
  Amino acid sequence of SAR61
KKKKKKKKPKKPKTQEKKKKKKKKPKR

SEQ ID NO: 155 - protein, 28 aa, ARTIFICIAL SEQUENCE
  Amino acid sequence of SAR62
KKKKKKKPKPKKPKTKKKKKKKKPKPKR

SEQ ID NO: 156 - protein, 27 aa, ARTIFICIAL SEQUENCE
  Amino acid sequence of SAR63
KKKKKKPKPKKPKTKKKKKKKKPKPKR

SEQ ID NO: 157 - protein, 27 aa, ARTIFICIAL SEQUENCE
  Amino acid sequence of SAR64
KKKKQPPKPKKPKTKKKKKKKKPKPKR

SEQ ID NO: 158 - protein, 27 aa, ARTIFICIAL SEQUENCE
  Amino acid sequence of SAR65
KKKKQPPKPKKPKTKKKKKKKKPKPKR

SEQ ID NO: 159 - protein, 27 aa, ARTIFICIAL SEQUENCE
  Amino acid sequence of SAR66
  MOD_RES (1)..(4): Orn
  MOD_RES (17)..(20): Orn
XXXXQPPKPKKPKTQEXXXXQPPKPKR

SEQ ID NO: 160 - protein, 27 aa, ARTIFICIAL SEQUENCE
  Amino acid sequence of SAR67
  MOD_RES (1)..(4): Orn
  MOD_RES (8)..(8): Orn
  MOD_RES (10)..(11): Orn
  MOD_RES (13)..(13): Orn
  MOD_RES (17)..(20): Orn
  MOD_RES (24)..(24): Orn
  MOD_RES (26)..(26): Orn
XXXXQPPXPXXPXTQEXXXXQPPXPXR

SEQ ID NO: 161 - protein, 27 aa, ARTIFICIAL SEQUENCE
  Amino acid sequence of SAR68
  MOD_RES (1)..(4): citrulline
  MOD_RES (17)..(20): citrulline
XXXXQPPKPKKPKTQEXXXXQPPKPKR

SEQ ID NO: 162 - protein, 27 aa, ARTIFICIAL SEQUENCE
  Amino acid sequence of SAR69
  MOD_RES (1)..(4): citrulline
  MOD_RES (8)..(8): citrulline
  MOD_RES (10)..(11): citrulline
  MOD_RES (13)..(13): citrulline
  MOD_RES (17)..(20): citrulline
  MOD_RES (24)..(24): citrulline
  MOD_RES (26)..(26): citrulline
XXXXQPPXPXXPXTQEXXXXQPPXPXR

SEQ ID NO: 163 - protein, 27 aa, ARTIFICIAL SEQUENCE
  Amino acid sequence of SAR70
  MOD_RES (1)..(4): Dbu
  MOD_RES (17)..(20): Dbu
XXXXQPPKPKKPKTQEXXXXQPPKPKR

SEQ ID NO: 164 - protein, 27 aa, ARTIFICIAL SEQUENCE
  Amino acid sequence of SAR71
  MOD_RES (1)..(4): Dbu
  MOD_RES (8)..(8): Dbu
  MOD_RES (10)..(11): Dbu
  MOD_RES (13)..(13): Dbu
  MOD_RES (17)..(20): Dbu
  MOD_RES (24)..(24): Dbu
  MOD_RES (26)..(26): Dbu
XXXXQPPXPXXPXTQEXXXXQPPXPXR

SEQ ID NO: 165 - protein, 27 aa, ARTIFICIAL SEQUENCE
  Amino acid sequence of SAR72
  MOD_RES (1)..(4): Dpr
  MOD_RES (17)..(20): Dpr
XXXXQPPKPKKPKTQEXXXXQPPKPKR

SEQ ID NO: 166 - protein, 27 aa, ARTIFICIAL SEQUENCE
  Amino acid sequence of SAR73
  MOD_RES (1)..(4): Dpr
  MOD_RES (8)..(8): Dpr
  MOD_RES (10)..(11): Dpr
  MOD_RES (13)..(13): Dpr
  MOD_RES (17)..(20): Dpr
  MOD_RES (24)..(24): Dpr
  MOD_RES (26)..(26): Dpr
XXXXQPPXPXXPXTQEXXXXQPPXPXR

SEQ ID NO: 167 - protein, 27 aa, ARTIFICIAL SEQUENCE
  Amino acid sequence of SAR74
  MOD_RES (6)..(7): D-proline
  MOD_RES (9)..(9): D-proline
  MOD_RES (12)..(12): D-proline
  MOD_RES (22)..(23): D-proline
  MOD_RES (25)..(25): D-proline
KKKKQXXKXKKXKTQEKKKKQXXKXKR

SEQ ID NO: 168 - protein, 45 aa, ARTIFICIAL SEQUENCE
  Amino acid sequence of SAR20-SpyTag fusion protein
PKKKKQPPKPKKPKTQEKKKKQPPKPKRGASAHIVMVDAYKPTKG

SEQ ID NO: 169 - protein, 44 aa, ARTIFICIAL SEQUENCE
  Amino acid sequence of SAR21-SpyTag fusion protein
QPPKPKRKKKKQPPKPKKPKTQEKKKKGASAHIVMVDAYKPTKG

SEQ ID NO: 170 - protein, 44 aa, ARTIFICIAL SEQUENCE
  Amino acid sequence of SAR22-SpyTag fusion protein
RKPKPPQKKKKEQTKPKKPKPPQKKKKGASAHIVMVDAYKPTKG

SEQ ID NO: 171 - protein, 44 aa, ARTIFICIAL SEQUENCE
  Amino acid sequence of SAR23-SpyTag fusion protein
  MOD_RES (1)..(4): D-Lysine
  MOD_RES (17)..(20): D-Lysine
XXXXQPPKPKKPKTQEXXXXQPPKPKRGASAHIVMVDAYKPTKG

SEQ ID NO: 172 - protein, 44 aa, ARTIFICIAL SEQUENCE
  Amino acid sequence of SAR24-SpyTag fusion protein
  MOD_RES (1)..(4): D-Lysine
XXXXQPPKPKKPKTQEKKKKQPPKPKRGASAHIVMVDAYKPTKG

SEQ ID NO: 173 - protein, 44 aa, ARTIFICIAL SEQUENCE
  Amino acid sequence of SAR25-SpyTag fusion protein
  MOD_RES (1)..(1): D-Lysine
  MOD_RES (3)..(3): D-Lysine
  MOD_RES (8)..(8): D-Lysine
  MOD_RES (11)..(11): D-Lysine
  MOD_RES (17)..(17): D-Lysine
  MOD_RES (19)..(19): D-Lysine
  MOD_RES (24)..(24): D-Lysine
XKXKQPPXPKXPKTQEXKXKQPPXPKRGASAHIVMVDAYKPTKG

SEQ ID NO: 174 - protein, 44 aa, ARTIFICIAL SEQUENCE
  Amino acid sequence of SAR26-SpyTag fusion protein
KRRKQPPKPKKPKTQEKRRKQPPKPKRGASAHIVMVDAYKPTKG

SEQ ID NO: 175 - protein, 44 aa, ARTIFICIAL SEQUENCE
  Amino acid sequence of SAR27-SpyTag fusion protein
RKKRQPPKPKKPKTQERKKRQPPKPKRGASAHIVMVDAYKPTKG

SEQ ID NO: 176 - protein, 44 aa, ARTIFICIAL SEQUENCE
  Amino acid sequence of SAR28-SpyTag fusion protein
KPPKQPPKPKKPKTQEKPPKQPPKPKRGASAHIVMVDAYKPTKG

SEQ ID NO: 177 - protein, 44 aa, ARTIFICIAL SEQUENCE
  Amino acid sequence of SAR29-SpyTag fusion protein
PKKPQPPKPKKPKTQEPKKPQPPKPKRGASAHIVMVDAYKPTKG

SEQ ID NO: 178 - protein, 44 aa, ARTIFICIAL SEQUENCE
  Amino acid sequence of SAR30-SpyTag fusion protein
KKKKEPPKPKKPKTEEKKKKEPPKPKRGASAHIVMVDAYKPTKG

SEQ ID NO: 179 - protein, 45 aa, ARTIFICIAL SEQUENCE
  Amino acid sequence of SAR31-SpyTag fusion protein
PEEEEQPPKPKKPKTQEEEEEQPPKPKRGASAHIVMVDAYKPTKG

SEQ ID NO: 180 - protein, 44 aa, ARTIFICIAL SEQUENCE
  Amino acid sequence of SAR32-SpyTag fusion protein
KKKKQPPEPEEPETQEKKKKQPPEPERGASAHIVMVDAYKPTKG

SEQ ID NO: 181 - protein, 44 aa, ARTIFICIAL SEQUENCE
  Amino acid sequence of SAR33-SpyTag fusion protein
KKKKQPPAPAAPATQEKKKKQPPAPARGASAHIVMVDAYKPTKG

SEQ ID NO: 182 - protein, 44 aa, ARTIFICIAL SEQUENCE
  Amino acid sequence of SAR34-SpyTag fusion protein
KKKKQPPQPQQPQTQEKKKKQPPQPQRGASAHIVMVDAYKPTKG

SEQ ID NO: 183 - protein, 44 aa, ARTIFICIAL SEQUENCE
  Amino acid sequence of SAR35-SpyTag fusion protein
KKKKQPPTPTTPTTQEKKKKQPPTPTRGASAHIVMVDAYKPTKG

SEQ ID NO: 184 - protein, 44 aa, ARTIFICIAL SEQUENCE
  Amino acid sequence of SAR36-SpyTag fusion protein
KKKKQTTKTKKTKTQEKKKKQTTKTKRGASAHIVMVDAYKPTKG

SEQ ID NO: 185 - protein, 44 aa, ARTIFICIAL SEQUENCE
  Amino acid sequence of SAR37-SpyTag fusion protein
KKKKQAAKAKKAKTQEKKKKQAAKAKRGASAHIVMVDAYKPTKG

SEQ ID NO: 186 - protein, 56 aa, ARTIFICIAL SEQUENCE
  Amino acid sequence of SAR38-SpyTag fusion protein
KKKKQPPKPKKPKTQEQPPKPKKPKTQEKKKKQPPKPKRGASAHIVMVDAYKPTKG

SEQ ID NO: 187 - protein, 56 aa, ARTIFICIAL SEQUENCE
  Amino acid sequence of SAR39-SpyTag fusion protein
KKKKQTTKTKKTKTQEQTTKTKKTKTQEKKKKQTTKTKRGASAHIVMVDAYKPTKG

SEQ ID NO: 188 - protein, 56 aa, ARTIFICIAL SEQUENCE
  Amino acid sequence of SAR40-SpyTag fusion protein
KKKKQPPKPKKPKTKKKKQPPKPKKPKTKKKKQPPKPKRGASAHIVMVDAYKPTKG

SEQ ID NO: 189 - protein, 44 aa, ARTIFICIAL SEQUENCE
  Amino acid sequence of SAR41-SpyTag fusion protein
KKKKQPPKPKKKKTQEKKKKQPPKPKRGASAHIVMVDAYKPTKG

SEQ ID NO: 190 - protein, 44 aa, ARTIFICIAL SEQUENCE
  Amino acid sequence of SAR42-SpyTag fusion protein
KKKKQPPKKKKPKTQEKKKKQPPKPKRGASAHIVMVDAYKPTKG

SEQ ID NO: 191 - protein, 44 aa, ARTIFICIAL SEQUENCE
  Amino acid sequence of SAR43-SpyTag fusion protein
KKKKQPPKKKKKKTQEKKKKQPPKPKRGASAHIVMVDAYKPTKG

SEQ ID NO: 192 - protein, 44 aa, ARTIFICIAL SEQUENCE
  Amino acid sequence of SAR44-SpyTag fusion protein
KKKKQPPKPKKPKTQEKKKKQPPKKKRGASAHIVMVDAYKPTKG

SEQ ID NO: 193 - protein, 42 aa, ARTIFICIAL SEQUENCE
  Amino acid sequence of SAR45-SpyTag fusion protein
KKKQPPKPKKPKTQEKKKQPPKPKRGASAHIVMVDAYKPTKG

SEQ ID NO: 194 - protein, 46 aa, ARTIFICIAL SEQUENCE
  Amino acid sequence of SAR46-SpyTag fusion protein
KKKKKQPPKPKKPKTQEKKKKKQPPKPKRGASAHIVMVDAYKPTKG

SEQ ID NO: 195 - protein, 48 aa, ARTIFICIAL SEQUENCE
  Amino acid sequence of SAR47-SpyTag fusion protein
KKKKKKQPPKPKKPKTQEKKKKKKQPPKPKRGASAHIVMVDAYKPTKG

SEQ ID NO: 196 - protein, 50 aa, ARTIFICIAL SEQUENCE
  Amino acid sequence of SAR48-SpyTag fusion protein
KKKKKKKQPPKPKKPKTQEKKKKKKKQPPKPKRGASAHIVMVDAYKPTKG

SEQ ID NO: 197 - protein, 52 aa, ARTIFICIAL SEQUENCE
  Amino acid sequence of SAR49-SpyTag fusion protein
KKKKKKKKQPPKPKKPKTQEKKKKKKKKQPPKPKRGASAHIVMVDAYKPTKG

SEQ ID NO: 198 - protein, 46 aa, ARTIFICIAL SEQUENCE
  Amino acid sequence of SAR50-SpyTag fusion protein
KKKKKKQPPKPKKPKTQEKKKKQPPKPKRGASAHIVMVDAYKPTKG

SEQ ID NO: 199 - protein, 46 aa, ARTIFICIAL SEQUENCE
  Amino acid sequence of SAR51-SpyTag fusion protein
KKKKQPPKPKKPKTQEKKKKKKQPPKPKRGASAHIVMVDAYKPTKG

SEQ ID NO: 200 - protein, 48 aa, ARTIFICIAL SEQUENCE
  Amino acid sequence of SAR52-SpyTag fusion protein
KKKKKKKKQPPKPKKPKTQEKKKKQPPKPKRGASAHIVMVDAYKPTKG

SEQ ID NO: 201 - protein, 48 aa, ARTIFICIAL SEQUENCE
  Amino acid sequence of SAR53-SpyTag fusion protein
KKKKQPPKPKKPKTQEKKKKKKKKQPPKPKRGASAHIVMVDAYKPTKG

SEQ ID NO: 202 - protein, 44 aa, ARTIFICIAL SEQUENCE
  Amino acid sequence of SAR54-SpyTag fusion protein
KKKKKPPKPKKPKTQEKKKKQPPKPKRGASAHIVMVDAYKPTKG

SEQ ID NO: 203 - protein, 44 aa, ARTIFICIAL SEQUENCE
  Amino acid sequence of SAR55-SpyTag fusion protein
KKKKKKPKPKKPKTQEKKKKQPPKPKRGASAHIVMVDAYKPTKG

SEQ ID NO: 204 - protein, 45 aa, ARTIFICIAL SEQUENCE
  Amino acid sequence of SAR56-SpyTag fusion protein
KKKKKKKPKPKKPKTQEKKKKQPPKPKRGASAHIVMVDAYKPTKG

SEQ ID NO: 205 - protein, 44 aa, ARTIFICIAL SEQUENCE
  Amino acid sequence of SAR57-SpyTag fusion protein
KKKKKKKKPKKPKTQEKKKKQPPKPKRGASAHIVMVDAYKPTKG

SEQ ID NO: 206 - protein, 44 aa, ARTIFICIAL SEQUENCE
  Amino acid sequence of SAR58-SpyTag fusion protein
KKKKKKKKPKKPKTQEKKKKKPPKPKRGASAHIVMVDAYKPTKG

SEQ ID NO: 207 - protein, 44 aa, ARTIFICIAL SEQUENCE
  Amino acid sequence of SAR59-SpyTag fusion protein
KKKKKKKKPKKPKTQEKKKKKKPKPKRGASAHIVMVDAYKPTKG

SEQ ID NO: 208 - protein, 44 aa, ARTIFICIAL SEQUENCE
  Amino acid sequence of SAR60-SpyTag fusion protein
KKKKKKKKPKKPKTQKKKKKKKPKPKRGASAHIVMVDAYKPTKG

SEQ ID NO: 209 - protein, 44 aa, ARTIFICIAL SEQUENCE
  Amino acid sequence of SAR61-SpyTag fusion protein
KKKKKKKKPKKPKTQEKKKKKKKKPKRGASAHIVMVDAYKPTKG

SEQ ID NO: 210 - protein, 45 aa, ARTIFICIAL SEQUENCE
  Amino acid sequence of SAR62-SpyTag fusion protein
KKKKKKKPKPKKPKTKKKKKKKKPKPKRGASAHIVMVDAYKPTKG

SEQ ID NO: 211 - protein, 44 aa, ARTIFICIAL SEQUENCE
  Amino acid sequence of SAR63-SpyTag fusion protein
KKKKKKPKPKKPKTKKKKKKKKPKPKRGASAHIVMVDAYKPTKG

SEQ ID NO: 212 - protein, 44 aa, ARTIFICIAL SEQUENCE
  Amino acid sequence of SAR64-SpyTag fusion protein
KKKKKPPKPKKPKTKKKKKKKKPKPKRGASAHIVMVDAYKPTKG

SEQ ID NO: 213 - protein, 44 aa, ARTIFICIAL SEQUENCE
  Amino acid sequence of SAR65-SpyTag fusion protein
KKKKQPPKPKKPKTKKKKKKKKPKPKRGASAHIVMVDAYKPTKG

SEQ ID NO: 214 - protein, 44 aa, ARTIFICIAL SEQUENCE; identical to SEQ ID NO: 171
  Amino acid sequence of SAR66-SpyTag fusion protein
  MOD_RES (1)..(4): Orn
  MOD_RES (17)..(20): Orn

SEQ ID NO: 215 - protein, 43 aa, ARTIFICIAL SEQUENCE
  Amino acid sequence of SAR67-SpyTag fusion protein
  MOD_RES (1)..(4): Orn
  MOD_RES (8)..(8): Orn
  MOD_RES (10)..(11): Orn
  MOD_RES (13)..(13): Orn
  MOD_RES (17)..(20): Orn
  MOD_RES (24)..(24): Orn
  MOD_RES (26)..(26): Orn
XXXXQPPXPXXPXTQEXXXXQPPXPXRGASAHIVMVDAYPTKG

SEQ ID NO: 216 - protein, 44 aa, ARTIFICIAL SEQUENCE; identical to SEQ ID NO: 171
  Amino acid sequence of SAR68-SpyTag fusion protein
  MOD_RES (1)..(4): X = citrulline
  MOD_RES (17)..(20): X = citrulline

SEQ ID NO: 217 - protein, 44 aa, ARTIFICIAL SEQUENCE
  Amino acid sequence of SAR69-SpyTag fusion protein  (SAR69-ST)
  MOD_RES (1)..(4): X = citrulline
  MOD_RES (8)..(8): X = citrulline
  MOD_RES (10)..(11): X = citrulline
  MOD_RES (13)..(13): X = citrulline
  MOD_RES (17)..(20): X = citrulline
  MOD_RES (24)..(24): X = citrulline
  MOD_RES (26)..(26): X = citrulline
XXXXQPPXPXXPXTQEXXXXQPPXPXRGASAHIVMVDAYKPTKG

SEQ ID NO: 218 - protein, 43 aa, ARTIFICIAL SEQUENCE
  Amino acid sequence of SAR70-SpyTag fusion protein
  MOD_RES (1)..(4): Dbu
  MOD_RES (17)..(20): Dbu
XXXXQPPKPKKPKTQEXXXXQPPKPKRGASHIVMVDAYKPTKG

SEQ ID NO: 219 - protein, 44 aa, ARTIFICIAL SEQUENCE; identical to SEQ ID NO: 217
  Amino acid sequence of SAR71-SpyTag fusion protein
  MOD_RES (1)..(4): Dbu
  MOD_RES (8)..(8): Dbu
  MOD_RES (10)..(11): Dbu
  MOD_RES (13)..(13): Dbu
  MOD_RES (17)..(20): Dbu
  MOD_RES (24)..(24): Dbu
  MOD_RES (26)..(26): Dbu

SEQ ID NO: 220 - protein, 44 aa, ARTIFICIAL SEQUENCE; identical to SEQ ID NO: 171
  Amino acid sequence of SAR72-SpyTag fusion protein
  MOD_RES (1)..(4): Dpr
  MOD_RES (17)..(20): Dpr

SEQ ID NO: 221 - protein, 44 aa, ARTIFICIAL SEQUENCE; identical to SEQ ID NO: 217
  Amino acid sequence of SAR73-SpyTag fusion protein
  MOD_RES (1)..(4): Dpr
  MOD_RES (8)..(8): Dpr
  MOD_RES (10)..(11): Dpr
  MOD_RES (13)..(13): Dpr
  MOD_RES (17)..(20): Dpr
  MOD_RES (24)..(24): Dpr
  MOD_RES (26)..(26): Dpr

SEQ ID NO: 222 - protein, 44 aa, ARTIFICIAL SEQUENCE
  Amino acid sequence of SAR74-SpyTag fusion protein
  MOD_RES (6)..(7): X = D-form proline
  MOD_RES (9)..(9): X = D-form proline
  MOD_RES (12)..(12): X = D-form proline
  MOD_RES (22)..(23): X = D-form proline
  MOD_RES (25)..(25): X = D-form proline
KKKKQXXKXKKXKTQEKKKKQXXKXKRGASAHIVMVDAYKPTKG

SEQ ID NO: 223 - protein, 7 aa, ARTIFICIAL SEQUENCE
  CPP partial amino acid sequence of Formula I (X1-C)
QPPKPKR

SEQ ID NO: 224 - protein, 7 aa, ARTIFICIAL SEQUENCE
  CPP partial amino acid sequence of Formula I (X1-D)
RKPKPPQ

SEQ ID NO: 225 - protein, 12 aa, ARTIFICIAL SEQUENCE
  CPP partial amino acid sequence of Formula I (X3-G)
  MOD_RES (4)..(4): X = D-form lysine
  MOD_RES (7)..(7): X = D-form lysine
QPPXPKXPKTQE

SEQ ID NO: 226 - protein, 12 aa, ARTIFICIAL SEQUENCE
  CPP partial amino acid sequence of Formula I (X3-H)
EPPKPKKPKTEE

SEQ ID NO: 227 - protein, 12 aa, ARTIFICIAL SEQUENCE
  CPP partial amino acid sequence of Formula I (X3-I)
QPPEPEEPETQE

SEQ ID NO: 228 - protein, 12 aa, ARTIFICIAL SEQUENCE
  CPP partial amino acid sequence of Formula I (X3-J)
QPPAPAAPATQE

SEQ ID NO: 229 - protein, 12 aa, ARTIFICIAL SEQUENCE
  CPP partial amino acid sequence of Formula I (X3-K)
QPPQPQQPQTQE

SEQ ID NO: 230 - protein, 12 aa, ARTIFICIAL SEQUENCE
  CPP partial amino acid sequence of Formula I (X3-L)
QPPTPTTPTTQE

SEQ ID NO: 231 - protein, 12 aa, ARTIFICIAL SEQUENCE
  CPP partial amino acid sequence of Formula I (X3-M)
QAAKAKKAKTQE

SEQ ID NO: 232 - protein, 24 aa, ARTIFICIAL SEQUENCE
  CPP partial amino acid sequence of Formula I (X3-N)
QPPKPKKPKTQEQPPKPKKPKTQE

SEQ ID NO: 233 - protein, 24 aa, ARTIFICIAL SEQUENCE
  CPP partial amino acid sequence of Formula I (X3-O)
QTTKTKKTKTQEQTTKTKKTKTQE

SEQ ID NO: 234 - protein, 24 aa, ARTIFICIAL SEQUENCE
  CPP partial amino acid sequence of Formula I (X3-P)
QPPKPKKPKTKKKKQPPKPKKPKT

SEQ ID NO: 235 - protein, 12 aa, ARTIFICIAL SEQUENCE
  CPP partial amino acid sequence of Formula I (X3-Q)
QPPKPKKKKTQE

SEQ ID NO: 236 - protein, 12 aa, ARTIFICIAL SEQUENCE
  CPP partial amino acid sequence of Formula I (X3-R)
QPPKKKKPKTQE

SEQ ID NO: 237 - protein, 12 aa, ARTIFICIAL SEQUENCE
  CPP partial amino acid sequence of Formula I (X3-S)
QPPKKKKKKTQE

SEQ ID NO: 238 - protein, 11 aa, ARTIFICIAL SEQUENCE
  CPP partial amino acid sequence of Formula I (X3-T)
PPKPKKPKTQE

SEQ ID NO: 239 - protein, 10 aa, ARTIFICIAL SEQUENCE
  CPP partial amino acid sequence of Formula I (X3-U)
PKPKKPKTQE

SEQ ID NO: 240 - protein, 8 aa, ARTIFICIAL SEQUENCE
  CPP partial amino acid sequence of Formula I (X3-V)
PKKPKTQE

SEQ ID NO: 241 - protein, 7 aa, ARTIFICIAL SEQUENCE
  CPP partial amino acid sequence of Formula I (X3-W)
PKKPKTQ

SEQ ID NO: 242 - protein, 8 aa, ARTIFICIAL SEQUENCE
  CPP partial amino acid sequence of Formula I (X3-X)
PKPKKPKT

SEQ ID NO: 243 - protein, 9 aa, ARTIFICIAL SEQUENCE
  CPP partial amino acid sequence of Formula I (X3-Y)
PPKPKKPKT

SEQ ID NO: 244 - protein, 12 aa, ARTIFICIAL SEQUENCE
  CPP partial amino acid sequence of Formula I (X3-Z)
  MOD_RES (4)..(4): Orn
  MOD_RES (6)..(7): Orn
  MOD_RES (9)..(9): Orn
QPPXPXXPXTQE

SEQ ID NO: 245 - protein, 12 aa, ARTIFICIAL SEQUENCE
  CPP partial amino acid sequence of Formula I (X3-AA)
  MOD_RES (4)..(4): X = citrulline
  MOD_RES (6)..(7): X = citrulline
  MOD_RES (9)..(9): X = citrulline
QPPXPXXPXTQE

SEQ ID NO: 246 - protein, 12 aa, ARTIFICIAL SEQUENCE
  CPP partial amino acid sequence of Formula I (X3-AB)
  MOD_RES (4)..(4): Dbu
  MOD_RES (6)..(7): Dbu
  MOD_RES (9)..(9): Dbu
QPPXPXXPXTQE

SEQ ID NO: 247 - protein, 12 aa, ARTIFICIAL SEQUENCE
  CPP partial amino acid sequence of Formula I (X3-AC)
  MOD_RES (4)..(4): Dpr
  MOD_RES (6)..(7): Dpr
  MOD_RES (9)..(9): Dpr
QPPXPXXPXTQE

SEQ ID NO: 248 - protein, 12 aa, ARTIFICIAL SEQUENCE
  CPP partial amino acid sequence of Formula I (X3-AD)
  MOD_RES (2)..(3): X = D-form proline
  MOD_RES (5)..(5): X = D-form proline
  MOD_RES (8)..(8): X = D-form proline
QXXKXKKXKTQE

SEQ ID NO: 249 - protein, 7 aa, ARTIFICIAL SEQUENCE
  CPP partial amino acid sequence of Formula I (X5-E)
  misc_feature (4)..(4): Xaa can be any naturally occurring amino acid
QPPXPKR

SEQ ID NO: 250 - protein, 7 aa, ARTIFICIAL SEQUENCE
  CPP partial amino acid sequence of Formula I (X5-F)
QPPEPER

SEQ ID NO: 251 - protein, 7 aa, ARTIFICIAL SEQUENCE
  CPP partial amino acid sequence of Formula I (X5-G)
QPPAPAR

SEQ ID NO: 252 - protein, 7 aa, ARTIFICIAL SEQUENCE`
  CPP partial amino acid sequence of Formula I (X5-H)
QPPQPQR

SEQ ID NO: 253 - protein, 7 aa, ARTIFICIAL SEQUENCE
  CPP partial amino acid sequence of Formula I (X5-I)
QPPTPTR

SEQ ID NO: 254 - protein, 7 aa, ARTIFICIAL SEQUENCE
  CPP partial amino acid sequence of Formula I (X5-J)
QTTKTKR

SEQ ID NO: 255 - protein, 7 aa, ARTIFICIAL SEQUENCE
  CPP partial amino acid sequence of Formula I (X5-K)
QAAKAKR

SEQ ID NO: 256 - protein, 7 aa, ARTIFICIAL SEQUENCE
  CPP partial amino acid sequence of Formula I (X5-L)
QPPKKKR

SEQ ID NO: 257 - protein, 6 aa, ARTIFICIAL SEQUENCE
  CPP partial amino acid sequence of Formula I (X5-M)
PPKPKR

SEQ ID NO: 258 - protein, 5 aa, ARTIFICIAL SEQUENCE
  CPP partial amino acid sequence of Formula I (X5-N)
PKPKR

SEQ ID NO: 259 - protein, 7 aa, ARTIFICIAL SEQUENCE
  CPP partial amino acid sequence of Formula I (X5-P)
  MOD_RES (4)..(4): Orn
  MOD_RES (6)..(6): Orn
QPPXPXR

SEQ ID NO: 260 - protein, 7 aa, ARTIFICIAL SEQUENCE
  CPP partial amino acid sequence of Formula I (X5-Q)
  MOD_RES (4)..(4): X = citrulline
  MOD_RES (6)..(6): X = citrulline
QPPXPXR

SEQ ID NO: 261 - protein, 5 aa, ARTIFICIAL SEQUENCE
  Amino acid sequence of a flexible linker (LKR1)
GGGGS

SEQ ID NO: 262 - protein, 10 aa, ARTIFICIAL SEQUENCE
  Amino acid sequence of a flexible linker (LKR2)
GGGGSGGGGS

SEQ ID NO: 263 - protein, 9 aa, ARTIFICIAL SEQUENCE
  Amino acid sequence of a flexible linker (LKR3)
GGTAGSTGG